(19) United States
(12) Patent Application Publication
(10) Pub. No.: US 2026/0365739 A1
(43) Pub. Date: Nov. 19, 2026

(54) IMPROVED ENGINEERED IMMUNOGLOBULINS AND BINDING FRAGMENTS

(71) Applicant: National Research Council of Canada, Ottawa (CA)

(72) Inventors: Dae Young KIM, Orleans (CA); Jamshid TANHA, Orleans (CA)

(73) Assignee: National Research Council of Canada, Ottawa, ON (CA)

(21) Appl. No.: 19/125,491

(22) PCT Filed: Oct. 30, 2023
(86) PCT No.: PCT/IB2023/060921

§ 371 Date: Apr. 29, 2025

(51) Int. Cl. C07K 16/28; C07K 16/1282; C12N 15/10

(52) U.S. Cl. CPC C07K 16/2863; C07K 16/1282; C12N 15/1093; C07K 2317/565; C07K 2317/567; C07K 2317/569; C07K 2317/624; C07K 2317/92; C07K 2317/94

(57) ABSTRACT

Provided herein is an engineered immunoglobulin (Ig) variable domain comprising or capable of comprising at least one non-canonical disulfide bond formed between a first cysteine and a second cysteine, wherein the first and second cysteines are selected from a cysteine residue introduced at position 24, position 86, position 22, position 88, position 4, position 25, position 118, position 5, position 120, position 6, position 119, position 45, and/or position 100, wherein the position numbering refers to IMGT numbering, and wherein the first and second cysteines are not at the same position. Methods of making and using said engineered Ig variable domain(s) are also provided.

### CROSS-REFERENCE TO RELATED APPLICATIONS

This application claims the benefit of U.S. provisional application No. 63/381,624, filed Oct. 31, 2022, the contents of which is herein incorporated by reference in its entirety.

### REFERENCE TO AN ELECTRONIC SEQUENCE LISTING

The contents of the electronic sequence listing (2019-087-02_SL_270ct2023.xml; Size: 573,615 bytes; and Date of Creation: Oct. 27, 2023) is herein incorporated by reference in its entirety.

### FIELD

The present disclosure relates to engineered immunoglobulins (Igs) and binding fragments thereof, and in particular to engineered Igs and antigen-binding fragments thereof that have improved stability comprising one or more non-canonical disulfide bond(s).

### BACKGROUND

Intra- and inter-domain disulfide linkages play critical roles in the folding and stability of antibodies and other large, secreted macromolecules (1). Depending on isotype, human immunoglobulin (Ig) molecules contain between 14 and 23 disulfide linkages, including a highly conserved intradomain disulfide linkage connecting β-strands B and F at the core of each Ig domain (2). Incorporation of exogenous disulfide linkages into proteins can enhance their thermodynamic and kinetic stability (3), which, for therapeutic antibodies, is associated with improved manufacturability, safety, and efficacy (4).

Incorporation of engineered disulfide linkages spanning β-strands A and G (Cys4-Cys119 and Cys6-Cys119; IMGT numbering used throughout (45)) improved the thermostability of the Ig constant (C) C_{H}2 domain (5). In Ig variable (V) domains, which form the antigen-combining sites of antibodies, the canonical B-F strand intradomain disulfide linkage is formed between Cys23-Cys104. Rarely, Ig V domains contain additional non-canonical disulfide linkages formed between Cys pairs located in framework regions (FRs): naturally occurring non-canonical linkages have been identified spanning Cys54-Cys78 (β-strands C′-D; 6,7) and Cys40-Cys55 (β-strands C-C′; 8,9), and a non-canonical linkage was successfully engineered between Cys39-Cys87 (β-strands C-E) based on structural modeling (7). The Ig V domains of some vertebrates (e.g., camelids; chickens; cows; sharks) contain non-canonical disulfide linkages at much higher frequencies (10), but these almost exclusively involve at least one Cys located in a complementarity-determining region (CDR). Incorporation of one or more non-canonical disulfide linkage(s) into the FRs of single or multiple Ig domains can additively improve the thermostability of both the individual domains and the whole IgG molecules containing them (11,12). Similar stabilizing effects have been observed for non-canonical disulfide linkages formed between positions within or structurally analogous to the CDR loops (13, 14), but these cannot be tolerated in all Ig V domains without compromising antigen binding.

### SUMMARY

Most immunoglobulin (Ig) domains bear only a single highly conserved canonical intradomain disulfide linkage formed between Cys23-Cys104, and incorporation of rare non-canonical disulfide linkages at other locations can enhance Ig domain stability. Here, the sequence tolerance of Ig variable (V) domain framework regions (FRs) to non-canonical disulfide linkages is exhaustively surveyed. This approach identified seven novel Cys pairs in V_{H} FRs (Cys24-Cys86, Cys22-Cys88, Cys4-Cys25, Cys4-Cys118, Cys5-Cys120, Cys6-Cys119 and Cys45-Cys100 by IMGT numbering) whose presence putatively resulted in formation of non-canonical disulfide linkages (four intra- and three inter-β sheet), rescuing domain folding and stability. None of the non-canonical disulfide linkages were present in the natural human V_{H} repertoire. These data reveal an unexpected permissiveness of Ig V domains to non-canonical disulfide linkages at diverse locations in FRs, absent in the human repertoire, whose presence is compatible with antigen recognition and improves domain biophysical properties.

Accordingly, an aspect of the disclosure includes an engineered immunoglobulin (Ig) variable domain comprising an introduced pair of cysteine residues comprising a first cysteine residue and a second cysteine residue, wherein:
the first cysteine residue is at position 24 and the second cysteine residue is at position 86;
the first cysteine residue is at position 22 and the second cysteine residue is at position 88;
the first cysteine residue is at position 4 and the second cysteine residue is at position 25;
the first cysteine residue is at position 4 and the second cysteine residue is at position 118;
the first cysteine residue is at position 5 and the second cysteine residue is at position 120;
the first cysteine residue is at position 6 and the second cysteine residue is at position 119; or
the first cysteine residue is at position 45 and the second cysteine residue is at position 100,
   wherein the position numbering refers to IMGT numbering and wherein at least one disulfide bond is formed or is capable of forming between the first and second cysteine residues.

In an embodiment, the variable domain is a heavy chain variable domain. In another embodiment, the variable domain is a V_{H}H variable domain, a light chain variable domain, or a V_{NAR} variable domain.

In an embodiment, the engineered Ig variable domain comprises any one of the amino acid sequences set forth in SEQ ID NOs: 332-338, 340-346, 348-354, 356-362, 364-370, 372-378, 380-386, 388-394, 396-402, 404-410, 412-418, 420-426, 428-434, 436-442, 444-450, 452-458, 460-466, 468-474, 476-482, 484-490, 492-498, 500-506, 508-514, 516-522, 524-530, 532-538, 540-546, 548-554, 556-562, 564-570, 572-578, 580-586, 588-594, and 596-602 or an amino acid sequence having at least 80% sequence identity to any one of the amino acid sequences set forth in SEQ ID NOs: 332-338, 340-346, 348-354, 356-362, 364-370, 372-378, 380-386, 388-394, 396-402, 404-410, 412-418, 420-426, 428-434, 436-442, 444-450, 452-458, 460-466, 468-474, 476-482, 484-490, 492-498, 500-506, 508-514, 516-522, 524-530, 532-538, 540-546, 548-554, 556-562, 564-570, 572-578, 580-586, 588-594, and 596-602, wherein the first and second cysteine residues are maintained.

In an embodiment, the pair of cysteine residues is introduced by mutating a nucleic acid molecule encoding the engineered Ig variable domain.

Another aspect of the disclosure includes an antibody or antigen-binding fragment thereof comprising at least one engineered Ig variable domain as described herein.

In an embodiment, the antibody or antigen-binding fragment thereof is a single domain antibody, optionally a nanobody. In another embodiment, the antibody or antigen-binding fragment thereof is an IgG1, IgG2, IgG3, IgG4, IgE, IgA, IgY, IgD, IgM, and/or IgNAR antibody. In an embodiment, the antibody or antigen-binding fragment thereof is an antibody fragment selected from Fab, Fab′, F(ab′)2, scFv, dsFv, ds-scFv, dimers, minibodies, diabodies, and multimers thereof.

Another aspect of the disclosure includes a polypeptide comprising a plurality of engineered Ig variable domains as described herein.

Another aspect of the disclosure includes a nucleic acid molecule encoding an engineered Ig variable domain as described herein, an antibody or antigen-binding fragment thereof as described herein, or a polypeptide as described herein. The nucleic acid molecule may be comprised in a vector. The nucleic acid molecule may, for example, be incorporated into an expression cassette or expression vector for expression of the antibody binding fragment. Accordingly, an aspect of the disclosure includes an expression cassette or a vector, optionally an expression vector, comprising a nucleic acid molecule as described herein. Suitable expression vectors include but are not limited to cosmids, plasmids, or modified viruses (e.g., replication defective retroviruses, adenoviruses and adeno-associated viruses).

Another aspect of the disclosure includes a vector comprising a nucleic acid molecule as described herein.

Another aspect of the disclosure includes a cell comprising a nucleic acid molecule as described herein. In an embodiment, the cell is a mammalian cell. In a further embodiment, the cell is a human embryonic kidney (HEK) cell or a Chinese hamster ovary (CHO) cell.

Another aspect of the disclosure includes an animal comprising a nucleic acid molecule as described herein. In an embodiment, the animal is a non-human mammal or bird. In an embodiment, the animal is a transgenic or genetically engineered animal, including a progeny or descendant thereof that comprises the nucleic acid molecule.

Another aspect of the disclosure includes a cell expressing an engineered Ig variable domain as described herein, an antibody or antigen-binding fragment thereof as described herein, a polypeptide as described herein, a nucleic acid molecule as described herein, a vector as described herein, or an expression cassette as described herein.

Another aspect of the disclosure includes an animal expressing an engineered Ig variable domain as described herein, an antibody or antigen-binding fragment thereof as described herein, a polypeptide as described herein, a nucleic acid molecule as described herein, a vector as described herein, or an expression cassette as described herein. In an embodiment, the animal is a non-human mammal or bird. In an embodiment, the animal is a transgenic or genetically engineered animal, including a progeny or descendant thereof that comprises and expresses the engineered Ig variable domain, the antibody or binding fragment, the polypeptide, the nucleic acid molecule, the vector, or the expression cassette.

Another aspect of the disclosure includes a composition comprising an engineered Ig variable domain as described herein, an antibody or antigen-binding fragment thereof as described herein, a polypeptide as described herein, a nucleic acid molecule as described herein, a vector as described herein, or an expression cassette as described herein, and optionally comprising a pharmaceutically acceptable carrier and/or excipient.

Another aspect of the disclosure includes a kit comprising an engineered Ig variable domain as described herein, an antibody or antigen-binding fragment thereof as described herein, a polypeptide as described herein, a nucleic acid molecule as described herein, a vector as described herein, or an expression cassette as described herein and packaging material.

Another aspect of the disclosure includes an engineered library comprising at least one nucleic acid molecule encoding at least one engineered Ig variable domain as described herein or encoding at least one antibody or antigen-binding fragment thereof as described herein. In some embodiments, the at least one engineered Ig variable domain or the at least one antibody or antigen-binding fragment thereof is a plurality and wherein at least a subset of the plurality comprises different CDR sequences. In some embodiments, each nucleic acid molecule in the library encodes an engineered Ig variable domain as described herein. In other embodiments, only a subset of nucleic acid molecules in the library encode an engineered Ig variable domain as described herein, while other nucleic acid molecules in the library encode different Ig variable domains. In some embodiments, the library is a display library; such as a phage display library, a ribosome display library, a yeast display library, a mammalian cell display library, or a bacterial display library. In an embodiment, the library comprises a plurality of nucleic acid molecules, each encoding an engineered Ig variable domain or an antibody or antigen-binding fragment thereof, wherein the Ig variable domains or the antibodies or antigen-binding fragments thereof encoded by the nucleic acid molecules comprise the same engineered cysteine residues but have different framework and/or CDR sequences.

Another aspect of disclosure includes a method of making an antibody or antigen-binding fragment thereof having increased stability relative to a control, the method comprising generating an antibody or antigen-binding fragment thereof comprising at least one of the engineered Ig variable domains as described herein, wherein the control is an antibody or antigen-binding fragment thereof into which the pair of cysteine residues has not been introduced but is otherwise the same as the antibody or antigen-binding fragment thereof produced by the method.

Another aspect of the disclosure includes a method of increasing stability of an antibody or antigen-binding fragment thereof relative to a control, the method comprising introducing into at least one variable domain of the antibody or antigen-binding fragment thereof a pair of cysteine residues, wherein the pair of cysteine residues is selected from:
a cysteine residue at position 24 and a cysteine residue at position 86;
a cysteine residue at position 22 and a cysteine residue at position 88;
a cysteine residue at position 4 and a cysteine residue at position 25;
a cysteine residue at position 4 and a cysteine residue at position 118;
a cysteine residue at position 5 and a cysteine residue at position 120;
a cysteine residue at position 6 and a cysteine residue at position 119; and
a cysteine residue at position 45 and a cysteine residue at position 100, wherein:
   the position numbering refers to IMGT numbering,
   the cysteine residues introduced form or are capable of forming a disulfide bond, and
   the control is an antibody or antigen-binding fragment thereof into which the pair of cysteine residues has not been introduced but is otherwise the same as the antibody or antigen-binding fragment thereof produced by the method.

In some embodiments, the variable domain is a heavy chain variable domain. In other embodiments, the variable domain is a V_{H}H variable domain, a light chain variable domain, or a V_{NAR} variable domain.

In particular embodiments, the engineered Ig variable domain comprises any one of the amino acid sequences of SEQ ID NOs: 332-338, 340-346, 348-354, 356-362, 364-370, 372-378, 380-386, 388-394, 396-402, 404-410, 412-418, 420-426, 428-434, 436-442, 444-450, 452-458, 460-466, 468-474, 476-482, 484-490, 492-498, 500-506, 508-514, 516-522, 524-530, 532-538, 540-546, 548-554, 556-562, 564-570, 572-578, 580-586, 588-594, and 596-602 or an amino acid sequence having at least 80%, at least 85% at least 90% or at least 95% sequence identity to any one of the amino acid sequences of SEQ ID NOs: 332-338, 340-346, 348-354, 356-362, 364-370, 372-378, 380-386, 388-394, 396-402, 404-410, 412-418, 420-426, 428-434, 436-442, 444-450, 452-458, 460-466, 468-474, 476-482, 484-490, 492-498, 500-506, 508-514, 516-522, 524-530, 532-538, 540-546, 548-554, 556-562, 564-570, 572-578, 580-586, 588-594, and 596-602, wherein the one or more than one cysteine pair is maintained.

In some embodiments, the resulting antibody or antigen-binding fragment thereof has increased thermostability and/or increased resistance to protease activity relative to the control.

Another aspect of the disclosure includes a method for identifying one or more than one location in an engineered immunoglobulin (Ig) variable domain tolerant of disulfide bonds, the method comprising
a) generating a library of nucleic acid molecules encoding an engineered immunoglobulin (Ig) variable domain lacking a cysteine residue at position 23 and/or lacking a cysteine residue at position 104 and thus lacking a disulfide bond between position 23 and position 104, wherein at least one cysteine residue has been introduced into the amino acid sequence of each engineered Ig variable domain encoded by the nucleic acid molecules of the library;
b) identifying Ig variable domains encoded by the nucleic acid molecules of the library which have formed one or more than one disulfide bond other than between positions 23 and 104; and
c) identifying the positions between which the one or more than one disulfide bond is formed in each engineered Ig variable domain, wherein the position numbering refers to IMGT numbering.

Another aspect of the disclosure includes a method of identifying engineered immunoglobulin (Ig) variable domain disulfide bonds that increase stability or solubility of the Ig variable domain, the method comprising:
a) generating a library of nucleic acid molecules encoding engineered immunoglobulin (Ig) variable domains lacking a cysteine residue at position 23 and/or lacking a cysteine residue at position 104 and thus lacking a disulfide bond between position 23 and position 104, wherein engineered pairs of cysteine residues have been introduced into the amino acid sequence of each engineered Ig variable domain encoded by the nucleic acid molecules of the library;
b) identifying Ig variable domains encoded by the nucleic acid molecules of the library which have formed at least one disulfide bond other than between positions 23 and 104;
c) determining the stability of the identified Ig variable domains, and
d) identifying the positions between which the disulfide bonds are formed in each engineered Ig variable domain that has increased stability as compared to a control,
   wherein the position numbering refers to IMGT numbering. In an embodiment of the method, the stability is determined by measuring thermostability and/or protease resistance of the engineered Ig variable domain.

In some embodiments, generating a library of nucleic acid molecules comprises generating a library of nucleic acid molecules encoding an engineered Ig variable domain lacking a cysteine residue at position 23 and/or lacking a cysteine residue at position 104. In some embodiments, the library is generated using Kunkel mutagenesis.

In some embodiments, the Ig variable domains, encoded by the nucleic acid molecules of the library, that have formed at least one disulfide bond other than between positions 23 and 104, are identified using phage display.

In some embodiments, identifying the positions between which the disulfide bonds are formed comprises next generation sequencing.

In some embodiments, the variable domain is a heavy chain variable domain.

In some embodiments, the variable domain is a V_{H}H variable domain, a light chain variable domain, or a V_{NAR} variable domain.

### BRIEF DESCRIPTION OF DRAWINGS

An embodiment of the present disclosure will now be described in relation to the drawings in which:

FIGS. 1A-1D. FIG. 1A provides a cartoon representation showing rescue of a destabilized Cys23-Cys104 null immunoglobulin V_{H} domain by Cys pair scanning of FRs and enrichment of phage-displayed V_{H}S bearing non-canonical disulfide linkages from libraries using protein A selection. FIG. 1B shows panning of fd phage displaying V_{H}S or their Cys23-Cys104 null derivatives against protein A. Only VH413^{C23-C104 null} phage showed a significant difference in post-selection output phage titer compared with the WT V_{H}-displaying phage (arrow heads). Pre, pre-selection; Post, post-selection. FIG. 1C shows the effect of introducing known canonical (Cys23-Cys104) and non-canonical (Cys40-Cys55 and Cys54-Cys78) disulfide linkages into VH413^{C23-C104 null} fd phage on output phage titer following protein A selection. “None” refers to unmodified VH413^{C23-C104 null} displaying fd phage (black bar and dotted line). The effects of a negative control Cys pair (Cys54-Cys87) were also assessed. FIG. 1D shows panning of test VH413^{C23-C104 null} Cys pair scan libraries (B-F, C-C′ and C′-D) against protein A. The percent frequencies of known non-canonical stabilizing disulfide linkages formed between these β-strands is indicated. Four rounds of panning were performed. The pannings in FIGS. 1B, 1C and 1D were performed at room temperature with no heating step. ‘Library’ denotes the unpanned libraries. cfu, colony-forming units.

FIGS. 2A and 2B. FIG. 2A shows the effect of introducing of eight Cys pairs forming putative non-canonical FR disulfide linkages on the Tₘs of two V_{H}s (VH413 and VH428) and two V_{H}Hs (A4.2 and A26.8). Tₘ was measured using a differential scanning fluorimetry. Four negative control Cys pairs predicted not to form disulfide linkages were also assessed. FIG. 2B shows the effect of introducing seven Cys pairs forming putative non-canonical FR disulfide linkages on Ig V domain thermostability, other properties and antigen recognition. V_{H}Hs are shown in open squares, V_{H}S in open circles, and V_{L}s in X symbols. Expression (%) refers to the proportion of sdAbs expressing with adequate yields for subsequent experiments. Changes in sdAb Tₘ and fraction refolded (α-value) following introduction of FR Cys pairs were measured by circular dichroism. Changes in sdAb monomericity following introduction of FR Cys pairs were measured by SEC-MALS. Changes in the K_{D}s of antigen specific V_{H}Hs following introduction of FR Cys pairs were assessed by SPR. For changes (Δ) in biophysical properties, values above the null (Δ_{Monomer} 0%, ΔTₘ 0 °C, and Δα-value 0) indicate improvement, while for changes in binding affinity, values above the null (ΔK_{D} 1) reflect weaker binding. Median values are indicated by black lines.

FIG. 3 shows the effect of introducing Cys pairs forming putative non-canonical FR disulfide linkages on V_{H}H pepsin resistance. The three Cys pairs conferring the highest increase in sdAb Tₘ (Cys24-Cys86, Cys4-Cys25 and Cys6-Cys119) were assessed as well as one Cys pair (Cys22-Cys88) that had an intermediate effect on Tₘ. The V_{H}Hs were digested with three concentrations of pepsin for 1 h at 37 °C, and then the remaining undigested V_{H}H was quantitated by SDS-PAGE and densitometry. Lines indicate averages for each Cys pair.

FIGS. 4A-4E show the presence of non-canonical Cys residues in the human expressed V_{H} repertoires of 17 individuals. FIG. 4A shows frequency of non-canonical Cys residues in V_{H} FRs and CDRs, disregarding IMGT positions 23 and 104. FIG. 4B shows frequency of expression V_{H}s bearing the indicated number of Cys residues. This analysis includes Cys23/Cys104, and thus the majority of V_{H}S bear two Cys residues. FIG. 4C provides a circos plot showing the relative frequencies and strands of non-canonical Cys pairs in the subset of expressed human V_{H}s bearing ≥4 FR Cys residues. The size of each β-strand along the circle's circumference reflects the frequency of non-canonical Cys, and the overlap shows the relative frequency of the second Cys in the pair. Data represent median values across 17 V_{H} repertoires. FIG. 4D shows most frequent β-strands bearing non-canonical Cys pairs among human V_{H}s bearing ≥4 FR Cys residues. FIG. 4E shows most frequent non-canonical Cys pairs among human V_{H}s bearing ≥4 FR Cys residues. Lines represent medians. Note that y axes are log-scaled and zero values are not plotted.

FIG. 5 shows LC-MS analysis of free sulfhydryl abundance in WT and Cys-engineered V_{H}Hs. For each category of V_{H}H (WT or the indicated engineered Cys pair), the percentage of reduced and non-reduced protein analyzed bearing 0, 2, and 4 maleimide-PEG2-biotin labels is indicated (left y-axis; sum: 100%). The molar ratio of free sulhydryl groups to total V_{H}H protein is shown in black dotted line (right y-axis). Error bars represent standard errors of the means. Abbreviations: G, guanidine-HCl; L, label (maleimide-PEG2-biotin); T, TCEP.

### DETAILED DESCRIPTION

### I. Definitions

As used herein, the following terms may have meanings ascribed to them below, unless specified otherwise. However, it should be understood that other meanings that are known or understood by those having ordinary skill in the art are also possible, and within the scope of the present disclosure. In the case of conflict, the present specification, including definitions, will control. In addition, the materials, methods, and examples are illustrative only and not intended to be limiting.

Unless otherwise defined, scientific and technical terms used in connection with the present disclosure shall have the meanings that are commonly understood by those of ordinary skill in the art. Generally, nomenclatures utilized in connection with, and techniques of, cell and tissue culture, molecular biology, and protein and oligonucleotide or polynucleotide chemistry and hybridization described herein are those well-known and commonly used in the art (see, e.g., Green, M. and Sambrook, J. (2012) Molecular Cloning: A Laboratory Manual. 4th Edition, Vol. II, Cold Spring Harbor Laboratory Press, New York.).

As used in this specification and the appended claims, the singular forms “a”, “an” and “the” include plural references unless the content clearly dictates otherwise. Thus, for example, a composition comprising “a compound” includes a mixture of two or more compounds and the term “a cell” includes a single cell as well as a plurality or population of cells. It should also be noted that the term “or” is generally employed in its sense including “and/or” unless the content clearly dictates otherwise.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either both of those included limits are also included in the description.

As used in this application and claim(s), the word “consisting” and its derivatives, are intended to be close-ended terms that specify the presence of stated features, elements, components, groups, integers, and/or steps, and also exclude the presence of other unstated features, elements, components, groups, integers and/or steps.

In understanding the scope of the present disclosure, the term “comprising” and its derivatives, (such as “comprise” and “comprises”), “having” (and any form of having, such as “have” and “has”), “including” (and any form of including, such as “include” and “includes”) or “containing” (and any form of containing, such as “contain” and “contains”), as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives.

The terms “about”, “substantially” and “approximately” as used herein mean a reasonable amount of deviation of the modified term such that the end result is not significantly changed. These terms of degree should be construed as including a deviation of at least ±5% or at least ±10% of the modified term if this deviation would not negate the meaning of the word it modifies.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein in the specification and in the claims, the phrase “at least one,” in reference to a list of one or more element(s), should be understood to mean at least one element selected from any one or more of the element(s) in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, in certain methods described herein that include more than one step or act, the order of the steps or acts of the method is not necessarily limited to the order in which the steps or acts of the method are recited unless the context indicates otherwise.

The term “complementarity determining region” or “CDR” as used herein refers to particular hypervariable regions of antibodies that are commonly presumed to contribute to epitope binding.

The term “antigen-binding fragment” as used herein refers to a part or portion of an antibody or antibody chain comprising at least a variable domain and fewer amino acid residues than an intact or complete antibody or antibody chain and which binds the antigen or competes with intact antibody. Exemplary antigen-binding fragments include Fab, Fab′, F(ab′)2, scFv, dsFv, ds-scFv, minibodies, diabodies, and multimers thereof, fusions comprising at least two antibody domains, and single domain antibodies such as V_{H}Hs, V_{L}s, V_{H}s and variable new antigen receptors (V_{NAR}S). Fragments can be obtained via chemical or enzymatic treatment of an intact or complete antibody or antibody chain. Fragments can also be obtained by recombinant means. Antibody binding fragments may be any class of immunoglobulins including: IgG, IgM, IgD, IgA, IgY or IgE; and any isotype thereof, including IgG1, IgG2 (e.g., IgG2a, IgG2b), IgG3 and IgG4.

The term “variable domain” as used herein refers to a polypeptide that is an immunoglobulin domain comprising three complementarity determining regions along with four framework regions (i.e., FR1, FR2, FR3, and FR4).

The term “cell” as used herein refers to a single cell or a plurality of cells.

A “conservative amino acid substitution” as used herein, is one in which one amino acid residue is replaced with another amino acid residue without abolishing the protein's desired properties. Suitable conservative amino acid substitutions can be made by substituting amino acids with similar hydrophobicity, polarity, and R-chain length for one another. Examples of conservative substitutions include the substitution of one non-polar (hydrophobic) residue such as alanine, isoleucine, valine, leucine or methionine for another, the substitution of one polar (hydrophilic) residue for another such as between arginine and lysine, between glutamine and asparagine, between glycine and serine, the substitution of one basic residue such as lysine, arginine or histidine for another, or the substitution of one acidic residue, such as aspartic acid or glutamic acid for another. The phrase “conservative substitution” also includes the use of a chemically derivatized residue or non-natural amino acid in place of a non-derivatized residue provided that such polypeptide displays the requisite activity.

As used herein, the terms “peptide,” “polypeptide,” and “protein” refer to any chain of two or more natural or unnatural amino acid residues, regardless of post-translational modifications (e.g., glycosylation or phosphorylation). Included are proteins that are a single polypeptide chain and multisubunit proteins (e.g., composed of 2 or more polypeptide chains).

The term “sequence identity” as used herein refers to the percentage of sequence identity between two amino acid sequences or two nucleic acid sequences. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=[number of identical overlapping positions]/[total number of positions]×100%). The determination of percent identity between two sequences can also be accomplished using a mathematical algorithm. One non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990. BLAST nucleotide searches can be performed with the NBLAST nucleotide program parameters set, e.g., for score=100, wordlength=12 to obtain nucleotide sequences homologous to nucleic acid molecules of the present disclosure. BLAST protein searches can be performed with the XBLAST program parameters set, e.g., to score-50, wordlength=3 to obtain amino acid sequences homologous to a protein molecule of the present disclosure. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules. When utilizing BLAST, Gapped BLAST, and PSI-Blast programs, the default parameters of the respective programs (e.g., of XBLAST and NBLAST) can be used (see, e.g., the NCBI website). Another non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

The term “nucleic acid” or “nucleic acid molecule”, as used herein, are intended to include unmodified DNA or RNA or modified DNA or RNA. The nucleic acid molecules of the disclosure may contain one or more modified base(s) or DNA or RNA backbone(s) modified for stability or for other reasons. Unless otherwise indicated, standard IUPAC-IUB nomenclature is used herein. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning. The nucleic acid molecule can be either double stranded or single stranded, and represents the sense or antisense strand. Further, the term “nucleic acid molecule” includes the complementary nucleic acid sequences as well as codon optimized or synonymous codon equivalents. The term “isolated nucleic acid molecules” as used herein refers to a nucleic acid molecule substantially free of cellular material or culture medium when produced by recombinant DNA techniques, or chemical precursors, or other chemicals when chemically synthesized.

The term “vector” as used herein comprises any intermediary vehicle for a nucleic acid molecule which enables said nucleic acid molecule, for example, to be introduced into prokaryotic and/or eukaryotic cells and/or integrated into a genome, and includes plasmids, phagemids, bacteriophage or viral vectors such as retroviral based vectors, adeno-associated viral vectors and the like. The term “plasmid” as used herein generally refers to a construct of extrachromosomal genetic material, usually a circular DNA duplex, which can replicate independently of chromosomal DNA.

The term “pharmaceutically acceptable” means compatible with the treatment of animals, in particular, humans.

The definitions and embodiments described in particular sections are intended to be applicable to other embodiments herein described for which they are suitable as would be understood by a person skilled in the art.

The recitation of numerical ranges by endpoints herein includes all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

### II. Engineered Variable Domains, Antibodies, Nucleic Acids, Libraries, Cells, and Animals

An aspect of the disclosure includes an engineered immunoglobulin (Ig) variable domain comprising or capable of comprising at least one non-canonical disulfide bond wherein the at least one non-canonical disulfide bond is or can be formed between
a cysteine residue at position 24 and a cysteine residue at position 86;
a cysteine residue at position 22 and a cysteine residue at position 88;
a cysteine residue at position 4 and a cysteine residue at position 25;
a cysteine residue at position 4 and a cysteine residue at position 118;
a cysteine residue at position 5 and a cysteine residue at position 120;
a cysteine residue at position 6 and a cysteine residue at position 119; and/or
a cysteine residue at position 45 and a cysteine residue at position 100; wherein the position numbering refers to IMGT numbering.

The engineered immunoglobulin (Ig) variable domain “capable of comprising at least one non-canonical disulfide bond” refers to for example, an engineered Ig variable domain that will form or does form (e.g., can be formed, capable of forming) a disulfide bridge between two cysteine residues under suitable conditions, e.g., oxidizing conditions. In some embodiments, the engineered immunoglobulin (Ig) variable domain is capable of comprising at least one non-canonical disulfide bond wherein the at least one non-canonical disulfide bond can be formed between two cysteine residues under oxidizing conditions. Suitable oxidizing conditions can include conditions found in an oxidizing portion or organelle of a cell, such as: the periplasmic space of a prokaryotic cell, the cytoplasm of a prokaryotic cell that has been engineered to have an oxidizing cytoplasm (e.g., an E. coli strain with a trxB and/or gor mutation), or the endoplasmic reticulum or Golgi apparatus of a eukaryotic cell. Suitable oxidizing conditions can also include in vitro conditions. For example, a denatured engineered Ig variable domain may be renatured using an oxidizing renaturation solution, as will be known to one skilled in the art. Typical renaturation solutions include a suitable buffer, buffer concentration and buffer pH; a suitable concentration of the protein to be renatured; stabilizer(s), such as L-Arg; and a suitable concentration of the redox pair. A renaturation solution may also include chaperones and enzymes to help with the folding of the protein and formation of correct disulphide linkages.

In some embodiments, the at least one non-canonical disulfide bond is formed within a single framework region. In some embodiments, the at least one non-canonical disulfide bond is formed between two different framework regions.

In some embodiments, the at least one non-canonical disulfide bond is formed between a cysteine residue at position 24 and a cysteine residue at position 86. In some embodiments, the at least one non-canonical disulfide bond is formed between a cysteine residue at position 22 and a cysteine residue at position 88. In some embodiments, the at least one non-canonical disulfide bond is formed between a cysteine residue at position 4 and a cysteine residue at position 25. In some embodiments, the at least one non-canonical disulfide bond is formed between a cysteine residue at position 4 and a cysteine residue at position 118. In some embodiments, the at least one non-canonical disulfide bond is formed between a cysteine residue at position 5 and a cysteine residue at position 120. In some embodiments, the at least one non-canonical disulfide bond is formed between a cysteine residue at position 6 and a cysteine residue at position 119. In some embodiments, the at least one non-canonical disulfide bond is formed between a residue at position 45 and a cysteine residue at position 100.

In some embodiments, the variable domain is a heavy chain variable domain.

In some embodiments, the variable domain is a V_{H}H domain.

In some embodiments, the variable domain is a light chain variable domain. In some embodiments, the light chain variable domain is of the of the lambda or kappa family.

In some embodiments, the variable domain is a V_{NAR} variable domain.

It is expected that all Ig variable domains (irrespective of sequence) can be used herein, due to structural conservation of the variable Ig fold in different Ig variable domains. In some embodiments, the variable domain is human. In other embodiments, the variable domain is non-human, for example murine, rabbit, shark, camel, llama, alpaca, vicugna, guanaco or chicken.

In some embodiments, the engineered Ig variable domain comprises any one of the amino acid sequences of SEQ ID NOs: 332-338, 340-346, 348-354, 356-362, 364-370, 372-378, 380-386, 388-394, 396-402, 404-410, 412-418, 420-426, 428-434, 436-442, 444-450, 452-458, 460-466, 468-474, 476-482, 484-490, 492-498, 500-506, 508-514, 516-522, 524-530, 532-538, 540-546, 548-554, 556-562, 564-570, 572-578, 580-586, 588-594, and 596-602 or an amino acid sequence having at least 70%, at least 80%, at least 85% at least 90% or at least 95% sequence identity to any one of the amino acid sequences of SEQ ID NOs: 332-338, 340-346, 348-354, 356-362, 364-370, 372-378, 380-386, 388-394, 396-402, 404-410, 412-418, 420-426, 428-434, 436-442, 444-450, 452-458, 460-466, 468-474, 476-482, 484-490, 492-498, 500-506, 508-514, 516-522, 524-530, 532-538, 540-546, 548-554, 556-562, 564-570, 572-578, 580-586, 588-594, and 596-602, wherein the cysteine residue(s) introduced at position 24, position 86, position 22, position 88, position 4, position 25, position 118, position 5, position 120, position 6, position 119, position 45, and/or position 100 are maintained. In some embodiments, the engineered Ig variable domain comprises a conservatively substituted amino acid sequence of any one of the amino acid sequences of SEQ ID NOs: 332-338, 340-346, 348-354, 356-362, 364-370, 372-378, 380-386, 388-394, 396-402, 404-410, 412-418, 420-426, 428-434, 436-442, 444-450, 452-458, 460-466, 468-474, 476-482, 484-490, 492-498, 500-506, 508-514, 516-522, 524-530, 532-538, 540-546, 548-554, 556-562, 564-570, 572-578, 580-586, 588-594, and 596-602, wherein the cysteine residue(s) introduced at position 24, position 86, position 22, position 88, position 4, position 25, position 118, position 5, position 120, position 6, position 119, position 45, and/or position 100 are maintained. In some embodiments, the CDRs of the engineered Ig domain can be any suitable CDRs.

In some embodiments, the cysteine residue is introduced by mutating a nucleic acid molecule encoding the engineered Ig variable domain as further described below. In some embodiments, artificial gene synthesis, site-directed mutagenesis, overlap extension polymerase chain reaction (PCR), or genome editing technologies, such as CRISPR/Cas9 is used to mutate the nucleic acid molecule encoding the engineered Ig variable domain. In some embodiments, artificial gene synthesis is used to mutate the nucleic acid molecule encoding the engineered Ig variable domain.

Another aspect of the disclosure includes an antibody or antigen-binding fragment thereof comprising at least one engineered Ig variable domain as described herein.

In some embodiments, the antibody is human. In other embodiments, the antibody is non-human, for example murine, rabbit or chicken.

In some embodiments, the antibody is a single domain antibody. In some embodiments, the single domain antibody is a nanobody.

In some embodiments, the antibody is an IgG1, IgG2, IgG3, IgG4, IgE, IgA, IgD, IgY, IgM, and/or IgNAR.

In some embodiments, the antigen-binding fragment is selected from Fab, Fab′, F(ab′)2, scFv, dsFv, ds-scFv, minibody, diabody, and multimers thereof.

Another aspect of the disclosure includes a polypeptide comprising a plurality of engineered Ig variable domains as described herein. In some embodiments, the plurality comprises a plurality of the same engineered Ig variable domain. In some embodiments, the plurality comprises different engineered Ig variable domains.

In some embodiments, an antibody or antigen-binding fragment thereof as described herein, a polypeptide as described herein or an engineered Ig variable domain as described herein may be fused to an antibody fragment such as one or more than one Fc domain, Fab, Fab′, F(ab′)2, scFv, dsFv, ds-scFv, minibody, diabody, or multimers thereof. In some embodiments, an antibody or antigen-binding fragment thereof as described herein, a polypeptide as described herein or an engineered Ig variable domain as described herein may be fused to a single domain antibody, such as a V_{H}H, V_{L}, V_{H} or V_{NAR}.

In some embodiments, an antibody or antigen-binding fragment thereof as described herein, a polypeptide as described herein, or an engineered Ig variable domain as described herein may be fused or conjugated to a cargo molecule, optionally via a linker or a chelator. The cargo molecule may be, for example, a diagnostic or therapeutic agent, which are known in the art. For example, a therapeutic agent may be a radioisotope, which may be used for radioimmunotherapy; a toxin, such as an immunotoxin; a cytokine, such as an immunocytokine; a cytotoxin; an apoptosis inducer; an enzyme; or any other suitable therapeutic molecule known in the art. A diagnostic agent may include, for example, a radioisotope, a paramagnetic label, a fluorophore, an affinity label, fluorescent protein or any other suitable agent that may be detected by imaging methods.

Another aspect of the disclosure includes a nucleic acid molecule encoding an engineered Ig variable domain as described herein, an antibody or antigen-binding fragment thereof as described herein, or a polypeptide as described herein. The nucleic acid molecule may be comprised in a vector. The nucleic acid molecule may, for example, be incorporated into an expression cassette or expression vector for expression of the antibody or antigen-binding fragment thereof. Accordingly, an aspect of the present disclosure includes an expression cassette or a vector, optionally an expression vector, comprising a nucleic acid molecule as described herein. Suitable expression vectors include, but are not limited to, cosmids, plasmids, or modified viruses (e.g., replication defective retroviruses, adenoviruses and adeno-associated viruses).

Another aspect of the disclosure is a library comprising a plurality of nucleic acid molecules or vectors as described herein. In an embodiment, the nucleic acid molecules or vectors in the library may comprise different engineered cysteine residues, while otherwise having the same sequence. Such a library may be used, for example, to screen for disulfide bonds that increase stability of an engineered Ig variable domain as described herein, an antibody or antigen-binding fragment thereof as described herein, or a polypeptide as described herein. In another embodiment, the nucleic acid molecules or vectors in the library may encode the same engineered cysteine residues, but encode different framework and/or CDR sequences. Such a library may, for example, be used to screen for an Ig variable domain as described herein, an antibody or antigen-binding fragment thereof as described herein, or a polypeptide as described herein that binds an antigen or target of interest. In a particular embodiment, the library may be a phage display library.

Another aspect of the disclosure includes a vector comprising a nucleic acid molecule as described herein.

Another aspect of the disclosure includes a cell expressing an engineered Ig variable domain as described herein, an antibody or antigen-binding fragment thereof as described herein, a polypeptide as described herein, a nucleic acid molecule as described herein, or a vector or expression cassette as described herein. The engineered cell can be prepared by introducing the nucleic acid molecule, expression cassette, or vector into a suitable host cell. Preferably, the host cell is suitable for producing large quantities of the expression cassette or the vector. As would be known to the skilled artisan, a vector compatible with the particular host cell is used.

The engineered cell can be generated using any host cell suitable for producing a polypeptide, for example suitable for producing an engineered Ig variable domain, an antibody and/or an antigen-binding fragment thereof. For example, to introduce a nucleic acid molecule and/or a vector into a cell, the host cell may be transfected, transformed or infected, depending upon the vector employed. Suitable host cells include a wide variety of prokaryotic and eukaryotic host cells. For example, engineered Ig variable domains and antibodies and antigen-binding fragments thereof as described herein may be expressed in bacterial cells such as E. coli, insect cells (using baculovirus), yeast cells or mammalian cells. In some embodiments, the engineered cell is a human cell. In some embodiments, the engineered cell is a HEK cell. In some embodiments, the engineered cell is a CHO cell. The engineered cell may be used, for example, for in vitro production of an engineered Ig variable domain as described herein, an antibody or antigen-binding fragment thereof as described herein, or a polypeptide as described herein.

Another aspect of the disclosure includes an animal engineered to comprise VDJ gene segments comprising a nucleic acid molecule encoding an engineered Ig variable domain as described herein, an antibody or antigen-binding fragment thereof as described herein, or a polypeptide as described herein, which allows the animal to express the engineered Ig variable domain, the antibody or antigen-binding fragment thereof, or the polypeptide. The animal may be any species into which VDJ gene segments comprising the nucleic acid molecule may be introduced, for example by transgenesis or genetic engineering. Suitable animals include, for example, mice, rats, chickens, rabbits, cattle, pigs, sheep, and goats. In some embodiments, the animal may be used to produce heterologous antibodies, or antigen-binding fragments thereof, such as human, humanized, or camelid antibodies or antigen-binding fragments thereof.

Another aspect of the disclosure includes an engineered library comprising at least one nucleic acid molecule encoding at least one engineered Ig variable domain or at least one antibody or antigen-binding fragment thereof as described herein. In some embodiments, the at least one engineered Ig variable domain or the at least one antibody or antigen-binding fragment thereof is a plurality and at least a subset of the plurality comprises different CDR sequences. In some embodiments, the at least one engineered Ig variable domain is part of at least one antibody binding fragment, such as Fab, Fab′, F(ab′)2, scFv, dsFv, ds-scFv, minibody, diabody, or multimers thereof or a single domain antibody such as V_{H}, V_{L}, V_{H}H, or V_{NAR}.

In some embodiments, the engineered library is a phage display library. In some embodiments, the library has a diversity of a library in Table 1. In some embodiments, the library is a library as described in Example 1. In some embodiments, the engineered library is made using mutagenesis, optionally Kunkel mutagenesis. In some embodiments, the engineered library is made using the method described in Example 1. In some embodiments, the engineered library is a ribosome display library. In some embodiments, the engineered library is a yeast display library. In some embodiments, the engineered library is a mammalian cell display library. In some embodiments, the engineered library is a bacterial display library. In some embodiments, the engineered library is a V_{H}H library. In some embodiments, the engineered library is an Ig light chain variable domain library. In some embodiments, the engineered library is an Ig heavy chain variable domain library.

In some embodiments, the library is generated using an antibody display technology. In some embodiments, the library is generated using phage display. In some embodiments, the library is generated using ribosome display. In some embodiments, the library is generated using yeast display. In some embodiments, the library is generated using mammalian cell display. In some embodiments, the library is generated using bacterial display.

In some embodiments, the library is generated using Kunkel mutagenesis. In some embodiments, the library is generated according to the methods provided in the Examples. In some embodiments, the library is generated using trinucleotide mutagenesis, PCR with degenerate oligonucleotides, random error-prone PCR mutagenesis, recombination, or any other methods known in the art for library generation.

An engineered Ig variable domain as described herein can be considered a variable domain scaffold for preparing desired stabilized antibodies. For example, the engineered variable domain, i.e., scaffold, can be used to prepare specific antibodies by grafting in desired CDRs.

### III. Compositions

Another aspect of the disclosure includes a composition comprising an engineered Ig variable domain as described herein, an antibody or antigen-binding fragment thereof as described herein, a polypeptide as described herein, a nucleic acid molecule as described herein, or a vector or expression cassette as described herein and optionally a pharmaceutically acceptable carrier and/or excipient.

In an embodiment the composition comprises a diluent. Suitable diluents for nucleic acid molecules include but are not limited to water, saline solutions and ethanol. Suitable diluents for polypeptides, including engineered Ig variable domains, antibodies or fragments thereof and/or cells, include but are not limited to saline solutions, pH buffered solutions and glycerol solutions or other solutions suitable for freezing polypeptides and/or cells.

The composition may be formulated for use or prepared for administration to a subject using pharmaceutically acceptable formulations known in the art. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999.

The compositions described herein can be prepared by per se known methods for the preparation of pharmaceutically acceptable compositions that can be administered to subjects such that an effective quantity of the active substance is combined in a mixture with a pharmaceutically acceptable vehicle.

Pharmaceutical compositions include, without limitation, lyophilized powders or aqueous or non-aqueous sterile injectable solutions or suspensions, which may further contain antioxidants, buffers, bacteriostats and solutes that render the compositions substantially compatible with the tissues or the blood of an intended recipient. Other components that may be present in such compositions include, for example, water, surfactants (such as Tween), alcohols, polyols, glycerin and vegetable oils. Extemporaneous injection solutions and suspensions may be prepared from sterile powders, granules, tablets, or concentrated solutions or suspensions. The composition may be supplied, for example but not by way of limitation, as a lyophilized powder which is reconstituted with sterile water or saline prior to administration to a patient.

### IV. Methods

Another aspect of disclosure includes a method of making an antibody or antigen-binding fragment thereof with increased stability, the method comprising generating an antibody or antigen-binding fragment thereof comprising at least one of the engineered Ig variable domains described herein.

In some embodiments, the method comprises substituting one or more than one amino acid residue in a variable domain at position 24, 86, 22, 88, 4, 25, 118, 5, 120, 6, 119, 45, and/or 100, based on IMGT numbering, with a cysteine residue, wherein said substituting comprises providing a polynucleotide encoding an amino acid sequence of the modified antibody in which the one or more than one amino acid residue at position 24, 86, 22, 88, 4, 25, 118, 5, 120, 6, 119, 45, and/or 100 is a cysteine residue; and producing the antibody with increased stability.

In some embodiments, the antibody or antigen-binding fragment thereof is generated by expressing a nucleic acid molecule encoding the at least one engineered Ig variable domain described herein. In some embodiments, the nucleic acid molecule encoding the at least one engineered Ig variable domain described herein is generated using Kunkel mutagenesis. In some embodiments, the nucleic acid molecule encoding the at least one engineered Ig variable domain described herein is generated using the methods described in the Examples. In some embodiments, the nucleic acid molecule encoding the at least one engineered Ig variable domain described herein comprises a cysteine residue introduced by mutating a nucleic acid molecule encoding the engineered Ig variable domain as further described below. In some embodiments, artificial gene synthesis, site-directed mutagenesis, overlap extension PCR, or a genome editing technology, such as CRISPR/Cas9, is used to mutate the nucleic acid molecule encoding the engineered Ig variable domain. In some embodiments, artificial gene synthesis is used to mutate the nucleic acid molecule encoding the at least one engineered Ig variable domain.

Another aspect of the disclosure includes a method of increasing stability of an antibody or antigen-binding fragment thereof, the method comprising introducing into at least one variable domain of the antibody or antigen-binding fragment thereof
a cysteine residue at position 24 and a cysteine residue at position 86;
a cysteine residue at position 22 and a cysteine residue at position 88;
a cysteine residue at position 4 and a cysteine residue at position 25;
a cysteine residue at position 4 and a cysteine residue at position 118;
a cysteine residue at position 5 and a cysteine residue at position 120;
a cysteine residue at position 6 and a cysteine residue at position 119; and/or
a cysteine residue at position 45 and a cysteine residue at position 100,
   wherein the position numbering refers to IMGT numbering, and wherein the cysteine residue(s) introduced form or are capable of forming a disulfide bond.

Another aspect of the disclosure includes a method of increasing stability of an antibody or antigen-binding fragment thereof, the method comprising introducing into at least one variable domain of the antibody or antigen-binding fragment thereof a first cysteine and a second cysteine, wherein the first and second cysteines are selected from a cysteine residue introduced at position 24, position 86, position 22, position 88, position 4, position 25, position 118, position 5, position 120, position 6, position 119, position 45, and/or position 100, wherein the position numbering refers to IMGT numbering, wherein the first and second cysteines are not at the same position, and wherein the cysteine residue(s) introduced form or are capable of forming a disulfide bond.

In some embodiments, the at least one non-canonical disulfide bond is formed between a cysteine residue at position 24 and a cysteine residue at position 86. In some embodiments, the at least one non-canonical disulfide bond is formed between a cysteine residue at position 22 and a cysteine residue at position 88. In some embodiments, the at least one non-canonical disulfide bond is formed between a cysteine residue at position 4 and a cysteine residue at position 25. In some embodiments, the at least one non-canonical disulfide bond is formed between a cysteine residue at position 4 and a cysteine residue at position 118. In some embodiments, the at least one non-canonical disulfide bond is formed between a cysteine residue at position 5 and a cysteine residue at position 120. In some embodiments, the at least one non-canonical disulfide bond is formed between a cysteine residue at position 6 and a cysteine residue at position 119. In some embodiments, the at least one non-canonical disulfide bond is formed between a residue at position 45 and a cysteine residue at position 100.

In some embodiments, the variable domain is a heavy chain variable domain.

In some embodiments, the variable domain is a V_{H}H domain.

In some embodiments, the variable domain is a light chain variable domain. In some embodiments, the light chain variable domain is of the of the lambda or kappa family.

In some embodiments, the variable domain is a human variable domain. In other embodiments, the variable domain is non-human, for example murine, rabbit or chicken.

In some embodiments, the engineered Ig variable domain comprises any one of the amino acid sequences of SEQ ID NOs: 332-338, 340-346, 348-354, 356-362, 364-370, 372-378, 380-386, 388-394, 396-402, 404-410, 412-418, 420-426, 428-434, 436-442, 444-450, 452-458, 460-466, 468-474, 476-482, 484-490, 492-498, 500-506, 508-514, 516-522, 524-530, 532-538, 540-546, 548-554, 556-562, 564-570, 572-578, 580-586, 588-594, and 596-602 or an amino acid sequence having at least 50%, at least 60%, at least 70%, at least 80%, at least 85% at least 90% or at least 95% sequence identity to any one of the amino acid sequences of SEQ ID NOs: 332-338, 340-346, 348-354, 356-362, 364-370, 372-378, 380-386, 388-394, 396-402, 404-410, 412-418, 420-426, 428-434, 436-442, 444-450, 452-458, 460-466, 468-474, 476-482, 484-490, 492-498, 500-506, 508-514, 516-522, 524-530, 532-538, 540-546, 548-554, 556-562, 564-570, 572-578, 580-586, 588-594, and 596-602, wherein the cysteine residue(s) introduced at position 24, position 86, position 22, position 88, position 4, position 25, position 118, position 5, position 120, position 6, position 119, position 45, and/or position 100 are maintained. In some embodiments, the engineered Ig variable domain comprises a conservatively substituted amino acid sequence of any one of the amino acid sequences of SEQ ID NOs: 332-338, 340-346, 348-354, 356-362, 364-370, 372-378, 380-386, 388-394, 396-402, 404-410, 412-418, 420-426, 428-434, 436-442, 444-450, 452-458, 460-466, 468-474, 476-482, 484-490, 492-498, 500-506, 508-514, 516-522, 524-530, 532-538, 540-546, 548-554, 556-562, 564-570, 572-578, 580-586, 588-594, and 596-602, wherein the cysteine residue(s) introduced at position 24, position 86, position 22, position 88, position 4, position 25, position 118, position 5, position 120, position 6, position 119, position 45, and/or position 100 are maintained. In some embodiments, the CDRs of the engineered Ig domain can be any suitable CDRs.

In some embodiments, the antibody or antigen-binding fragment thereof has increased thermostability and/or increased resistance to protease activity as compared to a control antibody or antigen-binding fragment. In some embodiments, the control is the same antibody or antigen-binding fragment comprising a non-engineered Ig variable domain (i.e., the control does not have the non-canonical disulfide bonds described herein introduced therein but is otherwise the same as the engineered antibody or antigen-binding fragment thereof).

Another aspect of the disclosure includes a method for identifying one or more location(s) in engineered immunoglobulin (Ig) variable domains tolerant of disulfide bonds, the method comprising
generating a library of nucleic acid molecules encoding engineered immunoglobulin (Ig) variable domains lacking a disulfide bond between a cysteine residue at position 23 and a cysteine residue at position 104, wherein at least one cysteine residue has been introduced into the amino acid sequence of each engineered Ig variable domain encoded by the nucleic acid molecules of the library;
identifying Ig variable domains encoded by the nucleic acid molecules of the library which have formed at least one disulfide bond; and
identifying the positions between which the at least one disulfide bond is formed in each engineered Ig variable domain,
   wherein the position numbering refers to IMGT numbering.

In some embodiments, the Ig variable domain is a heavy chain variable domain. In some embodiments, the Ig variable domain is a light chain variable domain. In some embodiments, the Ig variable domain is a V_{H}H domain.

In some embodiments, the engineered Ig variable domain is part of at least one antibody binding fragment, such as Fab, Fab′, F(ab′)2, scFv, dsFv, ds-scFv, minibody, diabody, and multimers thereof or a single domain antibody such as V_{H}, V_{L}, V_{H}H, or V_{NAR}.

Another aspect of the disclosure includes a method of identifying engineered immunoglobulin (Ig) variable domain disulfide bonds that increase stability or solubility of the Ig variable domain, the method comprising:
generating a library of nucleic acid molecules encoding engineered immunoglobulin (Ig) variable domains lacking a disulfide bond between a cysteine residue at position 23 and a cysteine residue at position 104, wherein engineered pairs of cysteine residues have been introduced into the amino acid sequence of each engineered Ig variable domain encoded by the nucleic acid molecules of the library;
identifying Ig variable domains encoded by the nucleic acid molecules of the library which have formed at least one disulfide bond,
determining the stability or solubility of the identified Ig variable domains, and
identifying the positions between which the disulfide bonds are formed in each engineered Ig variable domain that has increased stability or solubility as compared to a control,
wherein the position numbering refers to IMGT numbering.

In some embodiments, the engineered Ig variable domains are part of at least one antibody binding fragment, such as Fab, Fab′, F(ab′)2, scFv, dsFv, ds-scFv, minibody, diabody, or multimers thereof or a single domain antibody such as V_{H}, V_{L}, V_{H}H, or V_{NAR}.

In some embodiments, the library is generated using an antibody display technology. In some embodiments, the library is generated using phage display. In some embodiments, the library is generated using ribosome display. In some embodiments, the library is generated using yeast display. In some embodiments, the library is generated using mammalian cell display. In some embodiments, the library is generated using bacterial display.

In some embodiments, the library is generated using Kunkel mutagenesis. In some embodiments, the library is generated according to the methods provided in the Examples. In some embodiments, the library is generated using trinucleotide mutagenesis, PCR with degenerate oligonucleotides, random error-prone PCR mutagenesis, recombination, or any other methods known in the art for library generation.

In some embodiments, the engineered library is a V_{H}H library. In some embodiments, the engineered library is an Ig light chain variable domain library. In some embodiments, the engineered library is an Ig heavy chain variable domain library. In some embodiments, the engineered library is an antibody binding fragment library, comprising nucleic acid molecules encoding antibody binding fragments such as Fab, Fab′, F(ab′)2, scFv, dsFv, ds-scFv, minibodies, diabodies, and multimers thereof. In some embodiments, the engineered library is a single domain antibody library, comprising nucleic acid molecules encoding single domain antibodies such as V_{H}S, V_{L}s, V_{H}Hs, or V_{NAR}s.

In some embodiments, the Ig variable domains encoded by the nucleic acid molecules of the library which have formed at least one disulfide bond other than at positions 23 and 104 are identified using panning of the library. In some embodiments, the Ig variable domains encoded by the nucleic acid molecules of the library which have formed at least one disulfide bond other than at positions 23 and 104 are identified according to the methods provided in the Examples. In some embodiments, the Ig variable domains encoded by the nucleic acid molecules of the library which have formed at least one disulfide bond other than at positions 23 and 104 are identified using negative selection to negatively select molecules with free Cys residues and/or molecules that form intermolecular S-S linkages (dimers). In some embodiments, the Ig variable domains encoded by the nucleic acid molecules of the library which have formed at least one disulfide bond other than at positions 23 and 104 are identified by screening all the generated Ig variable domains individually using high-throughput screening.

In some embodiments, the positions between which the disulfide bonds are formed are identified using next generation sequencing. In some embodiments, the positions between which the disulfide bonds are formed are identified according to the methods provided in the Examples.

In some embodiments, the stability is determined by measuring thermostability of the engineered Ig variable domain. In some embodiments, the stability is determined by measuring protease resistance. In some embodiments, stability is measured according to the methods provided in the Examples. In some embodiments, solubility is measured accordingly to the methods provided in the Examples.

In some embodiments, the Ig variable domain is a heavy chain variable domain. In some embodiments, the Ig variable domain is a light chain variable domain. In some embodiments, the Ig variable domain is a V_{H}H domain.

### V. Kits

Another aspect of the disclosure includes a kit comprising a engineered Ig variable domain as described herein, a antibody or antigen-binding fragment thereof as described herein, a polypeptide as described herein, a nucleic acid molecule as described herein, a vector or expression cassette as described herein and/or a composition as described herein. In some embodiments, the kit further comprises packaging material (e.g., one or more vial(s)) and/or instructions for using the components included in the kit.

The definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature indicated as being preferred or advantageous may be combined with any other feature or features, including those indicated as being preferred or advantageous.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

The following non-limiting examples are illustrative of the present disclosure:

### EXAMPLES

### Example 1

Based on the serendipitous discovery of non-canonical disulfide linkages within Ig V domains in the past and an increasing acknowledgement of their importance in structural diversification of Ig repertoires (15,16), it was hypothesized that formation of such linkages at additional locations within Ig V domain FRs would be compatible with antigen binding. To investigate this possibility, an unbiased library approach was applied to identify Ig V domain FR positions permissive for non-canonical disulfide linkage formation. Surprisingly, it was discovered that diverse intra- and inter-strand disulfide linkages that are absent in the natural human repertoire could be accommodated within Ig V domain FRs, improving the stability of these domains without compromising antigen binding.

Starting from a destabilized human V_{H} domain lacking the canonical Cys23-Cys104 disulfide linkage, phage-displayed libraries of engineered V_{H}s bearing all possible pairwise combinations of Cys residues in neighboring β-strands of the Ig fold FRs were generated and screened. This approach identified seven novel Cys pairs in V_{H} FRs (Cys24-Cys86, Cys22-Cys88, Cys4-Cys25, Cys4-Cys118, Cys5-Cys120, Cys6-Cys119 and Cys45-Cys100 by IMGT numbering) whose presence putatively resulted in formation of non-canonical disulfide linkages (four intra- and three inter-β sheet), rescuing domain folding and stability. Introduction of a subset of these putative disulfide linkages (Cys 22-Cys88, Cys24-Cys86, Cys4-Cys25 and Cys6-Cys119) into a diverse panel of V_{H}, V_{L} and V_{H}H domains enhanced their thermostability and protease resistance without significantly impacting expression, solubility, or binding to cognate antigens. None of the non-canonical disulfide linkages were present in the natural human V_{H} repertoire. These data reveal an unexpected permissiveness of Ig V domains to non-canonical disulfide linkages at diverse locations in FRs, absent in the human repertoire, whose presence is compatible with antigen recognition and improves domain biophysical properties. This represents the most complete assessment to date of the role of engineered non-canonical disulfide bonding within FRs in Ig V domain structure and function.

### Results

### Selection of V_{H} Domain for Cys Pair Scanning.

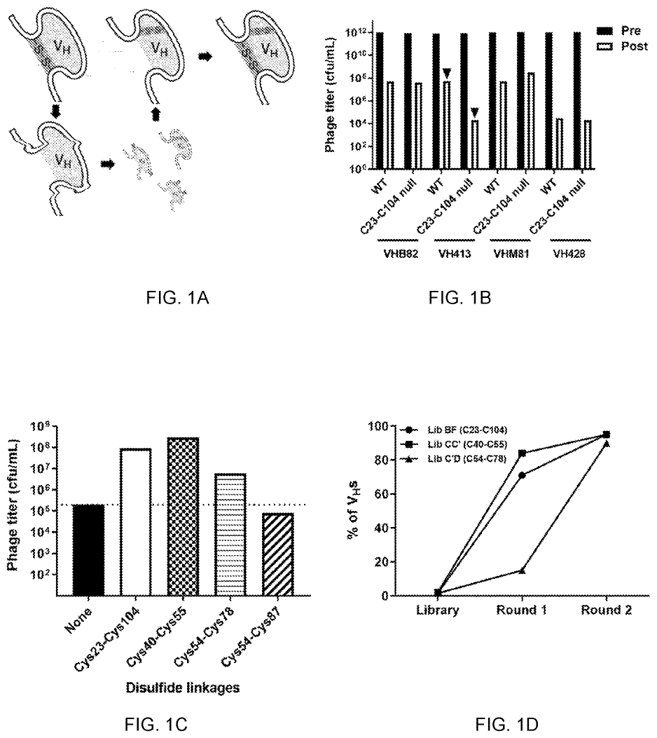

To identify novel non-canonical disulfide linkages within Ig V domains, a model system was needed in which domain folding was perturbed by abrogation of the canonical Cys23-Cys104 disulfide linkage (Cys23-Cys104 null) and which could be rescued by the stabilizing effects of non-canonical disulfide linkage formation elsewhere in the domain (FIG. 1A). Importantly, restoration of domain stability must be associated with a phenotype selectable by panning of phage-displayed libraries of Ig V domains such as reactivity with bacterial Ig-binding proteins, e.g., protein L, protein A (17, 18). V_{H} domains were deemed suitable for this purpose as the protein A binding site is a conformational epitope on V_{H} domains formed from at least four different β-strands in FR1 and FR3 (18); thus, binding of unfolded or partially folded domains (i.e., Cys23-Cys104 null V_{H}S with non-stabilizing non-canonical Cys pairs) cannot occur. As a result, such domains would be de-selected following panning of Cys pair scan V_{H} domain libraries on protein A, while folded domains (i.e., Cys23-Cys104 null V_{H}S containing stabilizing non-canonical Cys pairs) would be selected.

Fd phage displaying four different V_{H}S (VHB82, VH13, VHM81 and VH428; 19,20) and their Cys23-Cys104 null derivatives were generated. Following selection for binding to protein A, phage displaying the Cys23-Cys104 null variants of two of the V_{H}s had equivalent titers to those displaying the wild-type (WT) V_{H}s, indicating no loss of protein A binding in the destabilized V_{H}S. Surprisingly, both the WT and Cys23-Cys104 null variant of VH428 was inefficiently selected by panning on protein A. However, the output titers of VH413-displaying phage were 3-4 logs higher than those of VH413^{C23-C104 null}-displaying phage following protein A selection (FIG. 1B), reflecting loss of stability and folding in the destabilized V_{H}. Introduction of the well-characterized Cys54-Cys78 and Cys40-Cys55 non-canonical disulfide linkages, but not a negative control Cys pair at a location non-permissive to disulfide linkage formation (Cys54-Cys87), into phage-displayed VH413^{C23-C104 null} restored the titers of phage eluted from protein A selections to near-WT levels (FIG. 1C).

As proof-of-concept that VH413^{C23-C104 null} could be used to identify novel non-canonical disulfide linkages in Ig V domains, three test Cys pair scan libraries of phage-displayed VH413^{C23-C104 null} domains bearing all possible combinations of Cys pairs spanning β-strands B-F, C-C′ and C′-D were first constructed. Panning of the libraries rapidly enriched for VH413^{C23-C104 null} domains bearing previously identified stabilizing non-canonical disulfide linkages spanning Cys23-Cys104 (B-F), Cys40-Cys55 (C-C′) and Cys54-Cys78 (C′-D) (FIG. 1D). Efficient selection of stabilized V_{H}S from these libraries confirmed that introduction of both canonical and non-canonical disulfide linkages could rescue folding of the VH413^{C23-C104 null} domain and validated this strategy for discovery of novel non-canonical disulfide linkages.

### Design and Screening of V_{H} Cys Pair Scan Libraries.

To identify novel Ig V domain disulfide linkages without a priori assumptions of their locations, an additional 14 phage-displayed VH413^{C23-C104 null} libraries (distinct from the three test libraries described above) were constructed, each comprising mutant V_{H}S bearing all possible combinations of Cys pairs in adjoining or opposing β-strands. In seven libraries the V_{H}S bore Cys pairs on adjoining β-strands that were putatively capable of forming intra-β-sheet disulfide linkages, and in 10 libraries the V_{H}S bore Cys pairs on opposing β-strands that were putatively capable of forming inter-β-sheet disulfide linkages (Table 1). Sequencing of the libraries revealed that 70% of clones displayed V_{H}S mutagenized as intended, while 14% displayed V_{H}s bearing a single unpaired Cys and 16% displayed unmodified VH413^{C23-C104 null} Comparison of library size versus theoretical diversity values revealed that for all 14 libraries, each Cys pair clone was present multiple times in its respective library. Cys pairs in the libraries were generally evenly distributed across all targeted positions except for library C″-D, in which Cys residues at the N-terminus of strand C″ and the C-terminus of strand D predominated.

TABLE 1
Diversity of 17 Cys pair scan phage-displayed V_{H} libraries

| Intra-β- | | | Intra-β- | | |
|---|---|---|---|---|---|
| sheet | Theoretical | Library | sheet | Theoretical | Library |
| libraries | diversity¹ | size² | libraries | diversity¹ | size² |
| A-F | 112 | 5.1 × 10³ | A-B | 154 | 1.9 × 10⁴ |
| A-G | 154 | 4.5 × 10³ | B-E | 132 | 1.8 × 10⁴ |
| B-C | 88 | 6.3 × 10³ | E-D | 120 | 1.6 × 10⁴ |
| B-F | 88 | 1.9 × 0⁴ | C-C′ | 72 | 1.9 × 10⁴ |
| E-F | 96 | 8.5 × 10³ | C′-C″ | 72 | 6.4 × 10³ |
| E-C | 96 | 8.3 × 10³ | C-F | 64 | 4.6 × 10³ |
| E-C′ | 108 | 9.5 × 10³ | F-G | 88 | 8.3 × 10³ |
| D-C | 80 | 1.8 × 10⁴ | | | |
| C′-D | 90 | 3.2 × 10³ | | | |
| C″-D | 80 | 3.9 × 10³ | | | |

¹Total number of Cys pair combinations calculated based on the number of residues present on the β-strands.
²Calculated as cfu × % of clones correctly mutagenized.

The three test libraries described above (B-F, C-C′ and C′-D) were not included in library selections to prevent dominant selection of V_{H}S bearing Cys23-Cys104, Cys54-Cys78 and Cys40-Cys55 disulfide linkages. The remaining six intra-β-sheet and eight inter-β-sheet libraries were pooled separately and panned on protein A. Panning of the six intra-β-sheet libraries was conducted both at room temperature and with a 30-min phage incubation at 50 °C at the beginning of each round, while panning of the eight inter-β-sheet libraries was conducted only with the additional heating step. Sequencing of clones from the second, third and fourth rounds of both pannings revealed enrichment of V_{H}S bearing five putative intra-β-sheet disulfide linkages (Cys4-Cys25, Cys19-Cys91, Cys22-Cys88, Cys24-Cys86 and Cys45-Cys 00) and three putative inter-β-sheet disulfide linkages Cys4-Cys18, Cys5-Cys2 and Cys6-Cys119). In early panning rounds, V_{H} bearing Cys pairs at other positions were identified at low frequencies, but in most cases disulfide linkage formation was structurally implausible (Tables 2 and 3).

TABLE 2
Output of panning the 6 intra-β-sheet VH413^{C23-C104 null}
Cys pair scan libraries.

| | | Round 2 (%) | | Round 3 (%) | | Round 4 (%) | |
|---|---|---|---|---|---|---|---|
| Cys | | No | Heat | No | Heat | No | Heat |
| pair | Library | heat | (50 °C) | heat | (50 °C) | heat | (50 °C) |
| 4-25 | A-B | 2.5 | — | — | — | — | — |
| 13-20^{a} | A-B | — | 2.6 | — | — | — | — |
| 16-87^{a} | B-E | — | 2.6 | — | — | — | — |
| 19-91 | B-E | — | 2.6 | — | — | — | — |
| 22-88 | B-E | — | 34.2 | — | 23.7 | — | 100 |
| 24-86 | B-E | 97.5 | 50 | 100 | 71.1 | 100 | — |
| 45-100 | C-F | — | 2.6 | — | — | — | — |
| 47-72^{a} | C′-C″ | — | — | — | 5.2 | — | — |
| 51-66^{a} | C′-C″ | — | 2.6 | — | — | — | — |
| 83-91^{a} | D-E | — | 2.6 | — | — | — | — |

^{a}Disulfide linkage formation not structurally plausible.
Cys pairs shown in bold were deemed structurally plausible and were introduced into sdAbs and the resulting proteins characterized.
“—” means these Cys pairs were not observed following sequencing of 40 to 50 clones per panning round.

TABLE 3
Output of panning the 8 inter-β-sheet
VH413^{C23-C104 null} Cys pair scan libraries.

| | | Round 2 (%) | | Round 3 (%) | | Round 4 (%) | |
|---|---|---|---|---|---|---|---|
| Cys | | No | Heat | No | Heat | No | Heat |
| pair | Library | heat | (50 °C)^{b} | heat | (50 °C) | heat | (50 °C) |
| 4-118 | A-G | n.d. | 25 | n.d. | 61.8 | n.d. | 25 |
| 5-120 | A-G | n.d. | 5 | n.d. | — | n.d. | 2.8 |
| 6-119 | A-G | n.d. | 5 | n.d. | 29.4 | n.d. | 69.4 |
| 4-101^{a} | A-F | n.d. | — | n.d. | — | n.d. | 2.8 |
| 12-127^{b} | A-G | n.d. | 2.5 | n.d. | — | n.d. | — |
| 13-100^{a} | A-F | n.d. | 2.5 | n.d. | — | n.d. | — |
| 16-46^{a} | B-C | n.d. | 5 | n.d. | — | n.d. | — |
| 18-46^{a} | B-C | n.d. | 7.5 | n.d. | — | n.d. | — |
| 24-40^{a} | B-C | n.d. | 2.5 | n.d. | — | n.d. | — |
| 26-45^{a} | B-C | n.d. | 2.5 | n.d. | — | n.d. | — |
| 26-46^{a} | B-C | n.d. | 2.5 | n.d. | — | n.d. | — |
| 37-79^{a} | C-D | n.d. | 2.5 | n.d. | — | n.d. | — |
| 39-87^{c} | C-E | n.d. | — | n.d. | 8.8 | n.d. | — |
| 38-76^{a} | C-D | n.d. | 2.5 | n.d. | — | n.d. | — |
| 41-80^{a} | C-D | n.d. | 2.5 | n.d. | — | n.d. | — |
| 43-75^{b} | C-D | n.d. | 2.5 | n.d. | — | n.d. | — |
| 43-77^{a} | C-D | n.d. | 2.5 | n.d. | — | n.d. | — |
| 45-84^{a} | C-D | n.d. | 2.5 | n.d. | — | n.d. | — |
| 46-76^{a} | C-D | n.d. | 2.5 | n.d. | — | n.d. | — |
| 46-79^{a} | C-D | n.d. | 2.5 | n.d. | — | n.d. | — |
| 46-84^{a} | C-D | n.d. | 2.5 | n.d. | — | n.d. | — |
| 46-90^{a} | C-E | n.d. | 2.5 | n.d. | — | n.d. | — |
| 46-92^{a} | C-E | n.d. | 10 | n.d. | — | n.d. | — |
| 47-91^{a} | C′-E | n.d. | 2.5 | n.d. | — | n.d. | — |
| 47-95^{a} | C′-E | n.d. | 2.5 | n.d. | — | n.d. | — |

^{a}Disulfide linkage formation not structurally plausible.
^{b}While disulfide linkage formation may be possible at these positions, they were not tested further due to resource limitations.
^{c}Previously known Cys39-Cys87 stabilizing disulfide linkage (Saerens et al., 2008, J. Mol. Biol. 377: 478-488).
Cys pairs shown in bold were deemed structurally plausible and were introduced into sdAbs and the resulting proteins characterized, except for those noted with footnote b.
“—” means these Cys pairs were not observed following sequencing of 40 to 50 clones per panning round.
n.d., not determined.

### Effects of Novel Disulfide Linkages on Ig V Domain Biophysical Properties.

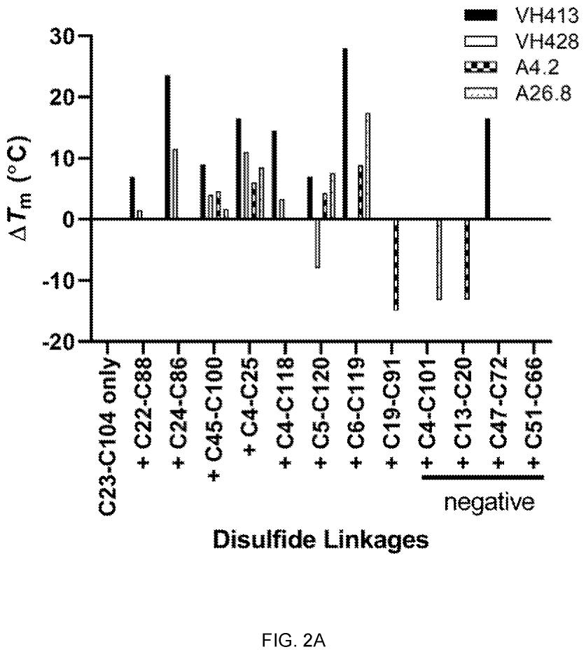

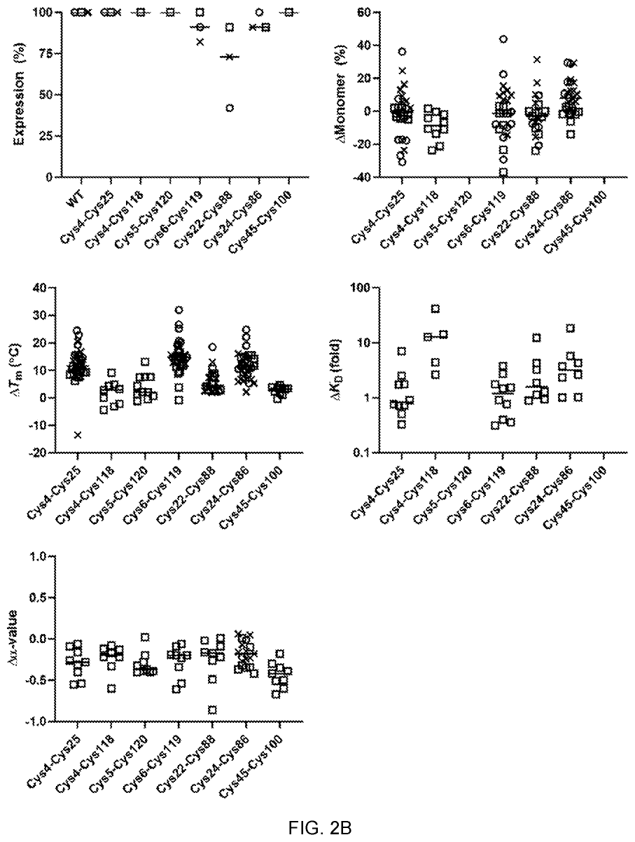

As an initial screen, first assessed were the effects of introducing the eight putative non-canonical disulfide linkages, along with several negative control Cys pairs predicted not to form disulfide linkages, on the biophysical properties of two V_{H}s (VH413 and VH428) and their Cys23-Cys104 null variants as well as two V_{H}Hs (A4.2 and A26.8; 21). VH413^{C23-C104 null} and VH428^{C23-C104 null} could not be expressed in significant amounts, but introduction of Cys24-Cys86 and, surprisingly, the negative control Cys pair Cys47-Cys72, resulted in low expression of V_{H}S with melting temperatures (Tₘs) between 40-50 °C (data not shown). In this initial screen, introduction of seven of eight putative non-canonical disulfide linkages into V_{H}s and V_{H}Hs bearing the canonical Cys24-Cys104 disulfide linkage resulted in Tₘ increases ranging from 1.5-28 °C by differential scanning fluorometry (FIG. 2A). Exceptions included Cys19-Cys91, which resulted in no expression or decreased Tₘ in all cases, and Cys5-Cys120, which resulted in decreased Tₘ for one V_{H}. As expected, negative control Cys pairs generally resulted in loss of V_{H}/V_{H}H expression or decreased Tₘ.

Based on these results, seven putative non-canonical disulfide linkages (Cys4-Cys25, Cys22-Cys88, Cys24-Cys86, Cys45-Cys100, Cys4-Cys118, Cys5-Cys120 and Cys6-Cys119) were introduced into a larger set of 12 V_{H}S, 11 V_{L}s and 11 V_{H}Hs. Three of the putative disulfide linkages (Cys45-100, Cys4-118, and Cys5-120) were introduced into only the set of 11 V_{H}Hs. The V_{H}s and V_{L}S were non-antigen-specific (19, 20, 22) while the V_{H}Hs targeted Clostridioides difficile toxins A and B (21, 23), epidermal growth factor receptor (EGFR) (24), insulin-like growth factor 1 receptor (IGF1R) (25). Introduction of all seven putative non-canonical disulfide linkages was generally well tolerated by most Ig V domains, except for Cys22-Cys88 which was successfully introduced into only 8/11 V_{L}s and 5/12 V_{H}s (FIG. 2B and Table 4). Three of the putative non-canonical disulfide linkages had only minor effects on Tₘ (Cys45-Cys100, Cys4-Cys118, Cys5-Cys120, average ΔTₘ 2.7 °C, 1.5 °C, and 4.2 °C, respectively by circular dichroism), in line with thermostability changes associated with FR mutation alone rather than disulfide linkage formation (26), and were not investigated further. By contrast, introduction of putative non-canonical disulfide linkages formed between Cys24-Cys86, Cys4-Cys25 and Cys6-Cys119 resulted in average increases in Ig V domain Tₘ of 12-15 °C by circular dichroism, while introduction of the putative Cys22-Cys88 non-canonical disulfide linkage conferred an intermediate phenotype, increasing Ig V domain Tₘ by an average of 6.1 °C with high variability. All four putative disulfide linkages generally conferred larger thermostability enhancements on V_{H}s than V_{H}Hs or V_{L}s. Increased thermostability conferred by these four putative disulfide linkages was achieved at the cost of variable but generally modest decreases in the thermal refolding efficiency (α-value) and monomericity of V_{H}Hs, while the impacts on solubility and refolding of V_{H} and V_{L} domains were more variable and less clear. The binding affinities of most antigen-specific V_{H}Hs were unaffected or only slightly weakened (ΔK_{D}<5) by introduction of the four putative non-canonical disulfide linkages. Introduction of selected S-S linkages into the domains tested did not appear to abrogate protein A binding (for V_{H}Hs and V_{H}S) or protein L binding (for V_{L}s).

TABLE 4
Biophysical properties, thermal stability, and equilibrium dissociation
constants (K_{D}s) of wild-type sdAbs and sdAbs bearing putative
non-canonical disulfide linkages.

| sdAb | | Disulfide | M_{for} | M_{MALS} | Monomer | Tₘ | Fraction | K_{D} | pA | pL |
|---|---|---|---|---|---|---|---|---|---|---|
| type | sdAb | linkage(s) | (Da)^{a} | (Da)^{b} | (%)^{c} | (°C)^{d} | refolded^{d} | (nM)^{e} | binding^{e} | binding^{e} |
| | A4.2 | 23-104 | 15727 | 15023 | 98.0 | 87.2 ± 0.1 | 0.97 ± 0.01 | 5.0 | | n.a. |
| | | 23-104, 22-88 | 15683 | 16250 | 88.7 | 89.7 ± 0.2 | 0.48 ± 0.01 | 61.6 | | n.a. |
| | | 23-104, 24-86 | 15761 | 15675 | 96.9 | 97.4 ± 0.1 | 0.65 ± 0.01 | 92.0 | | n.a. |
| | | 23-104, 45-100 | 15791 | n.d. | n.d. | 91.8 ± 0.1 | 0.55 ± 0.02 | n.d. | | n.a. |
| | | 23-104, 4-25 | 15749 | n.d. | 96.2 | 93.7 ± 0.2 | 0.66 ± 0.01 | 8.7 | | n.a. |
| | | 23-104, 4-118 | 15674 | 15740 | 88.3 | 87.2 ± 0.1 | 0.64 ± 0.02 | 63.7 | | n.a. |
| | | 23-104, 5-120 | 15676 | n.d. | n.d. | 91.5 ± 0.1 | 0.77 ± 0.02 | n.d. | | n.a. |
| | | 23-104, 6-119 | 15747 | n.d. | 90.7 | 96.5 ± 0.2 | 0.36 ± 0.02 | 7.4 | | n.a. |
| | A5.1 | 23-104 | 15802 | 12903 | 99.8 | 77.1 ± 0.1 | 0.82 ± 0.01 | 5.1 | | n.a. |
| | | 23-104, 22-88 | 15758 | n.d. | 97.0 | 80.1 ± 0.2 | 0.60 ± 0.01 | 4.8 | | n.a. |
| | | 23-104, 24-86 | 15836 | 15495 | 97.7 | 90.0 ± 0.6 | 0.48 ± 0.03 | 5.2 | | n.a. |
| | | 23-104, 45-100 | 15867 | n.d. | n.d. | 79.6 ± 0.4 | 0.15 ± 0.01 | n.d. | | n.a. |
| | | 23-104, 4-25 | 15824 | n.d. | 96.4 | 86.6 ± 0.1 | 0.56 ± 0.01 | 2.6 | | n.a. |
| | | 23-104, 4-118 | 15709 | 15313 | 88.8 | 74.9 ± 0.3 | 0.60 ± 0.01 | 214 | | n.a. |
| | | 23-104, 5-120 | 15751 | n.d. | n.d. | 80.0 ± 0.4 | 0.42 ± 0.01 | n.d. | | n.a. |
| | | 23-104, 6-119 | 15823 | n.d. | 98.4 | 92.0 ± 0.2 | 0.62 ± 0.01 | 1.6 | | n.a. |
| | A20.1 | 23-104 | 15714 | 16810 | 96.2 | 73.5 ± 0.1 | 0.86 ± 0.02 | 1.7 | | n.a. |
| | | 23-104, 22-88 | 15626 | n.d. | n.d. | n.d. | n.d. | n.d. | | n.a. |
| | | 23-104, 24-86 | 15704 | 14865 | 98.9 | 88.2 ± 0.2 | 0.44 ± 0.05 | 1.7 | | n.a. |
| | | 23-104, 45-100 | 15752 | n.d. | n.d. | 77.5 ± 0.1 | 0.35 ± 0.03 | n.d. | | n.a. |
| | | 23-104, 4-25 | 15736 | n.d. | 98.6 | 82.5 ± 0.2 | 0.58 ± 0.02 | 1.5 | | n.a. |
| | | 23-104, 4-118 | 15577 | 14732 | 75.1 | 69.3 ± 0.4 | 0.78 ± 0.02 | n.d. | | n.a. |
| | | 23-104, 5-120 | 15693 | n.d. | n.d. | 74.9 ± 0.1 | 0.47 ± 0.01 | n.d. | | n.a. |
| | | 23-104, 6-119 | 15734 | 14480 | 85.3 | 89.1 ± 0.1 | 0.32 ± 0.01 | 6.3 | | n.a. |
| | A24.1 | 23-104 | 15767 | 16233 | 95.7 | 76.3 ± 0.1 | 0.85 ± 0.03 | 151 | | n.a. |
| | | 23-104, 22-88 | 15723 | 16030 | 81.8 | 79.0 ± 0.2 | 0.69 ± 0.01 | 647 | | n.a. |
| | | 23-104, 24-86 | 15801 | 15430 | 89.5 | 85.7 ± 0.1 | 0.48 ± 0.01 | 355 | | n.a. |
| | | 23-104, 45-100 | 15805 | n.d. | n.d. | 78.6 ± 0.1 | 0.35 ± 0.04 | n.d. | | n.a. |
| | | 23-104, 4-25 | 15789 | 14480 | 97.9 | 87.0 ± 0.1 | 0.31 ± 0.02 | 1060 | | n.a. |
| | | 23-104, 4-118 | 15674 | 15740 | 82.1 | 79.2 ± 0.1 | 0.73 ± 0.01 | 2140 | | n.a. |
| | | 23-104, 5-120 | 15746 | n.d. | n.d. | 89.4 ± 0.1 | 0.48 ± 0.01 | n.d. | | n.a. |
| | | 23-104, 6-119 | 15787 | n.d. | 58.8 | 76.2 ± 0.4 | 0.51 ± 0.02 | 413 | | n.a. |
| | A26.8 | 23-104 | 16016 | 16413 | 99.2 | 83.5 ± 0.3 | n.d. | 16.5 | | n.a. |
| | | 23-104, 22-88 | 15973 | 19270 | 75.1 | 86.4 ± 0.2 | 0.70 ± 0.01 | 18.7 | | n.a. |
| | | 23-104, 24-86 | 16051 | 16205 | 85.1 | 95.1 ± 0.2 | n.d. | 43.6 | | n.a. |
| | | 23-104, 45-100 | 16081 | n.d. | n.d. | 85.0 ± 0.1 | 0.65 ± 0.01 | n.d. | | n.a. |
| | | 23-104, 4-25 | 16039 | n.d. | 97.1 | 91.7 ± 0.1 | 0.71 ± 0.01 | 12.6 | | n.a. |
| | | 23-104, 4-118 | 15924 | 14725 | 97.3 | 92.7 ± 0.2 | 0.81 ± 0.01 | 43.4 | | n.a. |
| | | 23-104, 5-120 | 15966 | n.d. | n.d. | 91.1 | n.d. | n.d. | | n.a. |
| | | 23-104, 6-119 | 16037 | n.d. | n.d. | 101.1 ± 0.1 | n.d. | 12.6 | | n.a. |
| | B39 | 23-104 | 15608 | 12785 | 96.9 | 83.3 ± 0.7 | 0.76 ± 0.01 | 0.074 | | n.a. |
| | | 23-104, 22-88 | 15664 | n.d. | 97.0 | 91.1 ± 0.1 | 0.71 ± 0.01 | n.d. | | n.a. |
| | | 23-104, 24-86 | 15642 | 12950 | 96.5 | 97.5 | 0.77 | n.d. | | n.a. |
| | | 23-104, 45-100 | 15672 | n.d. | n.d. | 84.5 ± 0.1 | 0.16 ± 0.05 | n.d. | | n.a. |
| | | 23-104, 4-25 | 15630 | n.d. | n.d. | 91.3 ± 0.1 | 0.70 ± 0.01 | 0.023 | | n.a. |
| | | 23-104, 4-118 | 15515 | 15470 | 98.5 | 80.2 ± 0.6 | 0.63 ± 0.01 | n.d. | | n.a. |
| | | 23-104, 5-120 | 15658 | n.d. | n.d. | 82.0 ± 0.1 | 0.44 ± 0.02 | n.d. | | n.a. |
| | | 23-104, 6-119 | 15628 | n.d. | n.d. | 92.8 ± 1.0 | 0.57 ± 0.02 | 0.028 | | n.a. |
| | IGF1R3 | 23-104 | 16215 | 15275 | 99.4 | 76.7 ± 0.6 | n.d. | 0.9 | | n.a. |
| | | 23-104, 22-88 | 16219 | 19140 | 96.1 | 85.5 ± 0.6 | 0.63 ± 0.01 | 0.8 | | n.a. |
| | | 23-104, 24-86 | 16221 | 15373 | 98.1 | 88.5 ± 0.1 | 0.67 ± 0.01 | 5.3 | | n.a. |
| | | 23-104, 45-100 | 16279 | n.d. | n.d. | 76.0 ± 0.2 | n.d. | n.d. | | n.a. |
| | | 23-104, 4-25 | 16237 | n.d. | 94.6 | 85.5 ± 0.1 | n.d. | 2.3 | | n.a. |
| | | 23-104, 4-118 | 16122 | 15060 | 95.3 | 79.9 | 0.52 | n.d. | | n.a. |
| | | 23-104, 5-120 | 16164 | n.d. | n.d. | 76.4 ± 0.1 | 0.42 ± 0.01 | n.d. | | n.a. |
| | | 23-104, 6-119 | 16235 | n.d. | 76.0 | 88.9 ± 0.5 | 0.43 ± 0.01 | 1.4 | | n.a. |
| | IGF1R4 | 23-104 | 15733 | 14530 | 99.2 | 80.1 ± 0.1 | 0.79 ± 0.01 | 0.3 | | n.a. |
| | | 23-104, 22-88 | 15689 | 14338 | 98.0 | 85.1 ± 0.8 | 0.70 ± 0.02 | 0.5 | | n.a. |
| | | 23-104, 24-86 | 15709 | 15795 | 97.6 | 95.6 ± 0.1 | 0.69 ± 0.01 | 1.1 | | n.a. |
| | | 23-104, 45-100 | 15798 | n.d. | n.d. | 83.4 ± 0.1 | 0.44 ± 0.01 | n.d. | | n.a. |
| | | 23-104, 4-25 | 15727 | n.d. | 94.6 | 87.4 ± 0.1 | 0.39 ± 0.01 | 0.5 | | n.a. |
| | | 23-104, 4-118 | 15640 | 16548 | 75.6 | n.d. | n.d. | n.d. | | n.a. |
| | | 23-104, 5-120 | 15682 | n.d. | n.d. | 82.3 ± 0.1 | 0.51 ± 0.03 | n.d. | | n.a. |
| | | 23-104, 6-119 | 15754 | n.d. | 97.9 | 91.7 ± 0.4 | 0.73 ± 0.01 | 0.3 | | n.a. |
| | IGF1R5 | 23-104 | 15747 | 13603 | 99.6 | 61.9 ± 0.7 | 0.64 ± 0.02 | 1.1 | | n.a. |
| | | 23-104, 22-88 | 15703 | 14360 | 99.1 | 67.1 ± 0.1 | 0.38 ± 0.01 | 1.4 | | n.a. |
| | | 23-104, 24-86 | 15781 | n.d. | n.d. | n.d. | n.d. | n.d. | | n.a. |
| | | 23-104, 45-100 | 15811 | n.d. | n.d. | 65.8 ± 0.2 | 0.46 ± 0.01 | n.d. | | n.a. |
| | | 23-104, 4-25 | 15769 | n.d. | 98.8 | 73.3 ± 0.1 | 0.48 ± 0.02 | 0.8 | | n.a. |
| | | 23-104, 4-118 | 15654 | 14025 | 99.3 | 66.3 ± 0.1 | 0.47 ± 0.01 | 4.7 | | n.a. |
| | | 23-104, 5-120 | 15696 | n.d. | n.d. | 69.4 ± 0.1 | 0.41 ± 0.01 | n.d. | | n.a. |
| | | 23-104, 6-119 | 15767 | n.d. | 98.2 | 78.5 ± 1.0 | 0.41 ± 0.01 | 0.5 | | n.a. |
| | EG2 | 23-104 | 16097 | 15545 | 94.9 | 78.9 ± 0.9 | 0.89 ± 0.03 | 14.6 | | n.a. |
| | | 23-104, 22-88 | 16053 | 14695 | 99.0 | 89.5 ± 0.7 | 0.72 ± 0.05 | 47.2 | | n.a. |
| | | 23-104, 24-86 | 16101 | 14073 | 95.2 | 91.6 ± 0.5 | 0.54 ± 0.04 | 62.5 | | n.a. |
| | | 23-104, 45-100 | 16161 | n.d. | n.d. | 82.4 ± 0.2 | 0.50 ± 0.02 | n.d. | | n.a. |
| | | 23-104, 4-25 | 16119 | 14335 | 90.4 | 88.4 ± 0.1 | 0.34 ± 0.01 | 10.6 | | n.a. |
| | | 23-104, 4-118 | 16004 | 15797 | 88.1 | 83.8 ± 0.7 | 0.67 ± 0.07 | n.d. | | n.a. |
| | | 23-104, 5-120 | 16046 | n.d. | n.d. | 80.1 ± 0.3 | 0.49 ± 0.01\| | n.d. | | n.a. |
| | | 23-104, 6-119 | 16117 | n.d. | 98.2 | 82.7 ± 0.5 | 0.69 ± 0.02 | 25.7 | | n.a. |
| | FC5 | 23-104 | 15514 | 16990 | 95.6 | 66.1 ± 0.6 | 0.60 ± 0.01 | n.d. | Y | n.a. |
| | | | | | | | | | (1.4 μM) | |
| | | 23-104, 22-88 | 15470 | 16720 | 99.0 | 70.1 ± 0.6 | 0.58 ± 0.02 | n.d. | | n.a. |
| | | 23-104, 24-86 | 15548 | 13540 | 95.7 | 81.6 ± 0.4 | n.d. | n.d. | | n.a. |
| | | 23-104, 45-100 | 15578 | n.d. | n.d. | 69.6 ± 0.1 | 0.30 ± 0.02 | n.d. | | n.a. |
| | | 23-104, 4-25 | 15536 | n.d. | 97.8 | 77.8 ± 0.1 | 0.51 ± 0.01 | n.d. | | n.a. |
| | | 23-104, 4-118 | 15421 | n.d. | n.d. | n.d. | n.d. | n.d. | | n.a. |
| | | 23-104, 5-120 | 15464 | n.d. | n.d. | 73.6 ± 0.1 | 0.62 ± 0.01 | n.d. | | n.a. |
| | | 23-104, 6-119 | 15592 | n.d. | 98.4 | 77.4 ± 0.1 | 0.51 ± 0.01 | n.d. | | n.a. |
| V_{H} | VH413 | 23-104 | 15040 | n.d. | n.d. | 54.2 | n.d. | n.d. | | n.a. |
| | | 23-104, 22-88 | 15133 | n.d. | n.d. | 72.7 | n.d. | n.d. | Y | n.a. |
| | | 23-104, 24-86 | 15074 | n.d. | n.d. | 79.1 | n.d. | n.d. | Y | n.a. |
| | | 23-104, 45-100 | 15241 | n.d. | n.d. | n.d. | n.d. | n.d. | | n.a. |
| | | 23-104, 4-25 | 15062 | n.d. | 80.7 | 73.5 ± 0.2 | n.d. | n.d. | Y | n.a. |
| | | 23-104, 4-118 | 15084 | n.d. | n.d. | n.d. | n.d. | n.d. | Y | n.a. |
| | | 23-104, 5-120 | 15156 | n.d. | n.d. | n.d. | n.d. | n.d. | | n.a. |
| | | 23-104, 6-119 | 15198 | n.d. | 90.1 | 86.2 ± 0.3 | n.d. | n.d. | Y | n.a. |
| | VH414 | 23-104 | 14968 | n.d. | 79.5 | 58.7 ± 0.1 | n.d. | n.d. | | n.a. |
| | | 23-104, 22-88 | 15061 | n.d. | n.d. | n.d. | n.d. | n.d. | | n.a. |
| | | 23-104, 24-86 | 14972 | 12945 | 97.4 | 69.6 ± 0.1 | n.d. | n.d. | Y | n.a. |
| | | | | | | | | | (2.2 μM) | |
| | | 23-104, 45-100 | 15169 | n.d. | n.d. | n.d. | n.d. | n.d. | | n.a. |
| | | 23-104, 4-25 | 15127 | n.d. | 48.7 | 72.9 ± 0.1 | n.d. | n.d. | Y | n.a. |
| | | 23-104, 4-118 | 15012 | n.d. | n.d. | n.d. | n.d. | n.d. | | n.a. |
| | | 23-104, 5-120 | 15084 | n.d. | n.d. | n.d. | n.d. | n.d. | | n.a. |
| | | 23-104, 6-119 | 15126 | n.d. | 79.2 | 79.1 ± 0.6 | n.d. | n.d. | Y | n.a. |
| | VH419 | 23-104 | 14947 | n.d. | n.d. | 58.9 ± 0.2 | 0.35 ± 0.03 | n.d. | | n.a. |
| | | 23-104, 22-88 | 15040 | n.d. | n.d. | n.d. | n.d. | n.d. | | n.a. |
| | | 23-104, 24-86 | 14995 | 13515 | 98.6 | 70.8 ± 0.1 | 0.30 ± 0.05 | n.d. | Y | n.a. |
| | | | | | | | | | (3.7 μM) | |
| | | 23-104, 45-100 | 15149 | n.d. | n.d. | n.d. | n.d. | n.d. | | n.a. |
| | | 23-104, 4-25 | 15106 | n.d. | 93.3 | 73.2 ± 0.3 | n.d. | n.d. | Y | n.a. |
| | | 23-104, 4-118 | 14991 | n.d. | n.d. | n.d. | n.d. | n.d. | | n.a. |
| | | 23-104, 5-120 | 15034 | n.d. | n.d. | n.d. | n.d. | n.d. | | n.a. |
| | | 23-104, 6-119 | 15105 | 13180 | 84.8 | 73.5 ± 0.6 | n.d. | n.d. | Y | n.a. |
| | VH420 | 23-104 | 14932 | n.d. | 78.5 | 58.1 ± 0.1 | n.d. | n.d. | Y | n.a. |
| | | | | | | | | | (2.6 μM) | |
| | | 23-104, 22-88 | 14881 | n.d. | n.d. | n.d. | n.d. | n.d. | | n.a. |
| | | 23-104, 24-86 | 14966 | 12820 | 96.3 | 69.3 ± 0.2 | n.d. | n.d. | Y | n.a. |
| | | | | | | | | | (1.4 μM) | |
| | | 23-104, 45-100 | 15133 | n.d. | n.d. | n.d. | n.d. | n.d. | | n.a. |
| | | 23-104, 4-25 | 15091 | n.d. | 86.0 | 73.8 ± 0.1 | n.d. | n.d. | Y | n.a. |
| | | 23-104, 4-118 | 14976 | n.d. | n.d. | n.d. | n.d. | n.d. | | n.a. |
| | | 23-104, 5-120 | 15048 | n.d. | n.d. | n.d. | n.d. | n.d. | | n.a. |
| | | 23-104, 6-119 | 15089 | 15665 | 70.9 | 77.4 ± 0.2 | n.d. | n.d. | Y | n.a. |
| | VH421 | 23-104 | 15392 | n.d. | 83.9 | 58.8 ± 0.1 | n.d. | n.d. | | n.a. |
| | | 23-104, 22-88 | 15348 | n.d. | 93.6 | n.d. | n.d. | n.d. | Y | n.a. |
| | | 23-104, 24-86 | 15289 | 15580 | 94.6 | 69.4 ± 0.1 | n.d. | n.d. | Y | n.a. |
| | | | | | | | | | (1.2 μM) | |
| | | 23-104, 45-100 | 15456 | n.d. | n.d. | n.d. | n.d. | n.d. | | n.a. |
| | | 23-104, 4-25 | 15414 | n.d. | 85.7 | 74.5 ± 0.4 | n.d. | n.d. | Y | n.a. |
| | | 23-104, 4-118 | 15299 | n.d. | n.d. | n.d. | n.d. | n.d. | | n.a. |
| | | 23-104, 5-120 | 15342 | n.d. | n.d. | n.d. | n.d. | n.d. | | n.a. |
| | | 23-104, 6-119 | 15412 | n.d. | 76.0 | 78.3 | n.d. | n.d. | Y | n.a. |
| | VH423 | 23-104 | 14966 | n.d. | n.d. | 57.0 ± 1.0 | n.d. | n.d. | | n.a. |
| | | 23-104, 22-88 | 15059 | n.d. | n.d. | n.d. | n.d. | n.d. | | n.a. |
| | | 23-104, 24-86 | 15000 | 14075 | 94.3 | 76.0 ± 0.1 | n.d. | n.d. | Y | n.a. |
| | | | | | | | | | (2.9 μM) | |
| | | 23-104, 45-100 | 15167 | n.d. | n.d. | n.d. | n.d. | n.d. | | n.a. |
| | | 23-104, 4-25 | 15125 | n.d. | 60.4 | 66.5 ± 0.7 | n.d. | n.d. | Y | n.a. |
| | | 23-104, 4-118 | 15010 | n.d. | n.d. | n.d. | n.d. | n.d. | | n.a. |
| | | 23-104, 5-120 | 15082 | n.d. | n.d. | n.d. | n.d. | n.d. | | n.a. |
| | | 23-104, 6-119 | 15124 | n.d. | 93.5 | 82.2 ± 0.1 | n.d. | n.d. | Y | n.a. |
| | VH428 | 23-104 | 15675 | n.d. | 94.4 | 62.3 | n.d. | n.d. | Y | n.a. |
| | | | | | | | | | (3.8 μM) | |
| | | 23-104, 22-88 | 15631 | n.d. | 86.8 | 64.6 | n.d. | n.d. | Y | n.a. |
| | | 23-104, 24-86 | 15560 | n.d. | n.d. | 77.4 | n.d. | n.d. | Y | n.a. |
| | | 23-104, 45-100 | 15739 | n.d. | n.d. | n.d. | n.d. | n.d. | Y | n.a. |
| | | 23-104, 4-25 | 15697 | n.d. | 67.7 | 76.5 ± 0.2 | n.d. | n.d. | Y | n.a. |
| | | 23-104, 4-118 | 15582 | n.d. | n.d. | n.d. | n.d. | n.d. | Y | n.a. |
| | | 23-104, 5-120 | 15625 | n.d. | n.d. | n.d. | n.d. | n.d. | | n.a. |
| | | 23-104, 6-119 | 15695 | 15430 | 84.7 | 83.2 ± 0.2 | n.d. | n.d. | Y | n.a. |
| | VH429 | 23-104 | 14959 | n.d. | 88.0 | 59.9 ± 0.1 | 0.48 ± 0.01 | n.d. | Y | n.a. |
| | | | | | | | | | (1.9 μM) | |
| | | 23-104, 22-88 | 15053 | n.d. | n.d. | n.d. | n.d. | n.d. | | n.a. |
| | | 23-104, 24-86 | 14994 | 13570 | 96.1 | 65.2 ± 0.1 | 0.26 ± 0.01 | n.d. | Y | n.a. |
| | | | | | | | | | (1.3 μM) | |
| | | 23-104, 45-100 | 15161 | n.d. | n.d. | n.d. | n.d. | n.d. | | n.a. |
| | | 23-104, 4-25 | 15119 | n.d. | 83.7 | 70.1 ± 0.1 | n.d. | n.d. | Y | n.a. |
| | | 23-104, 4-118 | 15004 | n.d. | n.d. | n.d. | n.d. | n.d. | | n.a. |
| | | 23-104, 5-120 | 15076 | n.d. | n.d. | n.d. | n.d. | n.d. | | n.a. |
| | | 23-104, 6-119 | 15117 | n.d. | n.d. | n.d. | n.d. | n.d. | n.d. | n.a. |
| | VHM41 | 23-104 | 15333 | n.d. | 47.5 | 52.8 ± 2.2 | n.d. | n.d. | | n.a. |
| | | 23-104, 22-88 | 15426 | n.d. | n.d. | n.d. | n.d. | n.d. | | n.a. |
| | | 23-104, 24-86 | 15367 | 15220 | 76.4 | 75.0 ± 0.3 | n.d. | n.d. | Y | n.a. |
| | | | | | | | | | (0.5 μM) | |
| | | 23-104, 45-100 | 15534 | n.d. | n.d. | n.d. | n.d. | n.d. | | n.a. |
| | | 23-104, 4-25 | 15492 | n.d. | 29.8 | 77.4 ± 0.1 | n.d. | n.d. | Y | n.a. |
| | | 23-104, 4-118 | 15377 | n.d. | n.d. | n.d. | n.d. | n.d. | | n.a. |
| | | 23-104, 5-120 | 15449 | n.d. | n.d. | n.d. | n.d. | n.d. | | n.a. |
| | | 23-104, 6-119 | 15491 | n.d. | 70.0 | 79.5 ± 0.1 | n.d. | n.d. | Y | n.a. |
| | VH44 | 23-104 | 15274 | n.d. | 92.8 | 67.9 ± 0.1 | 0.51 ± 0.01 | n.d. | Y | n.a. |
| | | | | | | | | | (1.9 μM) | |
| | | 23-104, 22-88 | 15367 | n.d. | 72.1 | n.d. | n.d. | n.d. | Y | n.a. |
| | | 23-104, 24-86 | 15308 | 13910 | 94.1 | 76.3 ± 0.2 | 0.40 ± 0.02 | n.d. | Y | n.a. |
| | | | | | | | | | (1.6 μM) | |
| | | 23-104, 45-100 | 15475 | n.d. | n.d. | n.d. | n.d. | n.d. | | n.a. |
| | | 23-104, 4-25 | 15433 | n.d. | 75.7 | 80.9 ± 0.1 | n.d. | n.d. | Y | n.a. |
| | | 23-104, 4-118 | 15318 | n.d. | n.d. | n.d. | n.d. | n.d. | | n.a. |
| | | 23-104, 5-120 | 15361 | n.d. | n.d. | n.d. | n.d. | n.d. | | n.a. |
| | | 23-104, 6-119 | 15431 | n.d. | 63.5 | 79.7 ± 0.1 | n.d. | n.d. | Y | n.a. |
| | VHM81 | 23-104 | 15058 | n.d. | 94.7 | 69.6 ± 0.4 | n.d. | n.d. | Y | n.a. |
| | | | | | | | | | (2.3 μM) | |
| | | 23-104, 22-88 | 15151 | 16910 | 85.2 | n.d. | n.d. | n.d. | Y | n.a. |
| | | 23-104, 24-86 | 15092 | 15330 | 97.2 | 80.2 ± 0.1 | n.d. | n.d. | Y | n.a. |
| | | | | | | | | | (1.0 μM) | |
| | | 23-104, 45-100 | 15259 | n.d. | n.d. | n.d. | n.d. | n.d. | | n.a. |
| | | 23-104, 4-25 | 15217 | n.d. | 77.4 | 81.0 ± 0.1 | n.d. | n.d. | Y | n.a. |
| | | 23-104, 4-118 | 15102 | n.d. | n.d. | n.d. | n.d. | n.d. | | n.a. |
| | | 23-104, 5-120 | 15174 | n.d. | n.d. | n.d. | n.d. | n.d. | | n.a. |
| | | 23-104, 6-119 | 15216 | n.d. | 78.7 | 84.4 ± 0.1 | n.d. | n.d. | Y | n.a. |
| | VHB82 | 23-104 | 14141 | n.d. | 53.2 | 60.7 ± 0.1 | 0.37 ± 0.01 | n.d. | Y | n.a. |
| | | | | | | | | | (2.1 uM) | |
| | | 23-104, 22-88 | 14234 | n.d. | n.d. | n.d. | n.d. | n.d. | | n.a. |
| | | 23-104, 24-86 | 14175 | 14190 | 82.7 | 75.6 ± 0.1 | 0.36 ± 0.01 | n.d. | Y | n.a. |
| | | | | | | | | | (1.5 μM) | |
| | | 23-104, 45-100 | 14342 | n.d. | n.d. | n.d. | n.d. | n.d. | | n.a. |
| | | 23-104, 4-25 | 14230 | 12375 | 89.4 | 83.5 ± 0.1 | n.d. | n.d. | Y | n.a. |
| | | 23-104, 4-118 | 14185 | n.d. | n.d. | n.d. | n.d. | n.d. | | n.a. |
| | | 23-104, 5-120 | 14228 | n.d. | n.d. | n.d. | n.d. | n.d. | | n.a. |
| | | 23-104, 6-119 | 14298 | n.d. | 97.0 | 75.7 ± 0.2 | n.d. | n.d. | Y | n.a. |
| V_{L} | VL324 | 23-104 | 13832 | 13180 | 68.6 | 66.1 | n.d. | n.d. | n.a. | |
| | | 23-104, 22-88 | 13836 | n.d. | 100.0 | n.d. | n.d. | n.d. | n.a. | Y |
| | | 23-104, 24-86 | 13767 | 12510 | 98.0 | n.d. | n.d. | n.d. | n.a. | n.d. |
| | | 23-104, 45-100 | 13839 | n.d. | n.d. | n.d. | n.d. | n.d. | n.a. | |
| | | 23-104, 4-25 | 13836 | n.d. | 93.1 | 81.7 ± 0.4 | n.d. | n.d. | n.a. | Y |
| | | 23-104, 4-118 | 13760 | n.d. | n.d. | n.d. | n.d. | n.d. | n.a. | |
| | | 23-104, 5-120 | 13800 | n.d. | n.d. | n.d. | n.d. | n.d. | n.a. | |
| | | 23-104, 6-119 | 13853 | n.d. | 79.9 | 81.1 | n.d. | n.d. | n.a. | Y |
| | VL325 | 23-104 | 13608 | 13205 | 80.9 | 70.8 | 0.86 ± 0.01 | n.d. | n.a. | |
| | | 23-104, 22-88 | 13626 | n.d. | n.d. | n.d. | n.d. | n.d. | n.a. | n.d. |
| | | 23-104, 24-86 | 13545 | 14230 | 100.0 | 76.8 ± 0.1 | 0.79 ± 0.01 | n.d. | n.a. | Y |
| | | 23-104, 45-100 | 13587 | n.d. | n.d. | n.d. | n.d. | n.d. | n.a. | |
| | | 23-104, 4-25 | 13630 | 13475 | 97.2 | 79.4 ± 0.6 | n.d. | n.d. | n.a. | Y |
| | | 23-104, 4-118 | 13554 | n.d. | n.d. | n.d. | n.d. | n.d. | n.a. | |
| | | 23-104, 5-120 | 13656 | n.d. | n.d. | n.d. | n.d. | n.d. | n.a. | |
| | | 23-104, 6-119 | 13629 | n.d. | n.d. | n.d. | n.d. | n.d. | n.a. | n.d. |
| | VL330 | 23-104 | 13572 | 13825 | 81.6 | 63.8 | 0.85 ± 0.01 | n.d. | n.a. | |
| | | 23-104, 22-88 | 13576 | n.d. | n.d. | n.d. | n.d. | n.d. | n.a. | n.d. |
| | | 23-104, 24-86 | 13509 | 10525 | 93.2 | 66.0 ± 0.1 | 0.90 ± 0.01 | n.d. | n.a. | Y |
| | | 23-104, 45-100 | 13549 | n.d. | n.d. | n.d. | n.d. | n.d. | n.a. | |
| | | 23-104, 4-25 | 13576 | n.d. | 87.8 | 80.6 ± 1.1 | n.d. | n.d. | n.a. | Y |
| | | 23-104, 4-118 | 13500 | n.d. | n.d. | n.d. | n.d. | n.d. | n.a. | |
| | | 23-104, 5-120 | 13540 | n.d. | n.d. | n.d. | n.d. | n.d. | n.a. | |
| | | 23-104, 6-119 | 13593 | n.d. | 91.5 | 79.4 ± 0.1 | n.d. | n.d. | n.a. | Y |
| | VL335 | 23-104 | 13904 | n.d. | n.d. | 63.5 | 0.88 ± 0.01 | n.d. | n.a. | |
| | | 23-104, 22-88 | 13922 | n.d. | 97.5 | 71.0 ± 1.2 | n.d. | n.d. | n.a. | Y |
| | | 23-104, 24-86 | 13817 | n.d. | n.d. | 69.8 ± 0.2 | 0.70 ± 0.02 | n.d. | n.a. | Y |
| | | 23-104, 45-100 | 13911 | n.d. | n.d. | n.d. | n.d. | n.d. | n.a. | |
| | | 23-104, 4-25 | 13908 | n.d. | 94.6 | 78.8 ± 1.0 | n.d. | n.d. | n.a. | Y |
| | | 23-104, 4-118 | 13832 | n.d. | n.d. | n.d. | n.d. | n.d. | n.a. | |
| | | 23-104, 5-120 | 13881 | n.d. | n.d. | n.d. | n.d. | n.d. | n.a. | |
| | | 23-104, 6-119 | 13925 | n.d. | 76.1 | 76.6 ± 0.4 | n.d. | n.d. | n.a. | Y |
| | VL342 | 23-104 | 13950 | n.d. | n.d. | 58.4 | n.d. | n.d. | n.a. | |
| | | 23-104, 22-88 | 13899 | n.d. | 76.3 | 61.0 ± 0.8 | n.d. | n.d. | n.a. | n.d. |
| | | 23-104, 24-86 | 13871 | n.d. | n.d. | n.d. | n.d. | n.d. | n.a. | n.d. |
| | | 23-104, 45-100 | 13929 | n.d. | n.d. | n.d. | n.d. | n.d. | n.a. | |
| | | 23-104, 4-25 | 13938 | n.d. | 94.1 | 78.9 ± 0.3 | n.d. | n.d. | n.a. | Y |
| | | 23-104, 4-118 | 13878 | n.d. | n.d. | n.d. | n.d. | n.d. | n.a. | |
| | | 23-104, 5-120 | 13942 | n.d. | n.d. | n.d. | n.d. | n.d. | n.a. | |
| | | 23-104, 6-119 | 13972 | n.d. | n.d. | n.d. | n.d. | n.d. | n.a. | n.d. |
| | VL351 | 23-104 | 13854 | 14800 | 91.8 | 62.0 | n.d. | n.d. | n.a. | |
| | | 23-104, 22-88 | 13872 | n.d. | 76.2 | 64.5 ± 0.7 | n.d. | n.d. | n.a. | Y |
| | | 23-104, 24-86 | 13789 | 12810 | 977 | 68.9 ± 0.2 | n.d. | n.d. | n.a. | |
| | | 23-104, 45-100 | 13861 | n.d. | n.d. | n.d. | n.d. | n.d. | n.a. | |
| | | 23-104, 4-25 | 13858 | n.d. | 93.8 | 73.6 ± 0.4 | n.d. | n.d. | n.a. | Y |
| | | 23-104, 4-118 | 13782 | n.d. | n.d. | n.d. | n.d. | n.d. | n.a. | |
| | | 23-104, 5-120 | 13902 | n.d. | n.d. | n.d. | n.d. | n.d. | n.a. | |
| | | 23-104, 6-119 | 13875 | n.d. | 77.4 | 74.5 ± 0.1 | n.d. | n.d. | n.a. | Y |
| | VL364 | 23-104 | 13945 | 13685 | 86.0 | 58.7 | 0.84 ± 0.01 | n.d. | n.a. | |
| | | 23-104, 22-88 | 13963 | 14360 | 81.2 | 66.1 ± 0.4 | n.d. | n.d. | n.a. | Y |
| | | 23-104, 24-86 | 13880 | 13220 | 98.5 | 65.5 ± 0.1 | 0.68 ± 0.01 | n.d. | n.a. | Y |
| | | 23-104, 45-100 | 13952 | n.d. | n.d. | n.d. | n.d. | n.d. | n.a. | |
| | | 23-104, 4-25 | 13967 | n.d. | 91.8 | 70.8 ± 0.2 | n.d. | n.d. | n.a. | Y |
| | | 23-104, 4-118 | 13891 | n.d. | n.d. | n.d. | n.d. | n.d. | n.a. | |
| | | 23-104, 5-120 | 13922 | n.d. | n.d. | n.d. | n.d. | n.d. | n.a. | |
| | | 23-104, 6-119 | 13993 | n.d. | 98.7 | 74.3 ± 0.1 | n.d. | n.d. | n.a. | Y |
| | VL382 | 23-104 | 13931 | 13905 | 84.3 | 72.6 | 0.92 ± 0.01 | n.d. | n.a. | |
| | | 23-104, 22-88 | 13950 | 14820 | 89.3 | 74.6 ± 0.1 | n.d. | n.d. | n.a. | Y |
| | | 23-104, 24-86 | 13866 | 12520 | 96.6 | 79.4 ± 2.1 | 0.77 ± 0.01 | n.d. | n.a. | Y |
| | | 23-104, 45-100 | 13938 | n.d. | n.d. | n.d. | n.d. | n.d. | n.a. | |
| | | 23-104, 4-25 | 13935 | n.d. | 60.8 | 59.1 ± 0.2 | n.d. | n.d. | n.a. | Y |
| | | 23-104, 4-118 | 13859 | n.d. | n.d. | n.d. | n.d. | n.d. | n.a. | |
| | | 23-104, 5-120 | 13908 | n.d. | n.d. | n.d. | n.d. | n.d. | n.a. | |
| | | 23-104, 6-119 | 13953 | n.d. | 90.6 | 85.4 ± 0.3 | n.d. | n.d. | n.a. | Y |
| | VL383 | 23-104 | 13796 | 14665 | 76.8 | 58.9 | 0.89 ± 0.01 | n.d. | n.a. | |
| | | 23-104, 22-88 | 13814 | n.d. | 94.0 | 65.9 ± 0.1 | n.d. | n.d. | n.a. | Y |
| | | 23-104, 24-86 | 13866 | 12285 | 94.1 | 67.8 ± 0.1 | 0.65 ± 0.01 | n.d. | n.a. | Y |
| | | 23-104, 45-100 | 13803 | n.d. | n.d. | n.d. | n.d. | n.d. | n.a. | |
| | | 23-104, 4-25 | 13818 | n.d. | 90.0 | 72.4 ± 0.1 | n.d. | n.d. | n.a. | Y |
| | | 23-104, 4-118 | 13742 | n.d. | n.d. | n.d. | n.d. | n.d. | n.a. | |
| | | 23-104, 5-120 | 13773 | n.d. | n.d. | n.d. | n.d. | n.d. | n.a. | |
| | | 23-104, 6-119 | 13817 | n.d. | 92.2 | 74.6 ± 0.1 | n.d. | n.d. | n.a. | Y |
| | VL389 | 23-104 | 13756 | 14040 | 89.6 | 54.5 | 0.77 ± 0.01 | n.d. | n.a. | |
| | | 23-104, 22-88 | 13774 | n.d. | 99.8 | 67.6 | n.d. | n.d. | n.a. | Y |
| | | 23-104, 24-86 | 13719 | 14095 | 100.0 | 70.6 ± 1.1 | 0.83 ± 0.01 | n.d. | n.a. | Y |
| | | 23-104, 45-100 | 13778 | n.d. | n.d. | n.d. | n.d. | n.d. | n.a. | |
| | | 23-104, 4-25 | 13806 | 13815 | 99.0 | 66.2 ± 0.1 | n.d. | n.d. | n.a. | Y |
| | | 23-104, 4-118 | 13716 | n.d. | n.d. | n.d. | n.d. | n.d. | n.a. | |
| | | 23-104, 5-120 | 13804 | n.d. | n.d. | n.d. | n.d. | n.d. | n.a. | |
| | | 23-104, 6-119 | 13777 | n.d. | 96.6 | 67.6 ± 0.1 | n.d. | n.d. | n.a. | Y |
| | VL3103 | 23-104 | 13852 | 14030 | 89.4 | 68.7 | 0.90 ± 0.01 | n.d. | n.a. | |
| | | 23-104, 22-88 | 13870 | n.d. | 83.2 | 72.5 | n.d. | n.d. | n.a. | Y |
| | | 23-104, 24-86 | 13787 | 12455 | 97.4 | 74.1 ± 0.2 | 0.67 ± 0.01 | n.d. | n.a. | Y |
| | | 23-104, 45-100 | 13831 | n.d. | n.d. | n.d. | n.d. | n.d. | n.a. | |
| | | 23-104, 4-25 | 13874 | 9980 | 59.0 | 76.5 ± 0.2 | n.d. | n.d. | n.a. | Y |
| | | 23-104, 4-118 | 13798 | n.d. | n.d. | n.d. | n.d. | n.d. | n.a. | |
| | | 23-104, 5-120 | 13829 | n.d. | n.d. | n.d. | n.d. | n.d. | n.a. | |
| | | 23-104, 6-119 | 13873 | n.d. | 98.7 | 83.7 ± 0.3 | n.d. | n.d. | n.a. | Y |

n.d., not determined;
n.a., not applicable;
pA, protein A;
pL, protein L
^{a}Calculated from the sdAb primary amino acid sequence including c-Myc and His6 tags
^{b}Calculated for the monomeric sdAb peak area from SEC-MALS data
^{c}Calculated from SEC-MALS data
^{d}Mean ± S.E.M. of duplicate circular dichroism measurements except for samples with limiting amounts
^{e}Measured by surface plasmon resonance

### Effects of Novel Disulfide Linkages on Ig V Domain Protease Resistance.

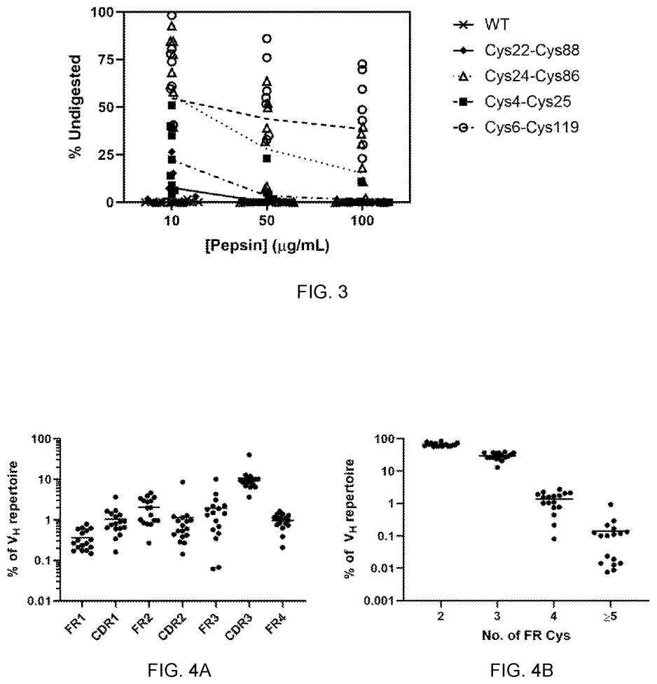

It was previously found that introduction of a non-canonical disulfide linkage formed between Cys54-Cys78 increased the pepsin resistance of V_{H}Hs (27) and V_{L}s (22). To assess whether any of the putative non-canonical disulfide linkages identified in this study had similar effects, the pepsin, trypsin, and chymotrypsin resistance of WT V_{H}Hs and engineered variants bearing non-canonical disulfide linkages formed between Cys22-Cys88, Cys24-Cys86, Cys4-Cys25, and Cys6-Cys119 was tested. As observed previously for the Cys54-Cys78 linkage, the putative Cys24-Cys86 and Cys6-Cys119 non-canonical disulfide linkages significantly increased the pepsin resistance of the majority of V_{H}Hs tested (FIG. 3 and Table 5). By contrast, the putative Cys22-Cys88 and Cys4-Cys25 linkages only modestly increased resistance to low concentrations of pepsin. None of the four putative non-canonical disulfide linkages had any effect on trypsin and chymotrypsin resistance (data not shown).

TABLE 5
Percent V_{H}H protein undigested following
1 h digestion with pepsin at 37 °C.

| sdAb | | Disulfide | Pepsin 10 | Pepsin 50 | Pepsin 100 |
|---|---|---|---|---|---|
| type | sdAb | linkage(s) | μg/mL (%) | μg/mL (%) | μg/mL (%) |
| V_{H}H | A4.2 | 23-104 | 0 | 0 | 0 |
| | | 23-104, 22-88 | 15 ± 4 | 0 | 0 |
| | | 23-104, 24-86 | 93 ± 3 | 64 ± 5 | 36 ± 7 |
| | | 23-104, 4-25 | 51 ± 13 | 1 ± 1 | 0 |
| | | 23-104, 6-119 | 81 ± 3 | 76 ± 2 | 70 ± 9 |
| | A5.1 | 23-104 | 0 | 0 | 0 |
| | | 23-104, 22-88 | 3 ± 2 | 0 | 0 |
| | | 23-104, 24-86 | 78 ± 2 | 32 ± 5 | 11 ± 3 |
| | | 23-104, 4-25 | 0 | 0 | 0 |
| | | 23-104, 6-119 | 73 ± 2 | 59 ± 6 | 60 ± 5 |
| | A20.1 | 23-104 | 0 | 0 | 0 |
| | | 23-104, 22-88 | n.d. | n.d. | n.d. |
| | | 23-104, 24-86 | 39 ± 2 | 7 ± 5 | 3 ± 2 |
| | | 23-104, 4-25 | 6 ± 6 | 0 | 0 |
| | | 23-104, 6-119 | 60 ± 2 | 35 ± 3 | 30 ± 3 |
| | A24.1 | 23-104 | 0 | 0 | 0 |
| | | 23-104, 22-88 | n.d. | n.d. | n.d. |
| | | 23-104, 24-86 | n.d. | n.d. | n.d. |
| | | 23-104, 4-25 | 40 ± 20 | 22 ± 8 | 11 ± 6 |
| | | 23-104, 6-119 | 98 ± 12 | 86 ± 10 | 73 ± 9 |
| | A26.8 | 23-104 | 0 | 0 | 0 |
| | | 23-104, 22-88 | 7 ± 2 | 0 | 0 |
| | | 23-104, 24-86 | 68 ± 10 | 50 ± 6 | 31 ± 5 |
| | | 23-104, 4-25 | 21 ± 8 | 0 | 0 |
| | | 23-104, 6-119 | n.d. | n.d. | n.d. |
| | B39 | 23-104 | 0 | 0 | 0 |
| | | 23-104, 22-88 | 27 ± 2 | 0 | 0 |
| | | 23-104, 24-86 | 85 ± 1 | 52 ± 5 | 40 ± 10 |
| | | 23-104, 4-25 | 35 ± 2 | 4 ± 1 | 0 |
| | | 23-104, 6-119 | n.d. | n.d. | n.d. |
| | IGF1R3 | 23-104 | 0 | 0 | 0 |
| | | 23-104, 22-88 | 0 | 0 | 0 |
| | | 23-104, 24-86 | 0 | 0 | 0 |
| | | 23-104, 4-25 | 9 ± 4 | 0 | 0 |
| | | 23-104, 6-119 | 0 | 0 | 0 |
| | IGF1R4 | 23-104 | 2 ± 1 | 0 | 0 |
| | | 23-104, 22-88 | 3 ± 1 | 0 | 0 |
| | | 23-104, 24-86 | 0 | 0 | 0 |
| | | 23-104, 4-25 | 4 ± 2 | 2 ± 1 | 1 ± 1 |
| | | 23-104, 6-119 | 0 | 0 | 0 |
| | IGF1R5 | 23-104 | 0 | 0 | 0 |
| | | 23-104, 22-88 | 0 | 0 | 0 |
| | | 23-104, 24-86 | n.d. | n.d. | n.d. |
| | | 23-104, 4-25 | n.d. | n.d. | n.d. |
| | | 23-104, 6-119 | 61 ± 1 | 52 ± 2 | 43 ± 4 |
| | EG2 | 23-104 | 0 | 0 | 0 |
| | | 23-104, 22-88 | n.d. | n.d. | n.d. |
| | | 23-104, 24-86 | 58 ± 4 | 8 ± 5 | 1 ± 1 |
| | | 23-104, 4-25 | 40 ± 3 | 5 ± 1 | 0 |
| | | 23-104, 6-119 | 41 ± 2 | 33 ± 2 | 23 ± 1 |
| | FC5 | 23-104 | 0 | 0 | 0 |
| | | 23-104, 22-88 | n.d. | n.d. | n.d. |
| | | 23-104, 24-86 | 85 ± 5 | 39 ± 8 | 18 ± 4 |
| | | 23-104, 4-25 | 14 ± 1 | 0 | 0 |
| | | 23-104, 6-119 | 78 ± 2 | 55 ± 1 | 49 ± 2 |

Results represent the means ± SEM of three independent experiments.
N.d., not determined.

### Presence of Novel Non-Canonical Disulfide Linkages in Human Ig V_{H} Domain Repertoires.

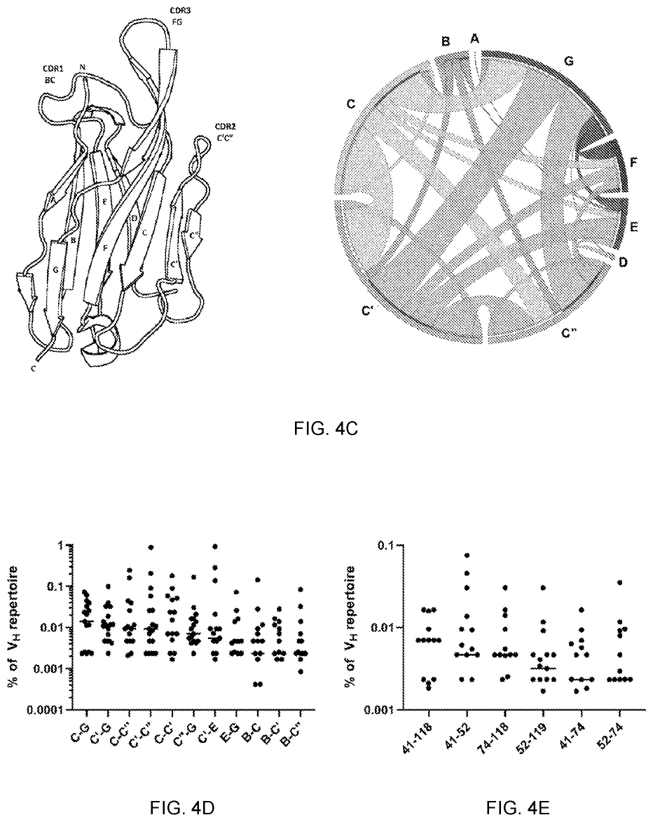

To determine whether the putative non-canonical disulfide linkages identified herein were present in the human antibody repertoire, Illumina MiSeq® sequencing was used to interrogate the peripheral blood resting memory B-cell V_{H} repertoires of 17 individuals to an average depth of 105 reads (Table 6). Cys residues in Ig V_{H} domains were most common in CDR-H3 (FIG. 4A), and V_{H}s bearing an additional non-canonical Cys pair located in FRs were rare in the repertoire, representing <1% of all rearranged V_{H}s (FIG. 4B). No V_{H} sequences were identified bearing putative non-canonical disulfide linkages formed between Cys22-Cys88, Cys24-86, Cys4-25, Cys6-119, or any of the other positions identified above. Non-canonical Cys pairs in FRs were distributed widely across various V_{H} domain strands in locations unlikely to enable disulfide linkage formation due to distance in three-dimensional space (FIG. 4C-E). These results agree with those of a recent repertoire sequencing study showing that putative non-canonical disulfide linkages formed between Cys residues in FRs were also not detected in a recent repertoire sequencing study (28). Instead, most non-canonical Cys residues in the human V_{H} repertoire lie in FR2 and FR3, where they may be able to form disulfide linkages with Cys residues in CDRs.

TABLE 6
Metrics for Illumina MiSeq ® data analyzed in this study.

| V_{H} | No. of | Raw | Merged | Quality |
|---|---|---|---|---|
| Repertoire | cells | reads | reads | filtered reads |
| 1B | 100,000 | 77,674 | 47,953 | 42,642 |
| 2B | 100,000 | 175,818 | 95,253 | 83,402 |
| 3B | 100,000 | 271,896 | 177,043 | 154,871 |
| 4B | 100,000 | 163,156 | 101,691 | 90,159 |
| 6B | 100,000 | 86,733 | 19,520 | 17,018 |
| 7B | 100,000 | 149,953 | 105,675 | 93,280 |
| 9B | 100,000 | 102,704 | 93,733 | 79,950 |
| HIV2 | 480,000 | 30,972 | 26,975 | 20,621 |
| HIV8 | 450,000 | 26,344 | 22,450 | 16,976 |
| HIV14 | 360,000 | 144,466 | 126,467 | 95,368 |
| HIV26 | 390,000 | 266,904 | 232,239 | 175,471 |
| HIV32 | 150,000 | 209,935 | 185,650 | 141,612 |
| HIV44 | 400,000 | 77,624 | 64,820 | 48,878 |
| HIV50 | 110,000 | 379,716 | 306,398 | 236,380 |
| HIV56 | 360,000 | 164,313 | 123,338 | 31,390 |
| HIV62 | 500,000 | 117,838 | 75,869 | 54,704 |
| HIV68 | 340,000 | 150,029 | 112,822 | 87,754 |

### Confirmation of the Presence of Non-Canonical Disulfide Linkages by Mass Spectrometry.

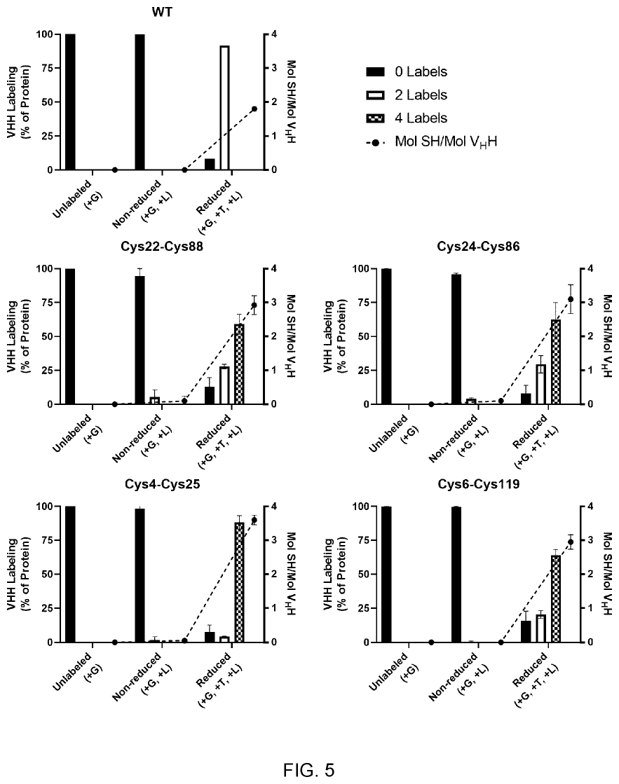

Disulfide linkage formation in Cys-engineered Ig V domains was evaluated by measuring the abundance of free sulhydryl groups with liquid chromatography-mass spectrometry (LC-MS). WT and Cys-engineered V_{H}Hs bearing putative non-canonical disulfide linkages formed between Cys22-Cys88, Cys24-86, Cys4-25, and Cys6-119 were denatured in 6 M guanidine-HCl, reduced with 5 mM tris(2-carboxyethyl)phosphine (TCEP), and then labeled with a 150-fold molar excess of maleimide-PEG2-biotin. Non-reduced (guanidine-HCl+mPEG2-biotin) and unlabeled (guanidine-HCl only) proteins served as controls. Intact LC-MS analysis confirmed the presence of all four non-canonical disulfide linkages in Ig V domains, as shown by incorporation of only two maleimide-PEG2-biotin labels in a WT V_{H}H bearing only the canonical Cys23-Cys104 disulfide linkage and up to four labels in all of the Cys-engineered V_{H}Hs following TCEP reduction (FIG. 5 and Table 7). As observed previously for the Cys54-Cys78 and Cys40-Cys55 non-canonical disulfide linkages, there was some evidence that the four novel non-canonical disulfide linkages were formed heterogeneously following introduction into V_{H}Hs bearing the canonical Cys23-Cys104 linkage. Unlike formation of the canonical Cys23-Cys104 linkage in a WT V_{H}H or formation of the Cys4-Cys25 and Cys6-Cys119 non-canonical disulfide linkages, minor proportions of V_{H}Hs bearing the Cys22-Cys88 and Cys24-Cys86 linkages (1.0-12.7%) had two free sulfhydryl groups (indicating the presence of unpaired Cys residues) without reduction. Moreover, reduction of the canonical Cys23-Cys104 disulfide linkage in a WT V_{H}H as well as reduction of the non-canonical Cys4-Cys25 disulfide linkage in Cys-engineered V_{H}Hs was ~90% efficient, while the Cys22-Cys88, Cys24-Cys86, and Cys6-Cys119 non-canonical disulfide linkages were not fully reduced in a subpopulation of V_{H}Hs under the conditions tested.

TABLE 7
Intact LC-MS analysis of free sulfhydryl abundance in wild-type and Cys-
engineered V_{H}Hs

| | | | | | | | | | Mol SH |
|---|---|---|---|---|---|---|---|---|---|
| Ig V | | | | | | | | | per |
| disulfide | | | No. of labels (%) | | | | | | mol |
| linkages | Protein | Treatment | 0 | 1 | 2 | 3 | 4 | 5 | protein |
| n.a. | β- | Unlabeled (GdnHCl) | 100.0 | — | — | — | — | — | 0.0 |
| | lactoglobulin | Nonreduced | — | 99.7 | — | 0.3 | — | — | 1.0 |
| | | (GdnHCl, m-PEG2- | | | | | | | |
| | | biotin) | | | | | | | |
| | | Reduced | — | 2.4 | — | 17.5 | — | 80.1 | 4.6 |
| | | (GdnHCl, TCEP, m- | | | | | | | |
| | | PEG2-biotin) | | | | | | | |
| Cys23- | EG2 WT | Unlabeled (GdnHCl) | 100.0 | — | — | — | — | — | 0.0 |
| Cys104 | | Nonreduced | 99.8 | — | 0.2 | — | — | — | 0.0 |
| | | (GdnHCl, m-PEG2- | | | | | | | |
| | | biotin) | | | | | | | |
| | | Reduced | 8.3 | — | 91.7 | — | — | — | 1.8 |
| | | (GdnHCl, TCEP, m- | | | | | | | |
| | | PEG2-biotin) | | | | | | | |
| Cys23- | EG2^{C22-C88} | Unlabeled (GdnHCl) | 100.0 | — | — | — | — | — | 0.0 |
| Cys104, | (ΔTₘ | Nonreduced | 94.8 | — | 5.2 | — | — | — | 0.1 |
| Cys22- | 10.6 °C) | (GdnHCl, m-PEG2- | | | | | | | |
| Cys88 | | biotin) | | | | | | | |
| | | Reduced | 8.8 | — | 28.3 | — | 62.9 | — | 3.1 |
| | | (GdnHCl, TCEP, m- | | | | | | | |
| | | PEG2-biotin) | | | | | | | |
| | IGF1R3^{C22-C88} | Unlabeled | 100.0 | — | — | — | — | — | 0.0 |
| | (ΔTₘ | (GdnHCl) | | | | | | | |
| | 8.8 °C) | Nonreduced | 87.2 | — | 12.7 | 0 | 0 | 0 | 0.3 |
| | | (GdnHCl, m-PEG2- | | | | | | | |
| | | biotin) | | | | | | | |
| | | Reduced | 16.5 | 0.2 | 25.7 | 0.4 | 57.3 | — | 2.8 |
| | | (GdnHCl, TCEP, m- | | | | | | | |
| | | PEG2-biotin) | | | | | | | |
| | FC5^{C22-C88} | Unlabeled | 100.0 | — | — | — | — | — | 0.0 |
| | (ΔTₘ | (GdnHCl) | | | | | | | |
| | 4.0 °C) | Nonreduced | 98.9 | — | 1.0 | — | — | — | 0.0 |
| | | (GdnHCl, m-PEG2- | | | | | | | |
| | | biotin) | | | | | | | |
| | | Reduced | 20.6 | — | 29.8 | — | 49.7 | — | 2.6 |
| | | (GdnHCl, TCEP, m- | | | | | | | |
| | | PEG2-biotin) | | | | | | | |
| | A5.1^{C22-C88} | Unlabeled | 100.0 | — | — | — | — | — | 0.0 |
| | (ΔTₘ | (GdnHCl) | | | | | | | |
| | 3.0 °C) | Nonreduced | 97.8 | — | 2.2 | — | — | — | 0.0 |
| | | (GdnHCl, m-PEG2- | | | | | | | |
| | | biotin) | | | | | | | |
| | | Reduced | 5.9 | — | 27.5 | — | 66.6 | — | 3.2 |
| | | (GdnHCl, TCEP, m- | | | | | | | |
| | | PEG2-biotin) | | | | | | | |
| Cys23- | EG2^{C24-C86} | Unlabeled (GdnHCl) | 99.9 | — | 0.1 | — | — | — | 0.0 |
| Cys104, | (ΔTₘ | Nonreduced | 95.4 | — | 4.5 | — | — | — | 0.1 |
| Cys24- | 12.7 °C) | (GdnHCl, m-PEG2- | | | | | | | |
| Cys86 | | biotin) | | | | | | | |
| | | Reduced | 3.7 | 0.1 | 24.9 | — | 71.3 | — | 3.4 |
| | | (GdnHCl, TCEP, m- | | | | | | | |
| | | PEG2-biotin) | | | | | | | |
| | IGF1R3^{C24-C86} | Unlabeled (GdnHCl) | 100.0 | — | — | — | — | — | 0.0 |
| | (ΔTₘ | Nonreduced | 96.5 | — | 3.5 | — | — | — | 0.1 |
| | 11.8 °C) | (GdnHCl, m-PEG2- | | | | | | | |
| | | biotin) | | | | | | | |
| | | Reduced | 12.2 | — | 34.0 | — | 53.8 | — | 2.8 |
| | | (GdnHCl, TCEP, m- | | | | | | | |
| | | PEG2-biotin) | | | | | | | |
| Cys23- | FC5^{C4-C25} | Unlabeled (GdnHCl) | 100.0 | — | — | — | — | — | 0.0 |
| Cys104, | (ΔTₘ | Nonreduced | 100.0 | — | — | — | — | — | 0.0 |
| Cys4- | 11.7 °C) | (GdnHCl, m-PEG2- | | | | | | | |
| Cys25 | | biotin) | | | | | | | |
| | | Reduced | 4.2 | — | 4.4 | — | 91.7 | — | 3.7 |
| | | (GdnHCl, TCEP, m- | | | | | | | |
| | | PEG2-biotin) | | | | | | | |
| | IGF1R4^{C4-C25} | Unlabeled (GdnHCl) | 100.0 | — | — | — | — | — | 0.0 |
| | (ΔTₘ | Nonreduced | 96.5 | — | 3.5 | — | — | — | 0.1 |
| | 7.3 °C) | (GdmCI, m-PEG2- | | | | | | | |
| | | biotin) | | | | | | | |
| | | Reduced | 11.2 | — | 4.2 | — | 84.7 | — | 3.5 |
| | | (GdnHCl, TCEP, m- | | | | | | | |
| | | PEG2-biotin) | | | | | | | |
| Cys23- | FC5^{C6-C119} | Unlabeled (GdnHCl) | 99.9 | — | 0.1 | — | — | — | 0.0 |
| Cys104, | (ΔTₘ | Nonreduced | 99.8 | — | 0.2 | — | — | — | 0.0 |
| Cys6- | 7.3 °C) | (GdnHCl, m-PEG2- | | | | | | | |
| Cys119 | | biotin) | | | | | | | |
| | | Reduced | 10.7 | — | 22.4 | — | 66.9 | — | 3.1 |
| | | (GdnHCl, TCEP, m- | | | | | | | |
| | | PEG2-biotin) | | | | | | | |
| | IGF1R4^{C6-C119} | Unlabeled (GdnHCl) | 100.0 | — | — | — | — | — | 0.0 |
| | (ΔTₘ | Nonreduced | 99.1 | — | 0.8 | — | — | — | 0.0 |
| | 11.6 °C) | (GdnHCl, m-PEG2- | | | | | | | |
| | | biotin) | | | | | | | |
| | | Reduced | 20.7 | — | 18.4 | 0.1 | 60.8 | — | 2.8 |
| | | (GdnHCl, TCEP, m- | | | | | | | |
| | | PEG2-biotin) | | | | | | | |

Abbreviations:
GdnCl, guanidine-HCl;
m-PEG2-biotin, maleimide-PEG2-biotin;
TCEP, tris(2-carboxyethyl)phosphine;
n.a., not applicable;
WT, wild-type.

### Discussion

The roles of intradomain disulfide linkages in Ig V domains are poorly understood. Even the canonical Cys23-Cys104 linkages present in all V_{H} and V_{L} domains are often dispensable for antigen binding and domain stability (29). The roles of non-canonical disulfide linkages, which typically involve one or more Cys residue(s) in CDRs, are even less clear. Such linkages are often germline encoded, with at least one Cys located in V, D, or J gene segments, and are present at relatively high frequencies in the Ig V domain repertoires of several non-human vertebrates, where they may play roles in expanding paratope diversity (15,16), structuring ultra-long CDR-H3 loops (30), reducing entropic penalties for antigen binding (31), and stabilizing the Ig V domain fold under extreme conditions (32). Of the three previously described intradomain non-canonical Ig V disulfide linkages in FRs (Cys54-Cys78, Cys40-Cys55, and Cys39-Cys87), Cys54-Cys78 was the result of somatic hypermutation in a camelid V_{H}H (7), Cys40-Cys55 is encoded in the germline of several rabbit IGHV genes (8), and Cys39-Cys87 does not occur naturally (7). Thus, non-canonical disulfide linkages in FRs appear to occur naturally more rarely than inter-CDR loop disulfide linkages even though their impacts on Ig V domain biophysical properties can be dramatic.

Here, the sequence tolerance of Ig V domains to introduction of non-canonical disulfide linkages in FRs was explored. A Cys pair scanning phage library approach was used to identify such linkages (FIG. 1), reasoning that non-canonical disulfide linkages within FRs would be generalizable to a broader spectrum of Ig V domains than those formed between Cys residues in CDRs.

Screening of the Cys pair scan V_{H} libraries identified seven novel Cys pairs putatively forming non-canonical disulfide linkages in FRs (inter-β-sheet: Cys4-Cys25, Cys22-Cys88, Cys24-Cys86 and Cys45-Cys100; intra-β-sheet: Cys4-Cys118, Cys5-Cys120 and Cys6-Cys119). Four of these seven putative non-canonical linkages (Cys22-Cys88, Cys24-Cys86, Cys4-Cys25, and Cys6-Cys119) showed significant enhancement of Tₘ and/or protease resistance when incorporated into panels of sdAbs bearing the canonical Cys23-Cys104 disulfide linkage (FIGS. 2, 3). Both the Cys4-Cys119 and Cys6-119 non-canonical linkages were previously shown to improve the thermostability of Ig C domains (5), and although Cys4-Cys119 was not identified in our screens and its impact on Ig V domain biophysical properties was not assessed, it is presumed that this linkage can form in Ig V domains as well. Intriguingly, other Cys pairs were identified whose introduction was tolerated nearby on strands A-G (Cys4-Cys118 and Cys5-Cys120); although these had only marginal effects on Tₘ, this may reflect a hotspot for disulfide linkage formation, and other combinations of Cys residues in this region may be capable of forming non-canonical disulfide linkages. Introduction of the seven Cys pairs identified in our screens into sdAbs was generally well tolerated, with minimal detrimental impact on antigen binding affinity with the potential exception of Cys4-Cys118, consistent with the location of the Trp118→Cys substitution at the base of the CDR3 loop. Introduction of all seven Cys pairs also resulted in modest decreases in the monomericity and thermal refolding efficiency of V_{H}Hs and more variable effects on the monomericity and thermal refolding of V_{H}s and V_{L}s. These parameters are much more variable for sometimes-labile V_{H} and V_{L} domains, and that the non-canonical disulfide linkages identified in this study most likely have similar effects on all Ig V domains.

Interrogation of the expressed human V_{H} repertoires of 17 individuals using next-generation DNA sequencing did not detect any of the seven putative non-canonical disulfide linkages identified from our screens at any appreciable frequency (FIG. 4). Among the rare V_{H}S bearing two non-canonical Cys residues in FRs (<1% of the repertoire), Cys pairs were widely distributed across β-strands and three-dimensional space, suggesting that their role did not involve disulfide linkage formation with one another. These FR Cys residues may instead form disulfide linkages with Cys in CDRs or may simply reflect somatic hypermutation of FRs. Interestingly, Cys residues were identified at relatively high frequencies at the N-terminus of strand G (positions 118/119), supporting the hypothesis that this region may be a hotspot for Cys residues and non-canonical disulfide linkage formation within Ig V domain FRs. A caveat to our repertoire sequencing studies was that non-canonical Cys residues could not be detected in the N-terminus of strand A or the C-terminus of strand G because these regions were primer-forced. Nevertheless, it is concluded that as with the previously described Cys39-Cys87 linkage, the novel Ig V domain non-canonical disulfide linkages described here are not present in the human repertoire despite their ability to enhance domain biophysical properties.

In summary, it was found that Ig V domain FRs were unexpectedly permissive to non-canonical disulfide linkages at multiple positions, absent in the human repertoire, whose presence was compatible with domain folding and antigen recognition while improving their thermostability and, in some cases, pepsin resistance. The results of this study more than doubles the previously known spectrum of non-canonical disulfide linkages discovered in Ig V domains, the majority of which were identified in naturally occurring antibodies. Because individual Ig V domains may differ in their tolerance of specific non-canonical disulfide linkages, this expanding panel of linkages provides a useful toolbox for stability engineering of antibodies. Clearly, the strong conservation of the canonical Cys23-Cys104 linkage does not preclude formation of other non-canonical linkages in Ig V domain FRs. This work also demonstrates the power of molecular engineering and library approaches to surpass the limitations of natural antibody repertoires.

### Experimental Procedures

Cloning of V_{H} genes into fd-tetGIIID.

V_{H} genes were cloned into the fd-tetGIIID vector (33) using standard molecular biology techniques. Genes encoding Cys23-Cys104 null mutants of V_{H}S were synthesized and cloned into pSJF2H by Genscript (Piscataway, NJ). Briefly, genes encoding VHB82, VH413 and VHM81 and their Cys23-Cys104 null derivatives were PCR-amplified from the pSJF2H vector using primers that introduced 5′ ApaL1 and 3′ Notl sites (Table 8). The amplicons and fd-tetGIIID were double-digested with ApaL1 and Notl (New England Biolabs, Ipswich, MA) and purified from 1.2% agarose gels using a QIAquick® gel extraction kit (QIAGEN, Hilden, Germany). Each insert (500 ng) was ligated with 100 ng of dephosphorylated fd-tetGIIID in 20-μL reactions containing 1 U T4 DNA ligase (Life Technologies, Carlsbad, CA) at 4 °C overnight. Electrocompetent Escherichia coli TG1 cells were transformed with the ligation mixtures by electroporation and plated on 2×YT agar plates containing 12.5 μg/mL tetracycline (Sigma, St. Louis, MO). Following overnight incubation at 37 °C, single colonies harboring fd-tetGIIID vectors with V_{H} inserts were identified by colony PCR and DNA sequencing.

TABLE 8
Primers for subcloning of VHS from pSJF2H into fd-tetGIIID.

| | | SEQ ID | |
|---|---|---|---|
| Name | Sequence (5′-3′) | NO: | Purpose |
| VHM81-F | catgcagtgcacaggaagtgcagctggtgcag | 608 | Subcloning of |
| VHM81-R | catgcagcggccgctttggaaccagagcctgaaga | 609 | VHM81 and |
| | gacggtgaccattg | | VHM81^{C23-C104 null} |
| VHB82-F | catgcagtgcacaggtgcagctgcaggag | 610 | Subcloning of |
| VHB82-R | catgcagcggccgctttggaaccagagcctgagga | 611 | VHB82 and |
| | gacggtgaccgtg | | VHB82^{C23-C104 null} |
| VH413-F | catgcagtgcacaggtccagctggtgcag | 612 | Subcloning of |
| VH413-R | catgcagcggccgctttggaaccagagcctgaaga | | VH413 and |
| | gacggtgaccattg | 613 | VH413^{C23-C104 null} |
| cPCR-F | gtgaaaaaattattattcgcaattcct | 614 | Colony PCR |
| cPCR-R | ccctcatagttagcgtaacg | 615 | Colony PCR |

Restriction enzyme recognition sequences are shown in bold underlined text.

Preparation of dUTP-containing fd-tetGIIID-VH413^{C23-C104 null} ssDNA (dU-ssDNA)

dU-ssdDNA was prepared as previously described with modifications (34,35). A single E. coli CJ236 (dut-ung) was picked from a 2×YT agar plate containing 15 μg/mL chloramphenicol (Sigma) and used to inoculate a 5 mL 2×YT culture. The culture was grown at 37 °C with 240 rpm shaking until an OD₆₀₀ of 0.5 was reached, then cells (0.1 mL) were infected with fd-tetGIIID-VH413^{C23-C104 null} phage (10¹² cfu/mL, 10 μL) for 30 min at 37 °C without shaking and plated on 2×YT agar plates containing 12.5 μg/mL tetracycline. Following overnight incubation at 37 °C, a 10 mL 2×YT culture containing tetracycline (12.5 μg/mL) and chloramphenicol (15 μg/mL) was inoculated with a single colony and grown at 37 °C with 240 rpm shaking until an OD₆₀₀ of 1.0 was reached. Two 0.5 L 2×YT cultures containing the same antibiotics and 0.25 μg/mL uridine (Sigma) were inoculated with the starter culture and grown at 37 °C with 240 rpm shaking. The next day, the cells were pelleted by centrifugation at 3,000×g/4 °C for 15 min and phage were purified by polyethylene glycol precipitation (36). Successful incorporation of uridine into phage ssDNA was verified by comparative titering of the phage on E. coli TG1 and CJ236 cells. Finally, dU-ssDNA was extracted using the QIAprep Spin M13 Kt (QIAGEN) and resuspended in water. DNA was quantitated via absorbance at 260 nm using a ND-2000 spectrophotometer (Thermo-Fisher, Waltham, MA).

Generation of fd-tetGIIID-VH413^{C23-C104 null} Cys Mutants

fd-tetGIIID-VH413^{C23-C104 null} was engineered to bear non-canonical disulfide linkages formed between Cys54-Cys78 and Cys40-Cys55 using Kunkel mutagenesis as previously described with modifications (34,35). In addition, a negative control Cys pair was introduced at locations tolerant to Cys residue introduction but known not to permit disulfide linkage formation (Cys54-Cys87). Briefly, 0.1 μg (~10 μmol) each mutagenic oligonucleotide (Table 9) was phosphorylated for 2 h at 37 °C in a 10-μL reaction containing 50 mM Tris-HCl, pH 7.5, 10 mM MgCl₂, 5 mM dithiothreitol (DTT), 0.5 mM ATP and 2.5 U T4 polynucleotide kinase (New England Biolabs). Subsequently, 20 μL of each phosphorylated oligonucleotide pair (10 μL of each oligonucleotide) were annealed with 330 ng of fd-tetGIIID-VH413^{C23-C104 null}dU-ssDNA in a 50-μL reaction whose temperature was cycled as follows: 90 °C, 3 min, 50 °C, 3 min, and 20 °C, 5 min. The annealed oligonucleotides were extended by adding 0.5 μL of 10 mM ATP, 1 μL of 10 mM dNTPs, 1 μL of 100 mM DTT, 2.5 U of T7 DNA polymerase (New England Biolabs) and 1.25 U of T4 DNA ligase (New England Biolabs) and letting covalently closed circular (CCC) heteroduplex DNA form at room temperature overnight. Formation of CCC heteroduplex DNA was verified by agarose gel electrophoresis. The reaction mixtures were purified using the QIAquick® PCR purification kit (QIAGEN) and DNA was quantitated via absorbance at 260 nm using a ND-2000 spectrophotometer. Electrocompetent E. coli TG1 cells were transformed with the CCC heteroduplex DNA by electroporation and plated on 2×YT agar plates containing 12.5 μg/mL tetracycline. Following overnight incubation at 37 °C, single colonies harboring fd-tetGIIID vectors encoding V_{H}S with the desired mutations were identified by DNA sequencing.

TABLE 9
Primers for Kunkel mutagenesis of fd-tetGIIID-VH413^{C23-C104 null}

| | | SEQ ID | |
|---|---|---|---|
| Name | Sequence (5′-3′) | NO: | Purpose |
| 54-Cys | accgctataaataaagcagacccactccagtcc | 603 | Engineering of |
| 78-Cys | ggaattgtctctggagcaggtgaatcggccc | 604 | C54-C78 into |
| | | | VH413^{C23-C104 null} |
| 40-Cys | agcctggcggacccagcacatgtagtagtcactg | 605 | Engineering of |
| 55-Cys | accaccgctataaatgcatgagacccactccag | 606 | C40-C55 into |
| | | | VH413^{C23-C104 null} |
| 54-Cys | accgctataaataaagcagacccactccagtcc | 603 | Engineering of |
| 87-Cys | gttcatttgaagatagcacgtgttcttggaattg | 607 | C54-C87 into |
| | | | VH413^{C23-C104 null} |

### Generation of Cys Pair Scan V_{H} Libraries.

The 17 Cys pair scan phage displayed libraries of fd-tetGIIID-VH413^{C23-C104 null} were generated by Kunkel mutagenesis using oligonucleotide pairs targeting specific β-strands essentially as described above. The sequences of all mutagenic oligonucleotides are provided in the sequence listing, with additional information provided in Table 10. For each library, the number of mutagenesis reactions was a×b, where a and b are the respective numbers of residues in the two β-strands being targeted. Mutagenic oligonucleotides (0.1 μg) were phosphorylated as described above, then phosphorylated oligonucleotides (either pairs of oligonucleotides each mutating one β-strand, or a single oligonucleotide mutating two β-strands; 10 μL each) were annealed with 330 ng of fd-tetGIIID-VH413^{C23-C104 null} dU-ssDNA in 50-μL reactions prepared in 96-well plates. T7 DNA polymerase and T4 DNA ligase were added to the reactions and the annealed oligonucleotides were extended overnight at room temperature to form CCC heteroduplex DNA. The individual CCC heteroduplex DNAs were pooled for each library and 25% of the total volume was purified using the QIAquick® PCR purification kit and quantitated via absorbance at 260 nm using a ND-2000 spectrophotometer. Electrocompetent E. coli TG1 cells were transformed with the CCC heteroduplex DNA by electroporation (five transformations per library) and plated on 2×YT agar plates containing 12.5 μg/mL tetracycline. Following overnight incubation at 37 °C, 40-50 single colonies per library harboring fd-tetGIIID vectors encoding V_{H}S were sequenced to determine the proportion correctly mutagenized and the functional library size.

TABLE 10
Sequences of mutagenic oligonucleotides used
to construct 17 Cys pair scan phage-displayed
fd-tetGIIID-VH413^{C23-C104 null} libraries.

| | SEQ | |
|---|---|---|
| | ID | β- |
| Oligo | NO: | strand |
| A1 | 1 | A |
| A2 | 2 | A |
| A3 | 3 | A |
| A4 | 4 | A |
| A5 | 5 | A |
| A6 | 6 | A |
| A7 | 7 | A |
| A8 | 8 | A |
| A9 | 9 | A |
| A10 | 10 | A |
| A11 | 11 | A |
| A12 | 12 | A |
| A13 | 13 | A |
| A14 | 14 | A |
| B1 | 15 | B |
| B2 | 16 | B |
| B3 | 17 | B |
| B4 | 18 | B |
| B5 | 19 | B |
| B6 | 20 | B |
| B7 | 21 | B |
| B8 | 22 | B |
| B9 | 23 | B |
| B10 | 24 | B |
| B11 | 25 | B |
| C1 | 26 | C |
| C2 | 27 | C |
| C3 | 28 | C |
| C4 | 29 | C |
| C5 | 30 | C |
| C6 | 31 | C |
| C7 | 32 | C |
| C8 | 33 | C |
| C20 | 35 | C |
| C01 | 40 | C′ |
| C02 | 41 | C′ |
| C03 | 42 | C′ |
| C04 | 43 | C′ |
| C05 | 44 | C′ |
| C06 | 45 | C′ |
| C07 | 46 | C′ |
| C08 | 47 | C′ |
| C09 | 48 | C′ |
| C201 | 49 | C″ |
| C202 | 50 | C″ |
| C203 | 51 | C″ |
| C204 | 52 | C″ |
| C205 | 53 | C″ |
| C206 | 54 | C″ |
| C207 | 55 | C″ |
| C208 | 56 | C″ |
| D1 | 57 | D |
| D2 | 58 | D |
| D3 | 59 | D |
| D4 | 60 | D |
| D5 | 61 | D |
| D6 | 62 | D |
| D7 | 63 | D |
| D8 | 64 | D |
| D9 | 65 | D |
| D10 | 66 | D |
| E1 | 67 | E |
| E2 | 68 | E |
| E3 | 69 | E |
| E4 | 70 | E |
| E5 | 71 | E |
| E6 | 72 | E |
| E7 | 73 | E |
| E8 | 74 | E |
| E9 | 75 | E |
| E10 | 76 | E |
| E11 | 77 | E |
| E12 | 78 | E |
| F1 | 79 | F |
| F2 | 80 | F |
| F3 | 81 | F |
| F4 | 82 | F |
| F5 | 83 | F |
| F6 | 84 | F |
| F7 | 85 | F |
| F8 | 86 | F |
| G1 | 87 | G |
| G2 | 88 | G |
| G3 | 89 | G |
| G4 | 90 | G |
| G5 | 91 | G |
| G6 | 92 | G |
| G7 | 93 | G |
| G8 | 94 | G |
| G9 | 95 | G |
| G10 | 96 | G |
| G11 | 97 | G |
| A6-B1 | 98 | A-B |
| A7-B1 | 99 | A-B |
| A7-B2 | 100 | A-B |
| A8-B1 | 101 | A-B |
| A8-B2 | 102 | A-B |
| A8-B3 | 103 | A-B |
| A9-B1 | 104 | A-B |
| A9-B2 | 105 | A-B |
| A9-B3 | 106 | A-B |
| A9-B4 | 107 | A-B |
| A10-B1 | 108 | A-B |
| A10-B2 | 109 | A-B |
| A10-B3 | 110 | A-B |
| A10-B4 | 111 | A-B |
| A10-B5 | 112 | A-B |
| A11-B1 | 113 | A-B |
| A11-B2 | 114 | A-B |
| A11-B3 | 115 | A-B |
| A11-B4 | 116 | A-B |
| A11-B5 | 117 | A-B |
| A11-B6 | 118 | A-B |
| A12-B1 | 119 | A-B |
| A12-B2 | 120 | A-B |
| A12-B3 | 121 | A-B |
| A12-B4 | 122 | A-B |
| A12-B5 | 123 | A-B |
| A12-B6 | 124 | A-B |
| A12-B7 | 125 | A-B |
| A13-B1 | 126 | A-B |
| A13-B2 | 127 | A-B |
| A13-B3 | 128 | A-B |
| A13-B4 | 129 | A-B |
| A13-B5 | 130 | A-B |
| A13-B6 | 131 | A-B |
| A13-B7 | 132 | A-B |
| A13-B8 | 133 | A-B |
| A14-B1 | 134 | A-B |
| A14-B2 | 135 | A-B |
| A14-B3 | 136 | A-B |
| A14-B4 | 137 | A-B |
| A14-B5 | 138 | A-B |
| A14-B6 | 139 | A-B |
| A14-B7 | 140 | A-B |
| A14-B8 | 141 | A-B |
| A14-B9 | 142 | A-B |
| B11-C1 | 143 | B-C |
| C11 | 144 | C-C′ |
| C12 | 145 | C-C′ |
| C13 | 146 | C-C′ |
| C21 | 147 | C-C′ |
| C22 | 148 | C-C′ |
| C23 | 149 | C-C′ |
| C24 | 150 | C-C′ |
| C31 | 151 | C-C′ |
| C32 | 152 | C-C′ |
| C33 | 153 | C-C′ |
| C34 | 154 | C-C′ |
| C35 | 155 | C-C′ |
| C41 | 156 | C-C′ |
| C42 | 157 | C-C′ |
| C43 | 158 | C-C′ |
| C44 | 159 | C-C′ |
| C45 | 160 | C-C′ |
| C46 | 161 | C-C′ |
| C51 | 162 | C-C′ |
| C52 | 163 | C-C′ |
| C53 | 164 | C-C′ |
| C54 | 165 | C-C′ |
| C55 | 166 | C-C′ |
| C56 | 167 | C-C′ |
| C57 | 168 | C-C′ |
| C61 | 169 | C-C′ |
| C62 | 170 | C-C′ |
| C63 | 171 | C-C′ |
| C64 | 172 | C-C′ |
| C65 | 173 | C-C′ |
| C66 | 174 | C-C′ |
| C67 | 175 | C-C′ |
| C68 | 176 | C-C′ |
| C71 | 177 | C-C′ |
| C72 | 178 | C-C′ |
| C73 | 179 | C-C′ |
| C74 | 180 | C-C′ |
| C75 | 181 | C-C′ |
| C76 | 182 | C-C′ |
| C77 | 183 | C-C′ |
| C78 | 184 | C-C′ |
| C79 | 185 | C-C′ |
| C81 | 186 | C-C′ |
| C82 | 187 | C-C′ |
| C83 | 188 | C-C′ |
| C84 | 189 | C-C′ |
| C85 | 190 | C-C′ |
| C86 | 191 | C-C′ |
| C87 | 192 | C-C′ |
| C88 | 193 | C-C′ |
| C89 | 194 | C-C′ |
| C08-C201 | 195 | C′-C″ |
| C09-C201 | 196 | C′-C″ |
| C09-C202 | 197 | C′-C″ |
| C201-D1 | 198 | C″-D |
| C201-D2 | 199 | C″-D |
| C202-D1 | 200 | C″-D |
| C202-D2 | 201 | C″-D |
| C202-D3 | 202 | C″-D |
| C203-D1 | 203 | C″-D |
| C203-D2 | 204 | C″-D |
| C203-D3 | 205 | C″-D |
| C203-D4 | 206 | C″-D |
| C204-D1 | 207 | C″-D |
| C204-D2 | 208 | C″-D |
| C204-D3 | 209 | C″-D |
| C204-D4 | 210 | C″-D |
| C204-D5 | 211 | C″-D |
| C205-D1 | 212 | C″-D |
| C205-D2 | 213 | C″-D |
| C205-D3 | 214 | C″-D |
| C205-D4 | 215 | C″-D |
| C205-D5 | 216 | C″-D |
| C205-D6 | 217 | C″-D |
| C206-D1 | 218 | C″-D |
| C206-D2 | 219 | C″-D |
| C206-D3 | 220 | C″-D |
| C206-D4 | 221 | C″-D |
| C206-D5 | 222 | C″-D |
| C206-D6 | 223 | C″-D |
| C206-D7 | 224 | C″-D |
| C207-D1 | 225 | C″-D |
| C207-D2 | 226 | C″-D |
| C207-D3 | 227 | C″-D |
| C207-D4 | 228 | C″-D |
| C207-D5 | 229 | C″-D |
| C207-D6 | 230 | C″-D |
| C207-D7 | 231 | C″-D |
| C207-D8 | 232 | C″-D |
| C208-D1 | 233 | C″-D |
| C208-D2 | 234 | C″-D |
| C208-D3 | 235 | C″-D |
| C208-D4 | 236 | C″-D |
| C208-D5 | 237 | C″-D |
| C208-D6 | 238 | C″-D |
| C208-D7 | 239 | C″-D |
| C208-D8 | 240 | C″-D |
| C208-D9 | 241 | C″-D |
| D2-E1 | 242 | D-E |
| D3-E1 | 243 | D-E |
| D3-E2 | 244 | D-E |
| D4-E1 | 245 | D-E |
| D4-E2 | 246 | D-E |
| D4-E3 | 247 | D-E |
| D5-E1 | 248 | D-E |
| D5-E2 | 249 | D-E |
| D5-E3 | 250 | D-E |
| D5-E4 | 251 | D-E |
| D6-E1 | 252 | D-E |
| D6-E2 | 253 | D-E |
| D6-E3 | 254 | D-E |
| D6-E4 | 255 | D-E |
| D6-E5 | 256 | D-E |
| D7-E1 | 257 | D-E |
| D7-E2 | 258 | D-E |
| D7-E3 | 259 | D-E |
| D7-E4 | 260 | D-E |
| D7-E5 | 261 | D-E |
| D7-E6 | 262 | D-E |
| D8-E1 | 263 | D-E |
| D8-E2 | 264 | D-E |
| D8-E3 | 265 | D-E |
| D8-E4 | 266 | D-E |
| D8-E5 | 267 | D-E |
| D8-E6 | 268 | D-E |
| D8-E7 | 269 | D-E |
| D9-E1 | 270 | D-E |
| D9-E2 | 271 | D-E |
| D9-E3 | 272 | D-E |
| D9-E4 | 273 | D-E |
| D9-E5 | 274 | D-E |
| D9-E6 | 275 | D-E |
| D9-E7 | 276 | D-E |
| D9-E8 | 277 | D-E |
| D10-E1 | 278 | D-E |
| D10-E2 | 279 | D-E |
| D10-E3 | 280 | D-E |
| D10-E4 | 281 | D-E |
| D10-E5 | 282 | D-E |
| D10-E6 | 283 | D-E |
| D10-E7 | 284 | D-E |
| D10-E8 | 285 | D-E |
| D10-E9 | 286 | D-E |
| E4-F1 | 287 | E-F |
| E5-F1 | 288 | E-F |
| E5-F2 | 289 | E-F |
| E6-F1 | 290 | E-F |
| E6-F2 | 291 | E-F |
| E6-F3 | 292 | E-F |
| E7-F1 | 293 | E-F |
| E7-F2 | 294 | E-F |
| E7-F3 | 295 | E-F |
| E7-F4 | 296 | E-F |
| E8-F1 | 297 | E-F |
| E8-F2 | 298 | E-F |
| E8-F3 | 299 | E-F |
| E8-F4 | 300 | E-F |
| E8-F5 | 301 | E-F |
| E9-F1 | 302 | E-F |
| E9-F2 | 303 | E-F |
| E9-F3 | 304 | E-F |
| E9-F4 | 305 | E-F |
| E9-F5 | 306 | E-F |
| E9-F6 | 307 | E-F |
| E10-F1 | 308 | E-F |
| E10-F2 | 309 | E-F |
| E10-F3 | 310 | E-F |
| E10-F4 | 311 | E-F |
| E10-F5 | 312 | E-F |
| E10-F6 | 313 | E-F |
| E10-F7 | 314 | E-F |
| E11-F1 | 315 | E-F |
| E11-F2 | 316 | E-F |
| E11-F3 | 317 | E-F |
| E11-F4 | 318 | E-F |
| E11-F5 | 319 | E-F |
| E11-F6 | 320 | E-F |
| E11-F7 | 321 | E-F |
| E11-F8 | 322 | E-F |
| E12-F1 | 323 | E-F |
| E12-F2 | 324 | E-F |
| E12-F3 | 325 | E-F |
| E12-F4 | 326 | E-F |
| E12-F5 | 327 | E-F |
| E12-F6 | 328 | E-F |
| E12-F7 | 329 | E-F |
| E12-F8 | 330 | E-F |

TABLE 11
Sequences of V_{H}S, V_{H}Hs, V_{L}s and Cys-engineered variants.

| Construct | SEQ ID |
|---|---|
| Name | NO: |
| A4.2 | 331 |
| A4.2_22_88 | 332 |
| A4.2_24_86 | 333 |
| A4.2_45_100 | 334 |
| A4.2_4_25 | 335 |
| A4.2_4_118 | 336 |
| A4.2_5_120 | 337 |
| A4.2_6_119 | 338 |
| A5.1 | 339 |
| A5.1_22_88 | 340 |
| A5.1_24_86 | 341 |
| A5.1_45_100 | 342 |
| A5.1_4_25 | 343 |
| A5.1_4_118 | 344 |
| A5.1_5_120 | 345 |
| A5.1_6_119 | 346 |
| A20.1 | 347 |
| A20.1_22_88 | 348 |
| A20.1_24_86 | 349 |
| A20.1_45_100 | 350 |
| A20.1_4_25 | 351 |
| A20.1_4_118 | 352 |
| A20.1_5_120 | 353 |
| A20.1_6_119 | 354 |
| A24.1 | 355 |
| A24.1_22_88 | 356 |
| A24.1_24_86 | 357 |
| A24.1_45_100 | 358 |
| A24.1_4_25 | 359 |
| A24.1_4_118 | 360 |
| A24.1_5_120 | 361 |
| A24.1_6_119 | 362 |
| A26.8 | 363 |
| A26.8_22_88 | 364 |
| A26.8_24_86 | 365 |
| A26.8_45_100 | 366 |
| A26.8_4_25 | 367 |
| A26.8_4_118 | 368 |
| A26.8_5_120 | 369 |
| A26.8_6_119 | 370 |
| B39 | 371 |
| B39_22_88 | 372 |
| B39_24_86 | 373 |
| B39_45_100 | 374 |
| B39_4_25 | 375 |
| B39_4_118 | 376 |
| B39_5_120 | 377 |
| B39_6_119 | 378 |
| IGF1R3 | 379 |
| IGF1R3_22_88 | 380 |
| IGF1R3_24_86 | 381 |
| IGF1R3_45_100 | 382 |
| IGF1R3_4_25 | 383 |
| IGF1R3_4_118 | 384 |
| IGF1R3_5_120 | 385 |
| IGF1R3_6_119 | 386 |
| IGF1R4 | 387 |
| IGF1R4_22_88 | 388 |
| IGF1R4_24_86 | 389 |
| IGF1R4_45_100 | 390 |
| IGF1R4_4_25 | 391 |
| IGF1R4_4_118 | 392 |
| IGF1R4_5_120 | 393 |
| IGF1R4_6_119 | 394 |
| IGF1R5 | 395 |
| IGF1R5_22_88 | 396 |
| IGF1R5_24_86 | 397 |
| IGF1R5_45_100 | 398 |
| IGF1R5_4_25 | 399 |
| IGF1R5_4_118 | 400 |
| IGF1R5_5_120 | 40 |
| IGF1R5_6_119 | 402 |
| EG2 | 403 |
| EG2_22_88 | 404 |
| EG2_24_86 | 405 |
| EG2_45_100 | 406 |
| EG2_4_25 | 407 |
| EG2_4_118 | 408 |
| EG2_5_120 | 409 |
| EG2_6_119 | 410 |
| FC5 | 411 |
| FC5_22_88 | 412 |
| FC5_24_86 | 413 |
| FC5_45_100 | 414 |
| FC5_4_25 | 415 |
| FC5_4_118 | 416 |
| FC5_5_120 | 417 |
| FC5_6_119 | 418 |
| VH413 | 419 |
| VH413_22_88 | 420 |
| VH413_24_86 | 421 |
| VH413_45_100 | 422 |
| VH413_4_25 | 423 |
| VH413_4_118 | 424 |
| VH413_5_120 | 425 |
| VH413_6_119 | 426 |
| VH414 | 427 |
| VH414_22_88 | 428 |
| VH414_24_86 | 429 |
| VH414_45_100 | 430 |
| VH414_4_25 | 431 |
| VH414_4_118 | 432 |
| VH414_5_120 | 433 |
| VH414_6_119 | 434 |
| VH419 | 435 |
| VH419_22_88 | 436 |
| VH419_24_86 | 437 |
| VH419_45_100 | 438 |
| VH419_4_25 | 439 |
| VH419_4_118 | 440 |
| VH419_5_120 | 441 |
| VH419_6_119 | 442 |
| VH420 | 443 |
| VH420_22_88 | 444 |
| VH420_24_86 | 445 |
| VH420_45_100 | 446 |
| VH420_4_25 | 447 |
| VH420_4_118 | 448 |
| VH420_5_120 | 449 |
| VH420_6_119 | 450 |
| VH421 | 451 |
| VH421_22_88 | 452 |
| VH421_24_86 | 453 |
| VH421_45_100 | 454 |
| VH421_4_25 | 455 |
| VH421_4_118 | 456 |
| VH421_5_120 | 457 |
| VH421_6_119 | 458 |
| VH423 | 459 |
| VH423_22_88 | 460 |
| VH423_24_86 | 461 |
| VH423_45_100 | 462 |
| VH423_4_25 | 463 |
| VH423_4_118 | 464 |
| VH423_5_120 | 465 |
| VH423_6_119 | 466 |
| VH428 | 467 |
| VH428_22_88 | 468 |
| VH428_24_86 | 469 |
| VH428_45_100 | 470 |
| VH428_4_25 | 471 |
| VH428_4_118 | 472 |
| VH428_5_120 | 473 |
| VH428_6_119 | 474 |
| VH429 | 475 |
| VH429_22_88 | 476 |
| VH429_24_86 | 477 |
| VH429_45_100 | 478 |
| VH429_4_25 | 479 |
| VH429_4_118 | 480 |
| VH429_5_120 | 481 |
| VH429_6_119 | 482 |
| VHM41 | 483 |
| VHM41_22_88 | 484 |
| VHM41_24_86 | 485 |
| VHM41_45_100 | 486 |
| VHM41_4_25 | 487 |
| VHM41_4_118 | 488 |
| VHM41_5_120 | 489 |
| VHM41_6_119 | 490 |
| VH44 | 491 |
| VH44_22_88 | 492 |
| VH44_24_86 | 493 |
| VH44_45_100 | 494 |
| VH44_4_25 | 495 |
| VH44_4_118 | 496 |
| VH44_5_120 | 497 |
| VH44_6_119 | 498 |
| VHM81 | 499 |
| VHM81_22_88 | 500 |
| VHM81_24_86 | 501 |
| VHM81_45_100 | 502 |
| VHM81_4_25 | 503 |
| VHM81_4_118 | 504 |
| VHM81_5_120 | 505 |
| VHM81_6_119 | 506 |
| VHB82 | 507 |
| VHB82_22_88 | 508 |
| VHB82_24_86 | 509 |
| VHB82_45_100 | 510 |
| VHB82_4_25 | 511 |
| VHB82_4_118 | 512 |
| VHB82_5_120 | 513 |
| VHB82_6_119 | 514 |
| VL324 | 515 |
| VL324_22_88 | 516 |
| VL324_24_86 | 517 |
| VL324_45_100 | 518 |
| VL324_4_25 | 519 |
| VL324_4_118 | 520 |
| VL324_5_120 | 521 |
| VL324_6_119 | 522 |
| VL325 | 523 |
| VL325_22_88 | 524 |
| VL325_24_86 | 525 |
| VL325_45_100 | 526 |
| VL325_4_25 | 527 |
| VL325_4_118 | 528 |
| VL325_5_120 | 529 |
| VL325_6_119 | 530 |
| VL330 | 531 |
| VL330_22_88 | 532 |
| VL330_24_86 | 533 |
| VL330_45_100 | 534 |
| VL330_4_25 | 535 |
| VL330_4_118 | 536 |
| VL330_5_120 | 537 |
| VL330_6_119 | 538 |
| VL335 | 539 |
| VL335_22_88 | 540 |
| VL335_24_86 | 541 |
| VL335_45_100 | 542 |
| VL335_4_25 | 543 |
| VL335_4_118 | 544 |
| VL335_5_120 | 545 |
| VL335_6_119 | 546 |
| VL342 | 547 |
| VL342_22_88 | 548 |
| VL342_24_86 | 549 |
| VL342_45_100 | 550 |
| VL342_4_25 | 551 |
| VL342_4_118 | 552 |
| VL342_5_120 | 553 |
| VL342_6_119 | 554 |
| VL351 | 555 |
| VL351_22_88 | 556 |
| VL351_24_86 | 557 |
| VL351_45_100 | 558 |
| VL351_4_25 | 559 |
| VL351_4_118 | 560 |
| VL351_5_120 | 561 |
| VL351_6_119 | 562 |
| VL364 | 563 |
| VL364_22_88 | 564 |
| VL364_24_86 | 565 |
| VL364_45_100 | 566 |
| VL364_4_25 | 567 |
| VL364_4_118 | 568 |
| VL364_5_120 | 569 |
| VL364_6_119 | 570 |
| VL382 | 571 |
| VL382_22_88 | 572 |
| VL382_24_86 | 573 |
| VL382_45_100 | 574 |
| VL382_4_25 | 575 |
| VL382_4_118 | 576 |
| VL382_5_120 | 577 |
| VL382_6_119 | 578 |
| VL383 | 579 |
| VL383_22_88 | 580 |
| VL383_24_86 | 581 |
| VL383_45_100 | 582 |
| VL383_4_25 | 583 |
| VL383_4_118 | 584 |
| VL383_5_120 | 585 |
| VL383_6_119 | 586 |
| VL389 | 587 |
| VL389_22_88 | 588 |
| VL389_24_86 | 589 |
| VL389_45_100 | 590 |
| VL389_4_25 | 591 |
| VL389_4_118 | 592 |
| VL389_5_120 | 593 |
| VL389_6_119 | 594 |
| VL3103 | 595 |
| VL3103_22_88 | 596 |
| VL3103_24_86 | 597 |
| VL3103_45_100 | 598 |
| VL3103_4_25 | 599 |
| VL3103_4_118 | 600 |
| VL3103_5_120 | 601 |
| VL3103_6_119 | 602 |

Panning of Phage-Displayed V_{H}s and V_{IA} Libraries Against Protein A

To prepare fd phage displaying V_{H}s and their Cys-engineered variants (Table 11) as well as Cys pair scan libraries of VH413^{C23-C104 null} E. coli TG1 cells transformed with the heteroduplex CCC DNAs above were transferred to 100 mL 2×YT cultures containing tetracycline (12.5 μg/mL) and grown overnight at 3700 with 240 rpm shaking. The next day, phage were purified by polyethylene glycol precipitation and resuspended in 2 mL of phosphate-buffered saline (PBS), pH 7.4. Phage concentrations were estimated spectrophotometrically using the formula:

$$\frac{v i r i o n s}{m L} = \frac{( {A}_{269} - {A}_{320} ) \times 1 {0}^{16}}{No . of bases in genome}$$

Phage were aliquoted and stored at −20 °C. For panning, wells of NUNC MaxiSorp™ microtiter plates (Thermo-Fisher) were coated overnight at 400 with 5 μg of protein A in 100 μL of PBS. The next day, wells were blocked with 300 μL of PBS containing 2% (w/v) skim milk at 37 °C for 2.5 h. Single V_{H}-displaying phage clones or Cys pair scan libraries of phage-displayed V_{H}s (10¹² cfu/mL, 100 μL in PBS containing 2% skim milk) were added to wells and incubated for 1.5 h. Pooled intra- and inter-β-sheet Cys pair scan libraries were panned in duplicate using either (i) phage prepared as above or (ii) phage heated at 50 °C for 30 min, allowed to cool for 20 min at room temperature, then centrifuged at 16,000×g for 5 min prior to each round of selection. Pooled inter-β-sheet Cys pair scan libraries were panned in duplicate using phage subjected to heating and centrifugation as described. Wells were washed 15 times with PBS containing 0.05% (v/v) TWEEN®-20 (Sigma) and five times with PBS. Bound phage were eluted with 150 μL of 100 mM trimethylamine (Sigma) for 10 min with occasional mixing and neutralized with an equivalent volume of 1 M Tris-HCl, pH 7.5. Phage titers were determined by infection of mid-log phase E. coli TG1 cells (OD₆₀₀=0.5) and plating on 2×YT agar plates containing 12.5 μg/mL tetracycline. Phage were amplified by overnight growth of the infected cells in 100 mL 2×YT cultures containing tetracycline (12.5 μg/mL) at 37 °C with 240 rpm shaking and purified from culture supernatants the next day for subsequent panning rounds.
Expression of V_{H}H, V_{H} and V_{L} Domains

The coding sequences of sdAbs were synthesized and directionally inserted between the EcoRl and BamHl sites of the pSJF2H expression vector by Genscript. Following transformation of E. coli TG1 cells, single colonies were grown in 10 mL 2×YT media containing 1% glucose and 100 μg/mL ampicillin for 4 h at 37 °C with 250 rpm shaking. The pre-cultures were used to inoculate overnight cultures (37 °C, 250 mL 2×YT medium, 250 rpm shaking) or 5-day cultures (25 °C, 1 L fortified M9 minimal medium, 180 rpm shaking). Expression was induced with 0.5 mM isopropyl β-D-1-thiogalactopyranoside (IPTG) when the optical densities (600 nm) of 250 mL overnight cultures reached 0.4-0.5 and with 100 mL of 10× induction medium/0.1 mM IPTG after 30 h for 1 L M9 cultures (37). Periplasmic proteins were extracted by sucrose shock and Hiss-tagged sdAbs were purified by immobilized metal affinity chromatography on HisTrap™ HP affinity columns (Cytiva Life Sciences, Marlborough, MA) connected to an AKTA FPLC system (Cytiva Life Sciences). Protein yield and purity was assessed by spectrophotometry (280 nm) and SDS-PAGE.

### Circular Dichroism

Proteins (0.1 mg/mL) were dialyzed into 0.1 M sodium phosphate buffer the night before circular dichroism (CD) experiments. CD spectra and temperature-dependent ellipticity measurements were collected using a Jasco J-815 spectropolarimeter (Jasco, Easton, MD) equipped with a Peltier thermoelectric type temperature control system. Samples (0.2 mL) were placed into 1-mm pathlength cuvettes. Spectral scans over wavelengths from 190-250 nm were performed at 25 °C with three accumulations, a scan speed of 200 nm/min, a digital integration time (DIT) of 1 s, and a bandwidth of 1 nm. Ellipticity was measured at wavelengths of 205-210 nm over a temperature range of 25-106 °C with intervals of 0.5 °C, a ramp rate of 2 °C/min, a DIT of 2 s, and a bandwidth of 1 nm. The data were normalized to a percent scale and fitted to a Boltzmann distribution for calculation of Tₘ. To determine refolding efficiencies, the scanned samples were cooled to 25 °C and used to obtain second sigmoidal melting curves as described. Refolding efficiencies were calculated as the ratios of the normalized upper plateau ellipticity values for the second melting curves to those of the first melting curves.

### Differential Scanning Fluorometry

Differential scanning fluorometry was performed using SYPRO® Orange (Life Technologies) essentially as described previously (19). SYPRO® Orange (5 μL) was added to sdAbs (45 μL; 1 mg/mL in PBS) in wells of 96-well thin-wall optical plates. A temperature ramp rate of 1 °C/min was applied using an iQ™ 5 real-time PCR system (Bio-Rad, Hercules, CA) and thermal unfolding was monitored by measuring fluorescence (excitation and emission 490 and 575 nm, respectively) at 0.5 °C intervals. Tₘs were calculated as the temperature at which the maximum rate of change in fluorescent signal [d(RFU)/dt] was achieved.

### Size Exclusion Chromatography-Multiangle Light Scattering (SEC-MALS)

UPLC SEC-MALS was performed using an Acquity BEH-125 column (Waters, Milford, MA) connected to an Acquity UPLC™ H-Class Bio system (Waters) with miniDAWN™ MALS detector and Optilab® UT-rEX™ refractometer (Wyatt Technology, Santa Barbara, CA). The sdAbs (10-20 μg) were injected at 30 °C in a PBS mobile phase at a flow rate of 0.4 mL/min. Weighted average molecular mass (M_{MALS}) was calculated using a protein concentration determined from Δ₂₈₀ measurements and extinction coefficients calculated from amino acid sequences. Data were processed using ASTRA 6.1 software (Wyatt Technology).

### Surface Plasmon Resonance (SPR)

Prior to SPR experiments, all sdAbs were purified by size exclusion chromatography (SEC) using a Superdex™ 75 GL column (Cytiva Life Sciences) connected an AKTA FPLC system. The mobile phase for SEC consisted of HBS-EP (10 mM HEPES, pH 7.4, containing 150 mM NaCl, 3 mM ethylenediaminetetraacetic acid and 0.005% surfactant P20; Cytiva Life Sciences). EGFR [Z03194-50, Genscript; 263 response units (RUs)], IGF1R (391-GR-050, R&D Systems, Minneapolis, MN; 364 RUs), C. difficile toxin A (Cedarlane, Burlington, Canada; 4778 RUs) and C. difficile toxin B fragment (aa 1751-2366; a generous gift from Ken Ng, University of Calgary; 1246 RUs) were immobilized on sensor chips CM5 by amine coupling in 10 mM sodium acetate, pH 3.5-4.5. An ethanolamine-blocked flow cell served as a reference. Six to eight different concentrations of sdAbs (25 μM to 7.4 μM, depending on K_{D}) were injected over the surfaces at 25 °C on a Biacore®3000 instrument (Cytiva Life Sciences) at a flow rate of 20-40 μL/min with contact times of 120-300 s and dissociation times of 160-300 s. The EGFR and toxin A surfaces were regenerated with running buffer, while the IGF1R and toxin B surfaces were regenerated using 10 mM glycine, pH 1.5. Affinities were calculated by fitting the data to a 1:1 binding model or a steady-state binding model using BIAevaluation software version 4.1.1 (Cytiva Life Sciences).

For analyses of protein A and protein L binding, protein A (21184, Thermo-Fisher; 1387 RUs) and protein L (P121189, Thermo-Fisher; 637 RUs) were immobilized on CM5 sensor chips by amine coupling in 10 mM sodium acetate, pH 4.5. SEC-purified sdAbs in HBS-EP (for Biacore 3000 experiments) or HBS-EP+ (for Biacore T200 experiments; identical to HBS-EP but containing 0.05% surfactant P20). An ethanolamine- or ovalbumin-blocked flow cell served as a reference. Single concentrations of V_{H}Hs, V_{H}s (2.5-10 μM) or V_{L}s (500 nM to 3 μM) were injected over the surfaces on Biacore 3000 or T200 instruments at a flow rate of 20 μL/min with a contact time of 120 s and a dissociation time of 300 s. The surfaces were regenerated using 10 mM glycine, pH 1.5. The data were fitted to a steady state binding model using BIAevaluation Software v3.0 (Biacore T200) or v4.1.1 (Biacore 3000) and reported as yes/no binding.

### Protease Digestion Assays

Digestions with pepsin, trypsin and chymotrypsin were performed essentially as described previously (27). Briefly, titrated working concentrations of enzymes were prepared in 1 mM HCl (pepsin) or PBS containing 10 mM CaCl₂ (trypsin and chymotrypsin). Five micrograms of each sdAb were digested at 37 °C for 1 h in 20-μL reactions with a final pH of 2.0 for pepsin digestions. Pepsin digestions were neutralized with 1 M NaOH and trypsin/chymotrypsin digestions were neutralized with a protease inhibitor cocktail (Sigma). The digested sdAbs were separated by SDS-PAGE and band densitometry was conducted using ImageJ v.1.53. Three independent digestions were conducted for each V_{H}H.

### Mass Spectrometry

Analyses of free sulfhydryl abundance using LC-MS were performed essentially as previously described (38). Briefly, WT or Cys-engineered sdAbs (60 μg) were buffer exchanged into 100 mM sodium acetate, pH 5.5, containing 6 M guanidine-HCl and 20 μg was set aside as the unlabelled control. Another 20 μg was reduced with 5 mM TCEP for 15 min at 37 °C. Both the reduced and unreduced samples were then labeled with EZ-Link™ maleimide-PEG2-biotin (Thermo Fisher) for 1 h at room temperature using a 150:1 (mol label: mol protein) ratio for the unreduced samples and a 150:1 (mol label: mol Cys) ratio for the reduced samples. Samples were buffer exchanged again to remove excess label. LC-MS analysis of intact proteins was performed using a Dionex UltiMate 3000 HPLC instrument (Thermo Fisher) equipped with a POROS™ R2 (10 μm, 2.1×30 mm) column (Thermo Scientific) coupled to a LTQ-Orbitrap XL mass spectrometer equipped with an electrospray ionization source (Thermo Scientific). Approximately 5 μg of protein was injected at a flow rate of 3 mL/min with a column temperature of 80 °C. The mobile phases were 0.1% formic acid in ddH₂O (A) and acetonitrile (B). Proteins were eluted with a linear gradient of 10% to 75% mobile phase B over 3 min and split at 100 μL/min to the LTQ-Orbitrap XL. MS analysis was conducted in positive electrospray ionization mode using appropriate tune files for analysis of small intact proteins. Data was deconvoluted using the MaxEnt1 algorithm available through MassLynx software (Waters).

### Illumina MiSeq Sequencing

Sequencing of human expressed V_{H} repertoires was conducted essentially as previously described (39). The study was approved by the Simon Fraser University Research Ethics Board (#2012s0182) and abided by the principles laid out in the Declaration of Helsinki. Peripheral blood mononuclear cells were isolated from the blood of healthy and HIV⁺ individuals by density gradient centrifugation, and resting memory B cells (CD21+IgG+CD19+CD27+CD10-CD20+) were obtained by fluorescent-activated cell sorting as described (40). Total cellular RNA was extracted and reverse transcribed into cDNA, and then expressed V_{H} genes were amplified in two rounds of universal tag PCR. The resulting amplicons were pooled and purified by gel extraction followed by solid-phase reversible immobilization with AMPure® XP beads (Beckman-Coulter, Brea, CA). The pooled amplicons were quantitated using a Qubit 2.0 fluorometer (Life Technologies) and sequenced using a 500-cycle Reagent Kit V2 and a MiSeq® instrument (Illumina, San Diego, CA) with a 5% PhiX genomic DNA spike. Forward and reverse reads were merged using FLASH (41) with default parameters and quality filtered using the FASTX toolkit (42) with a stringency of Q30 over ≥95% of each read. The filtered data were processed using IMGT/High-VQUEST (43) and analyzed using custom R scripts, available from the corresponding author by request. Circos analyses were performed using Circos Table Viewer (44).

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Specifically, the sequences associated with each accession number provided herein including for example accession numbers for proteins and/or nucleic acid molecules provided in the Tables or elsewhere, are incorporated by reference in their entirely.

The scope of the claims should not be limited by the preferred embodiments and examples but should be given the broadest interpretation consistent with the description as a whole.

### REFERENCES

1. Bechtel, T. J., and Weerapana, E. (2017) From structure to redox: the diverse functional roles of disulfides and implications in disease. Proteomics. 17, 10.1002/pmic.201600391.
2. Liu, H., and May, K. (2012) Disulfide bond structures of IgG molecules: Structural variations, chemical modifications and possible impacts to stability and biological function. MAbs. 4, 17-23.
3. Dombkowski, A. A., Sultana, K. Z., and Craig, D. B. (2013) Protein disulfide engineering. FEBS Lett. 588, 206-212.
4. Willuda, J., Honegger, A., Waibel, R., Schubiger, P. A., Stahel, R., Zangemeister-Wittke, U., and Pluckthun, A. (1999) Thermal stability is essential for tumor targeting of antibody fragments: Engineering of a humanized anti-epithelial glycoprotein-2 (epithelial cell adhesion molecule) single-chain Fv fragment. Cancer Res. 59, 5758-5767.
5. Gong, R., Vu, B. K., Feng, Y., Prieto, D. A., Dyba, M. A., Walsh, J. D., Prabakaran, P., Veenstra, T. D., Tarasov, S. G., Ishima, R., and Dimitrov, D. S. (2009) Engineered human antibody constant domains with increased stability. J. Biol. Chem. 284, 14203-1410.
6. Hagihara, Y., Mine, S., and Uegaki, K. (2007) Stabilization of an immunoglobulin fold domain by an engineered disulfide bond at the buried hydrophobic region. J. Biol. Chem. 282, 36489-36495.
7. Saerens, D., Conrath, K., Govaert, J., and Muyldermans, S. (2008) Disulfide bond introduction for general stabilization of immunoglobulin heavy-chain variable domains. J. Mol. Biol. 377, 478-488.
8. Pan, R., Sampson, J. M., Chen, Y., Vaine, M., Wang, S., Lu, S., and Kong, X. P. (2013) Rabbit anti-HIV-1 monoclonal antibodies raised by immunization can mimic the antigen-binding modes of antibodies derived from HIV-1-infected humans. J. Virol. 87, 10221-10231.
9. Kim, D. Y., Kandalaft, H., Hussack, G., Raphael, S., Ding, W., Kelly, J. F., Henry, K. A., and Tanha, J. (2019) Evaluation of a noncanonical Cys40-Cys55 disulfide linkage for stabilization of single-domain antibodies. Protein Sci. 28, 881-888.
10. de los Rios, M., Criscitiello, M. F., and Smider, V. V. (2015) Structural and genetic diversity in antibody repertoires from diverse species. Curr. Opin. Struct. Biol. 33, 27-41.
11. McConnell, A. D., Spasojevich, V., Macomber, J. L., Kapf, I. P., Chen, A., Sheffer, J. C., Berkebile, A., Horlick, R. A., Neben, S., King, D. J., and Bowers, P. M. (2013) An integrated approach to extreme thermostabilization and affinity maturation of antibody. Protein Eng. Des. Sel. 26, 151-164.
12. McConnell, A. D., Zhang, X., Macomber, J. L., Chau, B., Sheffer, J. C., Rahmanian, S., Hare, E., Spasojevic, V., Horlick, R. A., King, D. J., and Bowers, P. M. (2014) A general approach to antibody thermostabilization. MAbs. 6, 1274-1282.
13. Wozniak-Knopp, G., Stadlmann, J., and Rüker, F. (2012) Stabilisation of the Fc fragment of human IgG1 by engineered intradomain disulfide bonds. PLoS One. 7, e30083.
14. Zabetakis, D., Olson, M. A., Anderson, G. P., Legler, P. M., and Goldman, E. R. (2014) Evaluation of disulfide bond position to enhance the thermal stability of a highly stable single domain antibody. PLoS One. 9, e115405.
15. Wang, F., Ekiert, D. C., Ahmad, I., Yu, W., Zhang, Y., Bazirgan, O., Torkamani, A., Raudsepp, T., Mwangi, W., Criscitiello, M. F., Wilson, I. A., Schultz, P. G. and Smider, V. V. (2013) Reshaping antibody diversity. Cell. 153, 1379-1393.
16. Prabakaran, P., and Chowdhury, P. S. (2020) Landscape of non-canonical cysteines in human VH repertoire revealed by immunogenetic analysis. Cell Rep. 31, 107831.
17. Graille, M., Stura, E. A., Housden, N. G., Beckingham, J. A., Bottomley, S. P., Beale, D., Taussig, M. J., Sutton, B. J., Gore, M. G., and Charbonnier, J. B. (2001) Complex between Peptostreptococcus magnus protein L and a human antibody reveals structural convergence in the interaction modes of Fab binding proteins. Structure. 9, 679-687.
18. Graille, M., Stura, E. A., Corper, A. L., Sutton, B. J., Taussig, M. J., Charbonnier, J. B., and Silverman, G. J. (2000) Crystal structure of a Staphylococcus aureus protein A domain complexed with the Fab fragment of a human IgM antibody: Structural basis for recognition of B-cell receptors and superantigen activity. Proc. Natl. Acad. Sci. USA. 97, 5399-5404.
19. Henry, K. A., Kim, D. Y., Kandalaft, H., Lowden, M. J., Yang, Q., Schrag, J. D., Hussack G., MacKenzie, C. R., and Tanha, J. (2017) Stability-diversity tradeoffs impose fundamental constraints on selection of synthetic human VH/VL single-domain antibodies from in vitro display libraries. Front. Immunol. 8, 1759.
20. To, R., Hirama, T., Arbabi-Ghahroudi, M., MacKenzie, R., Wang, P., Xu, P., Ni, F., and Tanha, J. (2005) Isolation of monomeric human VHs by a phage selection. J. Biol. Chem. 280, 41395-41403.
21. Hussack, G., Arbabi-Ghahroudi, M., van Faassen, H., Songer, J. G., Ng, K. K., MacKenzie, R., and Tanha, J. (2011) Neutralization of Clostridium difficile toxin A with single-domain antibodies targeting the cell receptor binding domain. J. Biol. Chem. 286, 8961-8976.
22. Kim, D. Y., To, R., Kandalaft, H., Ding, W., van Faassen, H., Luo, Y., Schrag, J. D., St-Amant, N., Hefford, M., Hirama, T., Kelly, J. F., MacKenzie, R., and Tanha, J. (2014) Antibody light chain variable domains and their biophysically improved versions for human immunotherapy. MAbs. 6, 219-235.
23. Hussack, G., Ryan, S., van Faassen, H., Rossotti, M., MacKenzie, C. R., and Tanha, J. (2018) Neutralization of Clostridium difficile toxin B with VHH-Fc fusions targeting the delivery and CROPs domains. PLoS One. 13, e0208978.
24. Bell, A., Wang, Z. J., Arbabi-Ghahroudi, M., Chang, T. A., Durocher, Y., Trojahn, U., Baardsnes, J., Jaramillo, M. J., Li, S., Baral, T. N., O'Connor-McCourt, M., MacKenzie, R., and Zhang, J. (2010) Differential tumor-targeting abilities of three single-domain antibody formats. Cancer Lett. 289: 81-90.
25. Stanimirovic, D., Kemmerich, K., Haqqani, A. S., Sulea, T., Arbabi-Ghahroudi, M., Massie B., and Gilbert, R., inventors; National Research Council Canada, assignee (2015) Insulin-like growth factor 1 receptor-specific antibodies and uses thereof. United States patent US 10100117 B2.
26. Kunz, P., Flock, T., Soler, N., Zaiss, M., Vincke, C., Sterckx, Y., Kastelic, D., Muyldermans, S., and Hoheisel, J. D. (2017) Exploiting sequence and stability information for directing nanobody stability engineering. Biochim. Biophys. Acta Gen. Subj. 1861, 2196-2205.
27. Hussack, G., Hirama, T., Ding, W., MacKenzie, R., and Tanha, J. (2011) Engineered single-domain antibodies with high protease resistance and thermal stability. PLoS One. 6, e28218.
28. Prabakaran, P., and Chowdhury, P. S. (2020) Landscape of non-canonical cysteines in human VH repertoire revealed by immunogenetic analysis. Cell Rep. 31, 107831.
29. Hagihara, Y., and Saerens, D. (2014) Engineering disulfide bonds within an antibody. Biochim. Biophys. Acta. 1844, 2016-2023.
30. Haakenson, J. K., Deiss, T. C., Warner, G. F., Mwangi, W., Criscitiello, M. F., and Smider, V. V. (2019) A broad role for cysteines in bovine antibody diversity. ImmunoHorizons. 3, 478-487.
31. Govaert, J., Pellis, M., Deschacht, N., Vincke, C., Conrath, K, Muyldermans, S., and Saerens, D. (2012) Dual beneficial effect of interloop disulfide bond for single domain antibody fragments. J. Biol. Chem. 287, 1970-1979.
32. Mendoza, M. N., Jian, M, King, M. T., and Brooks, C. L. (2020) Role of a noncanonical disulfide bond in the stability, affinity, and flexibility of a VHH specific for the Listeria virulence factor InIB. Protein Sci. 29, 990-1003.
33. MacKenzie, C. R., and To, R. (1998) The role of valency in the selection of anti-carbohydrate single-chain Fvs from phage display libraries. J. Immunol. Methods. 220, 39-49.
34. Henry, K. A., Kandalaft, H., Lowden, M. J., Rossotti, M. A., van Faassen, H., Hussack, G., Durocher, Y., Kim, D. Y., and Tanha, J. (2017) A disulfide-stabilized human VL single-domain antibody library is a source of soluble and highly thermostable binders. Mol. Immunol. 90, 190-196.
35. Rajan, S., and Sidhu, S. S. (2012) Simplified synthetic antibody libraries. Methods Enzymol. 502, 3-23.
36. Wickner, W. (1975) Asymmetric orientation of a phage coat protein in cytoplasmic membrane of Escherichia coli. Proc. Natl. Acad. Sci. USA. 72, 4749-4753.
37. Baral, T. N., MacKenzie, R., and Arbabi-Ghahroudi, M. (2013) Single-domain antibodies and their utility. Curr. Protoc. Immunol. 103, Unit 2.17.
38. Robotham, A. C., and Kelley, J. F. (2019) Detection and quantification of free sulfhydryls in monoclonal antibodies using maleimide labeling and mass spectrometry. MAbs. 11, 757-766.
39. Henry, K. A. (2018) Next-generation DNA sequencing of VH/VL repertoires: A primer and guide to applications in single-domain antibody discovery. Methods Mol. Biol. 1701, 425-446.
40. Henry, K. A. (2013) HIV-associated modulation of serological and immunogenetic features of humoral immunity. Ph.D. thesis, Simon Fraser University.
41. Magoc, T, and Salzberg, S. L. (2011) FLASH: fast length adjustment of short reads to improve genome assemblies. Bioinformatics. 27, 2957-2963.
42. Schmieder, R., and Edwards, R. (2011) Quality control and preprocessing of metagenomic datasets. Bioinformatics. 27, 863-864.
43. Alamyar, E., Duroux, P., Lefranc, M. P., Giudicelli, V. (2012) IMGT@tools forthe nucleotide analysis of immunoglobulin (IG) and T cell receptor (TR) V-(D)-J repertoires, polymorphisms, and IG mutations: IMGTN-QUEST and IMGT/HighV-QUEST for NGS. Methods Mol. Biol. 882, 569-604.
44. Krzywinski, M. I., Schein, J. E., Birol, I., Connors, J., Gascoyne, R., Horsman, D., Jones, S. J., and Marra, M. A. (2009) Circos: An information aesthetic for comparative genomics. 19, 1639-1645.
45. Lefranc, M-P., Pommie, C., Ruiz, M., Giudicelli, V., Foulquier, E., Truong, L., Thouvenin-Contet, V., Lefranc, G. (2003) IMGT unique numbering for immunoglobulin and T cell receptor variable domains and Ig superfamily V-like domains, Developmental & Comparative Immunology, 27(1), 55-77.

1. An engineered immunoglobulin (Ig) variable domain comprising an introduced pair of cysteine residues comprising a first cysteine residue and a second cysteine residue, wherein:
the first cysteine residue is at position 24 and the second cysteine residue is at position 86;
the first cysteine residue is at position 22 and the second cysteine residue is at position 88;
the first cysteine residue is at position 4 and the second cysteine residue is at position 25;
the first cysteine residue is at position 4 and the second cysteine residue is at position 118;
the first cysteine residue is at position 5 and the second cysteine residue is at position 120;
the first cysteine residue is at position 6 and the second cysteine residue is at position 119;
or
the first cysteine residue is at position 45 and the second cysteine residue is at position 100,
wherein the position numbering refers to IMGT numbering and wherein at least one disulfide bond is formed or is capable of forming between the first and second cysteine residues.

2. The engineered Ig variable domain of claim 1, wherein the variable domain is a heavy chain variable domain, a V_{H}H variable domain, a light chain variable domain, or a V_{NAR} variable domain.

3. (canceled)

4. The engineered Ig variable domain of claim 1, wherein the engineered Ig variable domain comprises any one of the amino acid sequences set forth in SEQ ID NOs: 332-338, 340-346, 348-354, 356-362, 364-370, 372-378, 380-386, 388-394, 396-402, 404-410, 412-418, 420-426, 428-434, 436-442, 444-450, 452-458, 460-466, 468-474, 476-482, 484-490, 492-498, 500-506, 508-514, 516-522, 524-530, 532-538, 540-546, 548-554, 556-562, 564-570, 572-578, 580-586, 588-594, and 596-602i or an amino acid sequence having at least 80% sequence identity to any one of the amino acid sequences set forth in SEQ ID NOs: 332-338, 340-346, 348-354, 356-362, 364-370, 372-378, 380-386, 388-394, 396-402, 404-410, 412-418, 420-426, 428-434, 436-442, 444-450, 452-458, 460-466, 468-474, 476-482, 484-490, 492-498, 500-506, 508-514, 516-522, 524-530, 532-538, 540-546, 548-554, 556-562, 564-570, 572-578, 580-586, 588-594, and 596-602, wherein the first and second cysteine residues are maintained.

5. (canceled)

6. An antibody or antigen-binding fragment thereof comprising at least one engineered Ig variable domain of claim 1.

7. The antibody or antigen-binding fragment thereof of claim 6, which is:
a single domain antibody;
an IgG1, IgG2, IgG3, IgG4, IgE, IgA, IgY, IgD, IgM, and/or IgNAR antibody; or
an antibody fragment selected from Fab, Fab′, F(ab′)2, scFv, dsFv, and ds-scFv: or a dimer, minibody, diabody, or multimer thereof.

8. (canceled)

9. (canceled)

10. A polypeptide comprising more than one engineered Ig variable domain of claim 1.

11. A nucleic acid molecule encoding the engineered Ig variable domain of claim 1.

12. (canceled)

13. (canceled)

14. (canceled)

15. A cell or animal expressing the engineered Ig variable domain of claim 1.

16. (canceled)

17. A composition comprising the engineered Ig variable domain of claim 1 and a pharmaceutically acceptable carrier and/or excipient.

18. (canceled)

19. An engineered library comprising at least one nucleic acid molecule encoding at least one engineered Ig variable domain of claim 1.

20. The engineered library of claim 19, wherein the at least one engineered Ig variable domain is a plurality and wherein at least a subset of the plurality comprises different CDR sequences.

21. A method of making an antibody or antigen-binding fragment thereof with increased stability relative to a control, the method comprising generating an antibody or antigen-binding fragment thereof comprising at least one engineered Ig variable domain of claim 1, wherein the control is an antibody or antigen-binding fragment thereof into which the pair of cysteine residues has not been introduced but is otherwise the same as the antibody or antigen-binding fragment thereof produced by the method.

22. A method of increasing stability of an antibody or antigen-binding fragment thereof relative to a control, the method comprising introducing into at least one variable domain of the antibody or antigen-binding fragment thereof a pair of cysteine residues, wherein the pair of cysteine residues is selected from:
a cysteine residue at position 24 and a cysteine residue at position 86;
a cysteine residue at position 22 and a cysteine residue at position 88;
a cysteine residue at position 4 and a cysteine residue at position 25;
a cysteine residue at position 4 and a cysteine residue at position 118;
a cysteine residue at position 5 and a cysteine residue at position 120;
a cysteine residue at position 6 and a cysteine residue at position 119; and
a cysteine residue at position 45 and a cysteine residue at position 100,
wherein:
the position numbering refers to IMGT numbering,
the cysteine residues introduced form or are capable of forming a disulfide bond, and
the control is an antibody or antigen-binding fragment thereof into which the pair of cysteine residues has not been introduced but is otherwise the same as the antibody or antigen-binding fragment thereof produced by the method.

23. The method of claim 22, wherein the variable domain is a heavy chain variable domain, a V_{H}H variable domain, a light chain variable domain, or a V_{NAR} variable domain.

24. (canceled)

25. The method of claim 22, wherein the engineered Ig variable domain comprises any one of the amino acid sequences of SEQ ID NOs: 332-338, 340-346, 348-354, 356-362, 364-370, 372-378, 380-386, 388-394, 396-402, 404-410, 412-418, 420-426, 428-434, 436-442, 444-450, 452-458, 460-466, 468-474, 476-482, 484-490, 492-498, 500-506, 508-514, 516-522, 524-530, 532-538, 540-546, 548-554, 556-562, 564-570, 572-578, 580-586, 588-594, and 596-602; or an amino acid sequence having at least 80% sequence identity to any one of the amino acid sequences of SEQ ID NOs: 332-338, 340-346, 348-354, 356-362, 364-370, 372-378, 380-386, 388-394, 396-402, 404-410, 412-418, 420-426, 428-434, 436-442, 444-450, 452-458, 460-466, 468-474, 476-482, 484-490, 492-498, 500-506, 508-514, 516-522, 524-530, 532-538, 540-546, 548-554, 556-562, 564-570, 572-578, 580-586, 588-594, and 596-602, wherein the one or more than one cysteine pair is maintained.

26. The method of claim 21, wherein the antibody or antigen-binding fragment thereof has increased thermostability and/or increased resistance to protease activity relative to the control.

27. A method of identifying one or more than one location in an engineered immunoglobulin (Ig) variable domain tolerant of disulfide bonds, the method comprising:
a) generating a library of nucleic acid molecules encoding an engineered immunoglobulin (Ig) variable domain lacking a cysteine residue at position 23 and/or lacking a cysteine residue at position 104 and thus lacking a disulfide bond between position 23 and position 104, wherein at least one cysteine residue has been introduced into the amino acid sequence of each engineered Ig variable domain encoded by the nucleic acid molecules of the library;
b) identifying Ig variable domains encoded by the nucleic acid molecules of the library which have formed one or more than one disulfide bond other than between positions 23 and 104; and
c) identifying the positions between which the one or more than one disulfide bond is formed in each engineered Ig variable domain,
wherein the position numbering refers to IMGT numbering.

28. A method of identifying engineered immunoglobulin (Ig) variable domain disulfide bonds that increase stability, the method comprising:
a) generating a library of nucleic acid molecules encoding engineered immunoglobulin (Ig) variable domains lacking a cysteine residue at position 23 and/or lacking a cysteine residue at position 104 and thus lacking a disulfide bond between position 23 and position 104, wherein engineered pairs of cysteine residues have been introduced into the amino acid sequence of each engineered Ig variable domain encoded by the nucleic acid molecules of the library;
b) identifying Ig variable domains encoded by the nucleic acid molecules of the library which have formed at least one disulfide bond other than between positions 23 and 104;
c) determining the stability of the identified Ig variable domains, and
d) identifying the positions between which the disulfide bonds are formed in each engineered Ig variable domain that has increased stability as compared to a control,
wherein the position numbering refers to IMGT numbering.

29. (canceled)

30. (canceled)

31. (canceled)

32. (canceled)

33. The method of claim 28, wherein the stability is determined by measuring thermostability and/or protease resistance of the engineered Ig variable domain.

34. (canceled)

35. (canceled)

36. The engineered library of claim 19, wherein the at least one nucleic acid molecule is a plurality, and wherein each of the nucleic acid molecules encodes an engineered Ig variable domain or an antibody or antigen-binding fragment thereof, wherein the Ig variable domains or the antibodies or antigen-binding fragments thereof encoded by the nucleic acid molecules comprise the same engineered cysteine residues but have different framework and/or CDR sequences.


SEQUENCE LISTING (615 sequences)

SEQ ID NO: 1 - DNA, 31 bp, synthetic construct
  source 1..31: mol_type = other DNA
caccagctggacgcatgcactgtgagaatag

SEQ ID NO: 2 - DNA, 27 bp, synthetic construct
  source 1..27: mol_type = other DNA
ctgcaccagctggcactgtgcactgtg

SEQ ID NO: 3 - DNA, 28 bp, synthetic construct
  source 1..28: mol_type = other DNA
agactgcaccaggcagacctgtgcactg

SEQ ID NO: 4 - DNA, 27 bp, synthetic construct
  source 1..27: mol_type = other DNA
cccagactgcacgcactggacctgtgc

SEQ ID NO: 5 - DNA, 24 bp, synthetic construct
  source 1..24: mol_type = other DNA
cccagactggcacagctggacctg

SEQ ID NO: 6 - DNA, 27 bp, synthetic construct
  source 1..27: mol_type = other DNA
gcctcccccagagcacaccagctggac

SEQ ID NO: 7 - DNA, 27 bp, synthetic construct
  source 1..27: mol_type = other DNA
caagcctcccccgcactgcaccagctg

SEQ ID NO: 8 - DNA, 27 bp, synthetic construct
  source 1..27: mol_type = other DNA
gaccaagcctccgcaagactgcaccag

SEQ ID NO: 9 - DNA, 27 bp, synthetic construct
  source 1..27: mol_type = other DNA
cttgaccaagccgcacccagactgcac

SEQ ID NO: 10 - DNA, 27 bp, synthetic construct
  source 1..27: mol_type = other DNA
aggcttgaccaagcatcccccagactg

SEQ ID NO: 11 - DNA, 28 bp, synthetic construct
  source 1..28: mol_type = other DNA
tccaggcttgacgcagcctcccccagac

SEQ ID NO: 12 - DNA, 27 bp, synthetic construct
  source 1..27: mol_type = other DNA
ccctccaggcttgcacaagcctccccc

SEQ ID NO: 13 - DNA, 27 bp, synthetic construct
  source 1..27: mol_type = other DNA
ggaccctccagggcagaccaagcctcc

SEQ ID NO: 14 - DNA, 27 bp, synthetic construct
  source 1..27: mol_type = other DNA
cagggaccctccgcacttgaccaagcc

SEQ ID NO: 15 - DNA, 32 bp, synthetic construct
  source 1..32: mol_type = other DNA
agtctcagggacccgcaaggcttgaccaagcc

SEQ ID NO: 16 - DNA, 33 bp, synthetic construct
  source 1..33: mol_type = other DNA
gagagtctcagggagcatccaggcttgaccaag

SEQ ID NO: 17 - DNA, 33 bp, synthetic construct
  source 1..33: mol_type = other DNA
gcggagagtctcaggcaccctccaggcttgacc

SEQ ID NO: 18 - DNA, 33 bp, synthetic construct
  source 1..33: mol_type = other DNA
gctgcggagagtctgcaggaccctccaggcttg

SEQ ID NO: 19 - DNA, 33 bp, synthetic construct
  source 1..33: mol_type = other DNA
gctgctgcggagaggcacagggaccctccaggc

SEQ ID NO: 20 - DNA, 32 bp, synthetic construct
  source 1..32: mol_type = other DNA
gaggctgctgcggagcatctcagggaccctcc

SEQ ID NO: 21 - DNA, 32 bp, synthetic construct
  source 1..32: mol_type = other DNA
ccagaggctgctgcgcagagtctcagggaccc

SEQ ID NO: 22 - DNA, 33 bp, synthetic construct
  source 1..33: mol_type = other DNA
aatccagaggctgcgcaggagagtctcagggac

SEQ ID NO: 23 - DNA, 33 bp, synthetic construct
  source 1..33: mol_type = other DNA
gtgaatccagaggcgcatgcggagagtctcagg

SEQ ID NO: 24 - DNA, 33 bp, synthetic construct
  source 1..33: mol_type = other DNA
aaggtgaatccagagcatgctgcggagagtctc

SEQ ID NO: 25 - DNA, 34 bp, synthetic construct
  source 1..34: mol_type = other DNA
ctgaaggtgaatccgcaggctgctgcggagagtc

SEQ ID NO: 26 - DNA, 28 bp, synthetic construct
  source 1..28: mol_type = other DNA
gcggacccagctgcagtagtagtcactg

SEQ ID NO: 27 - DNA, 27 bp, synthetic construct
  source 1..27: mol_type = other DNA
ctggcggacccagcacatgtagtagtc

SEQ ID NO: 28 - DNA, 28 bp, synthetic construct
  source 1..28: mol_type = other DNA
agcctggcggacgcagctcatgtagtag

SEQ ID NO: 29 - DNA, 28 bp, synthetic construct
  source 1..28: mol_type = other DNA
tggagcctggcggcaccagctcatgtag

SEQ ID NO: 30 - DNA, 28 bp, synthetic construct
  source 1..28: mol_type = other DNA
ccctggagcctggcagacccagctcatg

SEQ ID NO: 31 - DNA, 28 bp, synthetic construct
  source 1..28: mol_type = other DNA
cttccctggagcgcagcggacccagctc

SEQ ID NO: 32 - DNA, 28 bp, synthetic construct
  source 1..28: mol_type = other DNA
tcccttccctgggcactggcggacccag

SEQ ID NO: 33 - DNA, 27 bp, synthetic construct
  source 1..27: mol_type = other DNA
cagtcccttcccgcaagcctggcggac

SEQ ID NO: 34 - skipped

SEQ ID NO: 35 - DNA, 29 bp, synthetic construct
  source 1..29: mol_type = other DNA
ctggcggacccagcacatgtagtagtcac

SEQ ID NO: 36-39 - skipped (4 entries)

SEQ ID NO: 40 - DNA, 27 bp, synthetic construct
  source 1..27: mol_type = other DNA
ctccagtcccttgcatggagcctggcg

SEQ ID NO: 41 - DNA, 27 bp, synthetic construct
  source 1..27: mol_type = other DNA
ccactccagtccgcaccctggagcctg

SEQ ID NO: 42 - DNA, 27 bp, synthetic construct
  source 1..27: mol_type = other DNA
gacccactccaggcacttccctggagc

SEQ ID NO: 43 - DNA, 27 bp, synthetic construct
  source 1..27: mol_type = other DNA
tgagacccactcgcatcccttccctgg

SEQ ID NO: 44 - DNA, 27 bp, synthetic construct
  source 1..27: mol_type = other DNA
aaatgagacccagcacagtcccttccc

SEQ ID NO: 45 - DNA, 28 bp, synthetic construct
  source 1..28: mol_type = other DNA
aataaatgagacgcactccagtcccttc

SEQ ID NO: 46 - DNA, 27 bp, synthetic construct
  source 1..27: mol_type = other DNA
ataaataaatgagcaccactccagtcc

SEQ ID NO: 47 - DNA, 27 bp, synthetic construct
  source 1..27: mol_type = other DNA
gctataaataaagcagacccactccag

SEQ ID NO: 48 - DNA, 27 bp, synthetic construct
  source 1..27: mol_type = other DNA
accgctataaatgcatgagacccactc

SEQ ID NO: 49 - DNA, 27 bp, synthetic construct
  source 1..27: mol_type = other DNA
ggagtctgcgtagcatgtgctaccacc

SEQ ID NO: 50 - DNA, 27 bp, synthetic construct
  source 1..27: mol_type = other DNA
cacggagtctgcgcagtatgtgctacc

SEQ ID NO: 51 - DNA, 29 bp, synthetic construct
  source 1..29: mol_type = other DNA
cttcacggagtcgcagtagtatgtgctac

SEQ ID NO: 52 - DNA, 28 bp, synthetic construct
  source 1..28: mol_type = other DNA
gcccttcacggagcatgcgtagtatgtg

SEQ ID NO: 53 - DNA, 29 bp, synthetic construct
  source 1..29: mol_type = other DNA
tcggcccttcacgcagtctgcgtagtatg

SEQ ID NO: 54 - DNA, 28 bp, synthetic construct
  source 1..28: mol_type = other DNA
gaatcggcccttgcaggagtctgcgtag

SEQ ID NO: 55 - DNA, 27 bp, synthetic construct
  source 1..27: mol_type = other DNA
ggtgaatcggccgcacacggagtctgc

SEQ ID NO: 56 - DNA, 27 bp, synthetic construct
  source 1..27: mol_type = other DNA
gatggtgaatcggcacttcacggagtc

SEQ ID NO: 57 - DNA, 28 bp, synthetic construct
  source 1..28: mol_type = other DNA
ggagatggtgaagcagcccttcacggag

SEQ ID NO: 58 - DNA, 27 bp, synthetic construct
  source 1..27: mol_type = other DNA
tctggagatggtgcatcggcccttcac

SEQ ID NO: 59 - DNA, 28 bp, synthetic construct
  source 1..28: mol_type = other DNA
gtctctggagatgcagaatcggcccttc

SEQ ID NO: 60 - DNA, 27 bp, synthetic construct
  source 1..27: mol_type = other DNA
attgtctctggagcaggtgaatcggcc

SEQ ID NO: 61 - DNA, 27 bp, synthetic construct
  source 1..27: mol_type = other DNA
ggaattgtctctgcagatggtgaatcg

SEQ ID NO: 62 - DNA, 29 bp, synthetic construct
  source 1..29: mol_type = other DNA
cttggaattgtcgcaggagatggtgaatc

SEQ ID NO: 63 - DNA, 28 bp, synthetic construct
  source 1..28: mol_type = other DNA
gttcttggaattgcatctggagatggtg

SEQ ID NO: 64 - DNA, 28 bp, synthetic construct
  source 1..28: mol_type = other DNA
cgtgttcttggagcagtctctggagatg

SEQ ID NO: 65 - DNA, 28 bp, synthetic construct
  source 1..28: mol_type = other DNA
cagcgtgttcttgcaattgtctctggag

SEQ ID NO: 66 - DNA, 28 bp, synthetic construct
  source 1..28: mol_type = other DNA
atacagcgtgttgcaggaattgtctctg

SEQ ID NO: 67 - DNA, 27 bp, synthetic construct
  source 1..27: mol_type = other DNA
aagatacagcgtgcacttggaattgtc

SEQ ID NO: 68 - DNA, 28 bp, synthetic construct
  source 1..28: mol_type = other DNA
ttgaagatacaggcagttcttggaattg

SEQ ID NO: 69 - DNA, 31 bp, synthetic construct
  source 1..31: mol_type = other DNA
catttgaagatagcacgtgttcttggaattg

SEQ ID NO: 70 - DNA, 28 bp, synthetic construct
  source 1..28: mol_type = other DNA
gttcatttgaaggcacagcgtgttcttg

SEQ ID NO: 71 - DNA, 28 bp, synthetic construct
  source 1..28: mol_type = other DNA
gctgttcatttggcaatacagcgtgttc

SEQ ID NO: 72 - DNA, 28 bp, synthetic construct
  source 1..28: mol_type = other DNA
caggctgttcatgcaaagatacagcgtg

SEQ ID NO: 73 - DNA, 27 bp, synthetic construct
  source 1..27: mol_type = other DNA
tctcaggctgttgcattgaagatacag

SEQ ID NO: 74 - DNA, 28 bp, synthetic construct
  source 1..28: mol_type = other DNA
agctctcaggctgcacatttgaagatac

SEQ ID NO: 75 - DNA, 27 bp, synthetic construct
  source 1..27: mol_type = other DNA
ctcagctctcaggcagttcatttgaag

SEQ ID NO: 76 - DNA, 27 bp, synthetic construct
  source 1..27: mol_type = other DNA
gtcctcagctctgcagctgttcatttg

SEQ ID NO: 77 - DNA, 30 bp, synthetic construct
  source 1..30: mol_type = other DNA
cgtgtcctcagcgcacaggctgttcatttg

SEQ ID NO: 78 - DNA, 28 bp, synthetic construct
  source 1..28: mol_type = other DNA
agccgtgtcctcgcatctcaggctgttc

SEQ ID NO: 79 - DNA, 34 bp, synthetic construct
  source 1..34: mol_type = other DNA
atacacagccgtgtcgcaagctctcaggctgttc

SEQ ID NO: 80 - DNA, 33 bp, synthetic construct
  source 1..33: mol_type = other DNA
taatacacagccgtgcactcagctctcaggctg

SEQ ID NO: 81 - DNA, 35 bp, synthetic construct
  source 1..35: mol_type = other DNA
tgcgtaatacacagcgcagtcctcagctctcaggc

SEQ ID NO: 82 - DNA, 32 bp, synthetic construct
  source 1..32: mol_type = other DNA
cgctgcgtaatacacgcacgtgtcctcagctc

SEQ ID NO: 83 - DNA, 33 bp, synthetic construct
  source 1..33: mol_type = other DNA
tctcgctgcgtaatagcaagccgtgtcctcagc

SEQ ID NO: 84 - DNA, 33 bp, synthetic construct
  source 1..33: mol_type = other DNA
ctctctcgctgcgtagcacacagccgtgtcctc

SEQ ID NO: 85 - DNA, 33 bp, synthetic construct
  source 1..33: mol_type = other DNA
gctctctctcgctgcgcaatacacagccgtgtc

SEQ ID NO: 86 - DNA, 32 bp, synthetic construct
  source 1..32: mol_type = other DNA
tctgctctctctcgcgcagtaatacacagccg

SEQ ID NO: 87 - DNA, 27 bp, synthetic construct
  source 1..27: mol_type = other DNA
tgtcccttggccgcagatatcaaaagc

SEQ ID NO: 88 - DNA, 30 bp, synthetic construct
  source 1..30: mol_type = other DNA
cattgtcccttggcaccagatatcaaaagc

SEQ ID NO: 89 - DNA, 27 bp, synthetic construct
  source 1..27: mol_type = other DNA
gaccattgtcccgcagccccagatatc

SEQ ID NO: 90 - DNA, 30 bp, synthetic construct
  source 1..30: mol_type = other DNA
ggtgaccattgtgcattggccccagatatc

SEQ ID NO: 91 - DNA, 28 bp, synthetic construct
  source 1..28: mol_type = other DNA
gacggtgaccatgcacccttggccccag

SEQ ID NO: 92 - DNA, 27 bp, synthetic construct
  source 1..27: mol_type = other DNA
agagacggtgacgcatgtcccttggcc

SEQ ID NO: 93 - DNA, 27 bp, synthetic construct
  source 1..27: mol_type = other DNA
tgaagagacggtgcacattgtcccttg

SEQ ID NO: 94 - DNA, 27 bp, synthetic construct
  source 1..27: mol_type = other DNA
gcctgaagagacgcagaccattgtccc

SEQ ID NO: 95 - DNA, 28 bp, synthetic construct
  source 1..28: mol_type = other DNA
agagcctgaagagcaggtgaccattgtc

SEQ ID NO: 96 - DNA, 29 bp, synthetic construct
  source 1..29: mol_type = other DNA
accagagcctgagcagacggtgaccattg

SEQ ID NO: 97 - DNA, 27 bp, synthetic construct
  source 1..27: mol_type = other DNA
ggaaccagagccgcaagagacggtgac

SEQ ID NO: 98 - DNA, 54 bp, synthetic construct
  source 1..54: mol_type = other DNA
tctcagggacccgcaaggcttgaccaagcctcccccagagcacaccagctggac

SEQ ID NO: 99 - DNA, 52 bp, synthetic construct
  source 1..52: mol_type = other DNA
tctcagggacccgcaaggcttgaccaagcctcccccgcactgcaccagctgg

SEQ ID NO: 100 - DNA, 54 bp, synthetic construct
  source 1..54: mol_type = other DNA
gagtctcagggagcatccaggcttgaccaagcctcccccgcactgcaccagctg

SEQ ID NO: 101 - DNA, 48 bp, synthetic construct
  source 1..48: mol_type = other DNA
tctcagggacccgcaaggcttgaccaagcctccgcaagactgcaccag

SEQ ID NO: 102 - DNA, 51 bp, synthetic construct
  source 1..51: mol_type = other DNA
gagtctcagggagcatccaggcttgaccaagcctccgcaagactgcaccag

SEQ ID NO: 103 - DNA, 54 bp, synthetic construct
  source 1..54: mol_type = other DNA
ggagagtctcaggcaccctccaggcttgaccaagcctccgcaagactgcaccag

SEQ ID NO: 104 - DNA, 45 bp, synthetic construct
  source 1..45: mol_type = other DNA
tctcagggacccgcaaggcttgaccaagccgcacccagactgcac

SEQ ID NO: 105 - DNA, 48 bp, synthetic construct
  source 1..48: mol_type = other DNA
gagtctcagggagcatccaggcttgaccaagccgcacccagactgcac

SEQ ID NO: 106 - DNA, 51 bp, synthetic construct
  source 1..51: mol_type = other DNA
ggagagtctcaggcaccctccaggcttgaccaagccgcacccagactgcac

SEQ ID NO: 107 - DNA, 54 bp, synthetic construct
  source 1..54: mol_type = other DNA
tgcggagagtctgcaggaccctccaggcttgaccaagccgcacccagactgcac

SEQ ID NO: 108 - DNA, 42 bp, synthetic construct
  source 1..42: mol_type = other DNA
tctcagggacccgcaaggcttgaccaagcatcccccagactg

SEQ ID NO: 109 - DNA, 45 bp, synthetic construct
  source 1..45: mol_type = other DNA
gagtctcagggagcatccaggcttgaccaagcatcccccagactg

SEQ ID NO: 110 - DNA, 48 bp, synthetic construct
  source 1..48: mol_type = other DNA
ggagagtctcaggcaccctccaggcttgaccaagcatcccccagactg

SEQ ID NO: 111 - DNA, 51 bp, synthetic construct
  source 1..51: mol_type = other DNA
tgcggagagtctgcaggaccctccaggcttgaccaagcatcccccagactg

SEQ ID NO: 112 - DNA, 54 bp, synthetic construct
  source 1..54: mol_type = other DNA
tgctgcggagaggcacagggaccctccaggcttgaccaagcatcccccagactg

SEQ ID NO: 113 - DNA, 40 bp, synthetic construct
  source 1..40: mol_type = other DNA
tctcagggacccgcaaggcttgacgcagcctcccccagac

SEQ ID NO: 114 - DNA, 43 bp, synthetic construct
  source 1..43: mol_type = other DNA
gagtctcagggagcatccaggcttgacgcagcctcccccagac

SEQ ID NO: 115 - DNA, 46 bp, synthetic construct
  source 1..46: mol_type = other DNA
ggagagtctcaggcaccctccaggcttgacgcagcctcccccagac

SEQ ID NO: 116 - DNA, 49 bp, synthetic construct
  source 1..49: mol_type = other DNA
tgcggagagtctgcaggaccctccaggcttgacgcagcctcccccagac

SEQ ID NO: 117 - DNA, 52 bp, synthetic construct
  source 1..52: mol_type = other DNA
tgctgcggagaggcacagggaccctccaggcttgacgcagcctcccccagac

SEQ ID NO: 118 - DNA, 55 bp, synthetic construct
  source 1..55: mol_type = other DNA
ggctgctgcggagcatctcagggaccctccaggcttgacgcagcctcccccagac

SEQ ID NO: 119 - DNA, 36 bp, synthetic construct
  source 1..36: mol_type = other DNA
tctcagggacccgcaaggcttgcacaagcctccccc

SEQ ID NO: 120 - DNA, 39 bp, synthetic construct
  source 1..39: mol_type = other DNA
gagtctcagggagcatccaggcttgcacaagcctccccc

SEQ ID NO: 121 - DNA, 42 bp, synthetic construct
  source 1..42: mol_type = other DNA
ggagagtctcaggcaccctccaggcttgcacaagcctccccc

SEQ ID NO: 122 - DNA, 45 bp, synthetic construct
  source 1..45: mol_type = other DNA
tgcggagagtctgcaggaccctccaggcttgcacaagcctccccc

SEQ ID NO: 123 - DNA, 48 bp, synthetic construct
  source 1..48: mol_type = other DNA
tgctgcggagaggcacagggaccctccaggcttgcacaagcctccccc

SEQ ID NO: 124 - DNA, 51 bp, synthetic construct
  source 1..51: mol_type = other DNA
ggctgctgcggagcatctcagggaccctccaggcttgcacaagcctccccc

SEQ ID NO: 125 - DNA, 54 bp, synthetic construct
  source 1..54: mol_type = other DNA
agaggctgctgcgcagagtctcagggaccctccaggcttgcacaagcctccccc

SEQ ID NO: 126 - DNA, 39 bp, synthetic construct
  source 1..39: mol_type = other DNA
gagtctcagggacccgcaagggcagaccaagcctccccc

SEQ ID NO: 127 - DNA, 36 bp, synthetic construct
  source 1..36: mol_type = other DNA
gagtctcagggagcatccagggcagaccaagcctcc

SEQ ID NO: 128 - DNA, 40 bp, synthetic construct
  source 1..40: mol_type = other DNA
cggagagtctcaggcaccctccagggcagaccaagcctcc

SEQ ID NO: 129 - DNA, 42 bp, synthetic construct
  source 1..42: mol_type = other DNA
tgcggagagtctgcaggaccctccagggcagaccaagcctcc

SEQ ID NO: 130 - DNA, 45 bp, synthetic construct
  source 1..45: mol_type = other DNA
tgctgcggagaggcacagggaccctccagggcagaccaagcctcc

SEQ ID NO: 131 - DNA, 48 bp, synthetic construct
  source 1..48: mol_type = other DNA
ggctgctgcggagcatctcagggaccctccagggcagaccaagcctcc

SEQ ID NO: 132 - DNA, 48 bp, synthetic construct
  source 1..48: mol_type = other DNA
ggctgctgcgcagagtctcagggaccctccagggcagaccaagcctcc

SEQ ID NO: 133 - DNA, 54 bp, synthetic construct
  source 1..54: mol_type = other DNA
tccagaggctgcgcaggagagtctcagggaccctccagggcagaccaagcctcc

SEQ ID NO: 134 - DNA, 42 bp, synthetic construct
  source 1..42: mol_type = other DNA
ggagagtctcagggacccgcagcacttgaccaagcctccccc

SEQ ID NO: 135 - DNA, 39 bp, synthetic construct
  source 1..39: mol_type = other DNA
ggagagtctcagggagcatccgcacttgaccaagcctcc

SEQ ID NO: 136 - DNA, 42 bp, synthetic construct
  source 1..42: mol_type = other DNA
ctgcggagagtctcaggcaccctccgcacttgaccaagcctc

SEQ ID NO: 137 - DNA, 40 bp, synthetic construct
  source 1..40: mol_type = other DNA
gcggagagtctgcaggaccctccgcacttgaccaagcctc

SEQ ID NO: 138 - DNA, 42 bp, synthetic construct
  source 1..42: mol_type = other DNA
tgctgcggagaggcacagggaccctccgcacttgaccaagcc

SEQ ID NO: 139 - DNA, 45 bp, synthetic construct
  source 1..45: mol_type = other DNA
ggctgctgcggagcatctcagggaccctccgcacttgaccaagcc

SEQ ID NO: 140 - DNA, 50 bp, synthetic construct
  source 1..50: mol_type = other DNA
agaggctgctgcgcagagtctcagggaccctccgcacttgaccaagcctc

SEQ ID NO: 141 - DNA, 53 bp, synthetic construct
  source 1..53: mol_type = other DNA
tccagaggctgcgcaggagagtctcagggaccctccgcacttgaccaagcctc

SEQ ID NO: 142 - DNA, 56 bp, synthetic construct
  source 1..56: mol_type = other DNA
gaatccagaggcgcatgcggagagtctcagggaccctccgcacttgaccaagcctc

SEQ ID NO: 143 - DNA, 55 bp, synthetic construct
  source 1..55: mol_type = other DNA
gcggacccagctgcagtagtagtcactgaaggtgaatccgcaggctgctgcggag

SEQ ID NO: 144 - DNA, 52 bp, synthetic construct
  source 1..52: mol_type = other DNA
ctccagtcccttgcatggagcctggcggacccagctgcagtagtagtcactg

SEQ ID NO: 145 - DNA, 55 bp, synthetic construct
  source 1..55: mol_type = other DNA
ccactccagtccgcaccctggagcctggcggacccagctgcagtagtagtcactg

SEQ ID NO: 146 - DNA, 58 bp, synthetic construct
  source 1..58: mol_type = other DNA
gacccactccaggcacttccctggagcctggcggacccagctgcagtagtagtcactg

SEQ ID NO: 147 - DNA, 50 bp, synthetic construct
  source 1..50: mol_type = other DNA
ctccagtcccttgcatggagcctggcggacccagcacatgtagtagtcac

SEQ ID NO: 148 - DNA, 53 bp, synthetic construct
  source 1..53: mol_type = other DNA
ccactccagtccgcaccctggagcctggcggacccagcacatgtagtagtcac

SEQ ID NO: 149 - DNA, 56 bp, synthetic construct
  source 1..56: mol_type = other DNA
gacccactccaggcacttccctggagcctggcggacccagcacatgtagtagtcac

SEQ ID NO: 150 - DNA, 59 bp, synthetic construct
  source 1..59: mol_type = other DNA
tgagacccactcgcatcccttccctggagcctggcggacccagcacatgtagtagtcac

SEQ ID NO: 151 - DNA, 46 bp, synthetic construct
  source 1..46: mol_type = other DNA
ctccagtcccttgcatggagcctggcggacgcagctcatgtagtag

SEQ ID NO: 152 - DNA, 49 bp, synthetic construct
  source 1..49: mol_type = other DNA
ccactccagtccgcaccctggagcctggcggacgcagctcatgtagtag

SEQ ID NO: 153 - DNA, 52 bp, synthetic construct
  source 1..52: mol_type = other DNA
gacccactccaggcacttccctggagcctggcggacgcagctcatgtagtag

SEQ ID NO: 154 - DNA, 55 bp, synthetic construct
  source 1..55: mol_type = other DNA
tgagacccactcgcatcccttccctggagcctggcggacgcagctcatgtagtag

SEQ ID NO: 155 - DNA, 58 bp, synthetic construct
  source 1..58: mol_type = other DNA
aaatgagacccagcacagtcccttccctggagcctggcggacgcagctcatgtagtag

SEQ ID NO: 156 - DNA, 43 bp, synthetic construct
  source 1..43: mol_type = other DNA
ctccagtcccttgcatggagcctggcggcaccagctcatgtag

SEQ ID NO: 157 - DNA, 46 bp, synthetic construct
  source 1..46: mol_type = other DNA
ccactccagtccgcaccctggagcctggcggcaccagctcatgtag

SEQ ID NO: 158 - DNA, 49 bp, synthetic construct
  source 1..49: mol_type = other DNA
gacccactccaggcacttccctggagcctggcggcaccagctcatgtag

SEQ ID NO: 159 - DNA, 52 bp, synthetic construct
  source 1..52: mol_type = other DNA
tgagacccactcgcatcccttccctggagcctggcggcaccagctcatgtag

SEQ ID NO: 160 - DNA, 55 bp, synthetic construct
  source 1..55: mol_type = other DNA
aaatgagacccagcacagtcccttccctggagcctggcggcaccagctcatgtag

SEQ ID NO: 161 - DNA, 58 bp, synthetic construct
  source 1..58: mol_type = other DNA
aataaatgagacgcactccagtcccttccctggagcctggcggcaccagctcatgtag

SEQ ID NO: 162 - DNA, 40 bp, synthetic construct
  source 1..40: mol_type = other DNA
ctccagtcccttgcatggagcctggcagacccagctcatg

SEQ ID NO: 163 - DNA, 43 bp, synthetic construct
  source 1..43: mol_type = other DNA
ccactccagtccgcaccctggagcctggcagacccagctcatg

SEQ ID NO: 164 - DNA, 46 bp, synthetic construct
  source 1..46: mol_type = other DNA
gacccactccaggcacttccctggagcctggcagacccagctcatg

SEQ ID NO: 165 - DNA, 49 bp, synthetic construct
  source 1..49: mol_type = other DNA
tgagacccactcgcatcccttccctggagcctggcagacccagctcatg

SEQ ID NO: 166 - DNA, 52 bp, synthetic construct
  source 1..52: mol_type = other DNA
aaatgagacccagcacagtcccttccctggagcctggcagacccagctcatg

SEQ ID NO: 167 - DNA, 55 bp, synthetic construct
  source 1..55: mol_type = other DNA
aataaatgagacgcactccagtcccttccctggagcctggcagacccagctcatg

SEQ ID NO: 168 - DNA, 58 bp, synthetic construct
  source 1..58: mol_type = other DNA
ataaataaatgagcaccactccagtcccttccctggagcctggcagacccagctcatg

SEQ ID NO: 169 - DNA, 37 bp, synthetic construct
  source 1..37: mol_type = other DNA
ctccagtcccttgcatggagcgcagcggacccagctc

SEQ ID NO: 170 - DNA, 40 bp, synthetic construct
  source 1..40: mol_type = other DNA
ccactccagtccgcaccctggagcgcagcggacccagctc

SEQ ID NO: 171 - DNA, 43 bp, synthetic construct
  source 1..43: mol_type = other DNA
gacccactccaggcacttccctggagcgcagcggacccagctc

SEQ ID NO: 172 - DNA, 46 bp, synthetic construct
  source 1..46: mol_type = other DNA
tgagacccactcgcatcccttccctggagcgcagcggacccagctc

SEQ ID NO: 173 - DNA, 49 bp, synthetic construct
  source 1..49: mol_type = other DNA
aaatgagacccagcacagtcccttccctggagcgcagcggacccagctc

SEQ ID NO: 174 - DNA, 52 bp, synthetic construct
  source 1..52: mol_type = other DNA
aataaatgagacgcactccagtcccttccctggagcgcagcggacccagctc

SEQ ID NO: 175 - DNA, 55 bp, synthetic construct
  source 1..55: mol_type = other DNA
ataaataaatgagcaccactccagtcccttccctggagcgcagcggacccagctc

SEQ ID NO: 176 - DNA, 58 bp, synthetic construct
  source 1..58: mol_type = other DNA
gctataaataaagcagacccactccagtcccttccctggagcgcagcggacccagctc

SEQ ID NO: 177 - DNA, 34 bp, synthetic construct
  source 1..34: mol_type = other DNA
ctccagtcccttgcatgggcactggcggacccag

SEQ ID NO: 178 - DNA, 37 bp, synthetic construct
  source 1..37: mol_type = other DNA
ccactccagtccgcaccctgggcactggcggacccag

SEQ ID NO: 179 - DNA, 40 bp, synthetic construct
  source 1..40: mol_type = other DNA
gacccactccaggcacttccctgggcactggcggacccag

SEQ ID NO: 180 - DNA, 43 bp, synthetic construct
  source 1..43: mol_type = other DNA
tgagacccactcgcatcccttccctgggcactggcggacccag

SEQ ID NO: 181 - DNA, 46 bp, synthetic construct
  source 1..46: mol_type = other DNA
aaatgagacccagcacagtcccttccctgggcactggcggacccag

SEQ ID NO: 182 - DNA, 49 bp, synthetic construct
  source 1..49: mol_type = other DNA
aataaatgagacgcactccagtcccttccctgggcactggcggacccag

SEQ ID NO: 183 - DNA, 52 bp, synthetic construct
  source 1..52: mol_type = other DNA
ataaataaatgagcaccactccagtcccttccctgggcactggcggacccag

SEQ ID NO: 184 - DNA, 55 bp, synthetic construct
  source 1..55: mol_type = other DNA
gctataaataaagcagacccactccagtcccttccctgggcactggcggacccag

SEQ ID NO: 185 - DNA, 58 bp, synthetic construct
  source 1..58: mol_type = other DNA
accgctataaatgcatgagacccactccagtcccttccctgggcactggcggacccag

SEQ ID NO: 186 - DNA, 38 bp, synthetic construct
  source 1..38: mol_type = other DNA
cccactccagtcccttgcagcaagcctggcggacccag

SEQ ID NO: 187 - DNA, 40 bp, synthetic construct
  source 1..40: mol_type = other DNA
gacccactccagtccgcacccgcaagcctggcggacccag

SEQ ID NO: 188 - DNA, 43 bp, synthetic construct
  source 1..43: mol_type = other DNA
tgagacccactccaggcacttcccgcaagcctggcggacccag

SEQ ID NO: 189 - DNA, 39 bp, synthetic construct
  source 1..39: mol_type = other DNA
tgagacccactcgcatcccttcccgcaagcctggcggac

SEQ ID NO: 190 - DNA, 42 bp, synthetic construct
  source 1..42: mol_type = other DNA
aaatgagacccagcacagtcccttcccgcaagcctggcggac

SEQ ID NO: 191 - DNA, 45 bp, synthetic construct
  source 1..45: mol_type = other DNA
aataaatgagacgcactccagtcccttcccgcaagcctggcggac

SEQ ID NO: 192 - DNA, 48 bp, synthetic construct
  source 1..48: mol_type = other DNA
ataaataaatgagcaccactccagtcccttcccgcaagcctggcggac

SEQ ID NO: 193 - DNA, 51 bp, synthetic construct
  source 1..51: mol_type = other DNA
gctataaataaagcagacccactccagtcccttcccgcaagcctggcggac

SEQ ID NO: 194 - DNA, 54 bp, synthetic construct
  source 1..54: mol_type = other DNA
accgctataaatgcatgagacccactccagtcccttcccgcaagcctggcggac

SEQ ID NO: 195 - DNA, 54 bp, synthetic construct
  source 1..54: mol_type = other DNA
ggagtctgcgtagcatgtgctaccaccgctataaataaagcagacccactccag

SEQ ID NO: 196 - DNA, 51 bp, synthetic construct
  source 1..51: mol_type = other DNA
ggagtctgcgtagcatgtgctaccaccgctataaatgcatgagacccactc

SEQ ID NO: 197 - DNA, 54 bp, synthetic construct
  source 1..54: mol_type = other DNA
cacggagtctgcgcagtatgtgctaccaccgctataaatgcatgagacccactc

SEQ ID NO: 198 - DNA, 51 bp, synthetic construct
  source 1..51: mol_type = other DNA
ggtggtagcacatgctacgcagactccgtgaagggctgcttcaccatctcc

SEQ ID NO: 199 - DNA, 54 bp, synthetic construct
  source 1..54: mol_type = other DNA
ggtggtagcacatgctacgcagactccgtgaagggccgatgcaccatctccaga

SEQ ID NO: 200 - DNA, 48 bp, synthetic construct
  source 1..48: mol_type = other DNA
ggtagcacatactgcgcagactccgtgaagggctgcttcaccatctcc

SEQ ID NO: 201 - DNA, 51 bp, synthetic construct
  source 1..51: mol_type = other DNA
ggtagcacatactgcgcagactccgtgaagggccgatgcaccatctccaga

SEQ ID NO: 202 - DNA, 54 bp, synthetic construct
  source 1..54: mol_type = other DNA
ggtagcacatactgcgcagactccgtgaagggccgattctgcatctccagagac

SEQ ID NO: 203 - DNA, 47 bp, synthetic construct
  source 1..47: mol_type = other DNA
gtagcacatactactgcgactccgtgaagggctgcttcaccatctcc

SEQ ID NO: 204 - DNA, 50 bp, synthetic construct
  source 1..50: mol_type = other DNA
gtagcacatactactgcgactccgtgaagggccgatgcaccatctccaga

SEQ ID NO: 205 - DNA, 53 bp, synthetic construct
  source 1..53: mol_type = other DNA
gtagcacatactactgcgactccgtgaagggccgattctgcatctccagagac

SEQ ID NO: 206 - DNA, 56 bp, synthetic construct
  source 1..56: mol_type = other DNA
gtagcacatactactgcgactccgtgaagggccgattcacctgctccagagacaat

SEQ ID NO: 207 - DNA, 43 bp, synthetic construct
  source 1..43: mol_type = other DNA
cacatactacgcatgctccgtgaagggctgcttcaccatctcc

SEQ ID NO: 208 - DNA, 46 bp, synthetic construct
  source 1..46: mol_type = other DNA
cacatactacgcatgctccgtgaagggccgatgcaccatctccaga

SEQ ID NO: 209 - DNA, 49 bp, synthetic construct
  source 1..49: mol_type = other DNA
cacatactacgcatgctccgtgaagggccgattctgcatctccagagac

SEQ ID NO: 210 - DNA, 52 bp, synthetic construct
  source 1..52: mol_type = other DNA
cacatactacgcatgctccgtgaagggccgattcacctgctccagagacaat

SEQ ID NO: 211 - DNA, 55 bp, synthetic construct
  source 1..55: mol_type = other DNA
cacatactacgcatgctccgtgaagggccgattcaccatctgcagagacaattcc

SEQ ID NO: 212 - DNA, 41 bp, synthetic construct
  source 1..41: mol_type = other DNA
catactacgcagactgcgtgaagggctgcttcaccatctcc

SEQ ID NO: 213 - DNA, 44 bp, synthetic construct
  source 1..44: mol_type = other DNA
catactacgcagactgcgtgaagggccgatgcaccatctccaga

SEQ ID NO: 214 - DNA, 47 bp, synthetic construct
  source 1..47: mol_type = other DNA
catactacgcagactgcgtgaagggccgattctgcatctccagagac

SEQ ID NO: 215 - DNA, 50 bp, synthetic construct
  source 1..50: mol_type = other DNA
catactacgcagactgcgtgaagggccgattcacctgctccagagacaat

SEQ ID NO: 216 - DNA, 53 bp, synthetic construct
  source 1..53: mol_type = other DNA
catactacgcagactgcgtgaagggccgattcaccatctgcagagacaattcc

SEQ ID NO: 217 - DNA, 56 bp, synthetic construct
  source 1..56: mol_type = other DNA
catactacgcagactgcgtgaagggccgattcaccatctcctgcgacaattccaag

SEQ ID NO: 218 - DNA, 40 bp, synthetic construct
  source 1..40: mol_type = other DNA
ctacgcagactcctgcaagggctgcttcaccatctccaga

SEQ ID NO: 219 - DNA, 40 bp, synthetic construct
  source 1..40: mol_type = other DNA
ctacgcagactcctgcaagggccgatgcaccatctccaga

SEQ ID NO: 220 - DNA, 43 bp, synthetic construct
  source 1..43: mol_type = other DNA
ctacgcagactcctgcaagggccgattctgcatctccagagac

SEQ ID NO: 221 - DNA, 46 bp, synthetic construct
  source 1..46: mol_type = other DNA
ctacgcagactcctgcaagggccgattcacctgctccagagacaat

SEQ ID NO: 222 - DNA, 49 bp, synthetic construct
  source 1..49: mol_type = other DNA
ctacgcagactcctgcaagggccgattcaccatctgcagagacaattcc

SEQ ID NO: 223 - DNA, 52 bp, synthetic construct
  source 1..52: mol_type = other DNA
ctacgcagactcctgcaagggccgattcaccatctcctgcgacaattccaag

SEQ ID NO: 224 - DNA, 55 bp, synthetic construct
  source 1..55: mol_type = other DNA
ctacgcagactcctgcaagggccgattcaccatctccagatgcaattccaagaac

SEQ ID NO: 225 - DNA, 40 bp, synthetic construct
  source 1..40: mol_type = other DNA
ctacgcagactccgtgtgcggctgcttcaccatctccaga

SEQ ID NO: 226 - DNA, 43 bp, synthetic construct
  source 1..43: mol_type = other DNA
ctacgcagactccgtgtgcggccgatgcaccatctccagagac

SEQ ID NO: 227 - DNA, 39 bp, synthetic construct
  source 1..39: mol_type = other DNA
gcagactccgtgtgcggccgattctgcatctccagagac

SEQ ID NO: 228 - DNA, 42 bp, synthetic construct
  source 1..42: mol_type = other DNA
gcagactccgtgtgcggccgattcacctgctccagagacaat

SEQ ID NO: 229 - DNA, 45 bp, synthetic construct
  source 1..45: mol_type = other DNA
gcagactccgtgtgcggccgattcaccatctgcagagacaattcc

SEQ ID NO: 230 - DNA, 48 bp, synthetic construct
  source 1..48: mol_type = other DNA
gcagactccgtgtgcggccgattcaccatctcctgcgacaattccaag

SEQ ID NO: 231 - DNA, 51 bp, synthetic construct
  source 1..51: mol_type = other DNA
gcagactccgtgtgcggccgattcaccatctccagatgcaattccaagaac

SEQ ID NO: 232 - DNA, 54 bp, synthetic construct
  source 1..54: mol_type = other DNA
gcagactccgtgtgcggccgattcaccatctccagagactgctccaagaacacg

SEQ ID NO: 233 - DNA, 37 bp, synthetic construct
  source 1..37: mol_type = other DNA
cgcagactccgtgaagtgctgcttcaccatctccaga

SEQ ID NO: 234 - DNA, 40 bp, synthetic construct
  source 1..40: mol_type = other DNA
cgcagactccgtgaagtgccgatgcaccatctccagagac

SEQ ID NO: 235 - DNA, 43 bp, synthetic construct
  source 1..43: mol_type = other DNA
cgcagactccgtgaagtgccgattctgcatctccagagacaat

SEQ ID NO: 236 - DNA, 39 bp, synthetic construct
  source 1..39: mol_type = other DNA
gactccgtgaagtgccgattcacctgctccagagacaat

SEQ ID NO: 237 - DNA, 42 bp, synthetic construct
  source 1..42: mol_type = other DNA
gactccgtgaagtgccgattcaccatctgcagagacaattcc

SEQ ID NO: 238 - DNA, 45 bp, synthetic construct
  source 1..45: mol_type = other DNA
gactccgtgaagtgccgattcaccatctcctgcgacaattccaag

SEQ ID NO: 239 - DNA, 48 bp, synthetic construct
  source 1..48: mol_type = other DNA
gactccgtgaagtgccgattcaccatctccagatgcaattccaagaac

SEQ ID NO: 240 - DNA, 51 bp, synthetic construct
  source 1..51: mol_type = other DNA
gactccgtgaagtgccgattcaccatctccagagactgctccaagaacacg

SEQ ID NO: 241 - DNA, 54 bp, synthetic construct
  source 1..54: mol_type = other DNA
gactccgtgaagtgccgattcaccatctccagagacaattgcaagaacacgctg

SEQ ID NO: 242 - DNA, 54 bp, synthetic construct
  source 1..54: mol_type = other DNA
aagatacagcgtgcacttggaattgtctctggagatggtgcatcggcccttcac

SEQ ID NO: 243 - DNA, 52 bp, synthetic construct
  source 1..52: mol_type = other DNA
aagatacagcgtgcacttggaattgtctctggagatgcagaatcggcccttc

SEQ ID NO: 244 - DNA, 55 bp, synthetic construct
  source 1..55: mol_type = other DNA
ttgaagatacaggcagttcttggaattgtctctggagatgcagaatcggcccttc

SEQ ID NO: 245 - DNA, 48 bp, synthetic construct
  source 1..48: mol_type = other DNA
aagatacagcgtgcacttggaattgtctctggagcaggtgaatcggcc

SEQ ID NO: 246 - DNA, 51 bp, synthetic construct
  source 1..51: mol_type = other DNA
ttgaagatacaggcagttcttggaattgtctctggagcaggtgaatcggcc

SEQ ID NO: 247 - DNA, 54 bp, synthetic construct
  source 1..54: mol_type = other DNA
catttgaagatagcacgtgttcttggaattgtctctggagcaggtgaatcggcc

SEQ ID NO: 248 - DNA, 45 bp, synthetic construct
  source 1..45: mol_type = other DNA
aagatacagcgtgcacttggaattgtctctgcagatggtgaatcg

SEQ ID NO: 249 - DNA, 48 bp, synthetic construct
  source 1..48: mol_type = other DNA
ttgaagatacaggcagttcttggaattgtctctgcagatggtgaatcg

SEQ ID NO: 250 - DNA, 51 bp, synthetic construct
  source 1..51: mol_type = other DNA
catttgaagatagcacgtgttcttggaattgtctctgcagatggtgaatcg

SEQ ID NO: 251 - DNA, 54 bp, synthetic construct
  source 1..54: mol_type = other DNA
gttcatttgaaggcacagcgtgttcttggaattgtctctgcagatggtgaatcg

SEQ ID NO: 252 - DNA, 44 bp, synthetic construct
  source 1..44: mol_type = other DNA
aagatacagcgtgcacttggaattgtcgcaggagatggtgaatc

SEQ ID NO: 253 - DNA, 47 bp, synthetic construct
  source 1..47: mol_type = other DNA
ttgaagatacaggcagttcttggaattgtcgcaggagatggtgaatc

SEQ ID NO: 254 - DNA, 50 bp, synthetic construct
  source 1..50: mol_type = other DNA
catttgaagatagcacgtgttcttggaattgtcgcaggagatggtgaatc

SEQ ID NO: 255 - DNA, 53 bp, synthetic construct
  source 1..53: mol_type = other DNA
gttcatttgaaggcacagcgtgttcttggaattgtcgcaggagatggtgaatc

SEQ ID NO: 256 - DNA, 56 bp, synthetic construct
  source 1..56: mol_type = other DNA
gctgttcatttggcaatacagcgtgttcttggaattgtcgcaggagatggtgaatc

SEQ ID NO: 257 - DNA, 40 bp, synthetic construct
  source 1..40: mol_type = other DNA
aagatacagcgtgcacttggaattgcatctggagatggtg

SEQ ID NO: 258 - DNA, 43 bp, synthetic construct
  source 1..43: mol_type = other DNA
ttgaagatacaggcagttcttggaattgcatctggagatggtg

SEQ ID NO: 259 - DNA, 46 bp, synthetic construct
  source 1..46: mol_type = other DNA
catttgaagatagcacgtgttcttggaattgcatctggagatggtg

SEQ ID NO: 260 - DNA, 49 bp, synthetic construct
  source 1..49: mol_type = other DNA
gttcatttgaaggcacagcgtgttcttggaattgcatctggagatggtg

SEQ ID NO: 261 - DNA, 52 bp, synthetic construct
  source 1..52: mol_type = other DNA
gctgttcatttggcaatacagcgtgttcttggaattgcatctggagatggtg

SEQ ID NO: 262 - DNA, 55 bp, synthetic construct
  source 1..55: mol_type = other DNA
caggctgttcatgcaaagatacagcgtgttcttggaattgcatctggagatggtg

SEQ ID NO: 263 - DNA, 37 bp, synthetic construct
  source 1..37: mol_type = other DNA
aagatacagcgtgcacttggagcagtctctggagatg

SEQ ID NO: 264 - DNA, 40 bp, synthetic construct
  source 1..40: mol_type = other DNA
ttgaagatacaggcagttcttggagcagtctctggagatg

SEQ ID NO: 265 - DNA, 43 bp, synthetic construct
  source 1..43: mol_type = other DNA
catttgaagatagcacgtgttcttggagcagtctctggagatg

SEQ ID NO: 266 - DNA, 46 bp, synthetic construct
  source 1..46: mol_type = other DNA
gttcatttgaaggcacagcgtgttcttggagcagtctctggagatg

SEQ ID NO: 267 - DNA, 49 bp, synthetic construct
  source 1..49: mol_type = other DNA
gctgttcatttggcaatacagcgtgttcttggagcagtctctggagatg

SEQ ID NO: 268 - DNA, 52 bp, synthetic construct
  source 1..52: mol_type = other DNA
caggctgttcatgcaaagatacagcgtgttcttggagcagtctctggagatg

SEQ ID NO: 269 - DNA, 55 bp, synthetic construct
  source 1..55: mol_type = other DNA
tctcaggctgttgcattgaagatacagcgtgttcttggagcagtctctggagatg

SEQ ID NO: 270 - DNA, 34 bp, synthetic construct
  source 1..34: mol_type = other DNA
aagatacagcgtgcacttgcaattgtctctggag

SEQ ID NO: 271 - DNA, 37 bp, synthetic construct
  source 1..37: mol_type = other DNA
ttgaagatacaggcagttcttgcaattgtctctggag

SEQ ID NO: 272 - DNA, 40 bp, synthetic construct
  source 1..40: mol_type = other DNA
catttgaagatagcacgtgttcttgcaattgtctctggag

SEQ ID NO: 273 - DNA, 43 bp, synthetic construct
  source 1..43: mol_type = other DNA
gttcatttgaaggcacagcgtgttcttgcaattgtctctggag

SEQ ID NO: 274 - DNA, 46 bp, synthetic construct
  source 1..46: mol_type = other DNA
gctgttcatttggcaatacagcgtgttcttgcaattgtctctggag

SEQ ID NO: 275 - DNA, 49 bp, synthetic construct
  source 1..49: mol_type = other DNA
caggctgttcatgcaaagatacagcgtgttcttgcaattgtctctggag

SEQ ID NO: 276 - DNA, 52 bp, synthetic construct
  source 1..52: mol_type = other DNA
tctcaggctgttgcattgaagatacagcgtgttcttgcaattgtctctggag

SEQ ID NO: 277 - DNA, 55 bp, synthetic construct
  source 1..55: mol_type = other DNA
agctctcaggctgcacatttgaagatacagcgtgttcttgcaattgtctctggag

SEQ ID NO: 278 - DNA, 31 bp, synthetic construct
  source 1..31: mol_type = other DNA
aagatacagcgtgcagcaggaattgtctctg

SEQ ID NO: 279 - DNA, 34 bp, synthetic construct
  source 1..34: mol_type = other DNA
ttgaagatacaggcagttgcaggaattgtctctg

SEQ ID NO: 280 - DNA, 37 bp, synthetic construct
  source 1..37: mol_type = other DNA
catttgaagatagcacgtgttgcaggaattgtctctg

SEQ ID NO: 281 - DNA, 40 bp, synthetic construct
  source 1..40: mol_type = other DNA
gttcatttgaaggcacagcgtgttgcaggaattgtctctg

SEQ ID NO: 282 - DNA, 43 bp, synthetic construct
  source 1..43: mol_type = other DNA
gctgttcatttggcaatacagcgtgttgcaggaattgtctctg

SEQ ID NO: 283 - DNA, 46 bp, synthetic construct
  source 1..46: mol_type = other DNA
caggctgttcatgcaaagatacagcgtgttgcaggaattgtctctg

SEQ ID NO: 284 - DNA, 49 bp, synthetic construct
  source 1..49: mol_type = other DNA
tctcaggctgttgcattgaagatacagcgtgttgcaggaattgtctctg

SEQ ID NO: 285 - DNA, 52 bp, synthetic construct
  source 1..52: mol_type = other DNA
agctctcaggctgcacatttgaagatacagcgtgttgcaggaattgtctctg

SEQ ID NO: 286 - DNA, 55 bp, synthetic construct
  source 1..55: mol_type = other DNA
ctcagctctcaggcagttcatttgaagatacagcgtgttgcaggaattgtctctg

SEQ ID NO: 287 - DNA, 55 bp, synthetic construct
  source 1..55: mol_type = other DNA
cacagccgtgtcgcaagctctcaggctgttcatttgaaggcacagcgtgttcttg

SEQ ID NO: 288 - DNA, 52 bp, synthetic construct
  source 1..52: mol_type = other DNA
cacagccgtgtcgcaagctctcaggctgttcatttggcaatacagcgtgttc

SEQ ID NO: 289 - DNA, 55 bp, synthetic construct
  source 1..55: mol_type = other DNA
atacacagccgtgcactcagctctcaggctgttcatttggcaatacagcgtgttc

SEQ ID NO: 290 - DNA, 49 bp, synthetic construct
  source 1..49: mol_type = other DNA
cacagccgtgtcgcaagctctcaggctgttcatgcaaagatacagcgtg

SEQ ID NO: 291 - DNA, 52 bp, synthetic construct
  source 1..52: mol_type = other DNA
atacacagccgtgcactcagctctcaggctgttcatgcaaagatacagcgtg

SEQ ID NO: 292 - DNA, 55 bp, synthetic construct
  source 1..55: mol_type = other DNA
gtaatacacagcgcagtcctcagctctcaggctgttcatgcaaagatacagcgtg

SEQ ID NO: 293 - DNA, 45 bp, synthetic construct
  source 1..45: mol_type = other DNA
cacagccgtgtcgcaagctctcaggctgttgcattgaagatacag

SEQ ID NO: 294 - DNA, 48 bp, synthetic construct
  source 1..48: mol_type = other DNA
atacacagccgtgcactcagctctcaggctgttgcattgaagatacag

SEQ ID NO: 295 - DNA, 51 bp, synthetic construct
  source 1..51: mol_type = other DNA
gtaatacacagcgcagtcctcagctctcaggctgttgcattgaagatacag

SEQ ID NO: 296 - DNA, 54 bp, synthetic construct
  source 1..54: mol_type = other DNA
tgcgtaatacacgcacgtgtcctcagctctcaggctgttgcattgaagatacag

SEQ ID NO: 297 - DNA, 43 bp, synthetic construct
  source 1..43: mol_type = other DNA
cacagccgtgtcgcaagctctcaggctgcacatttgaagatac

SEQ ID NO: 298 - DNA, 46 bp, synthetic construct
  source 1..46: mol_type = other DNA
atacacagccgtgcactcagctctcaggctgcacatttgaagatac

SEQ ID NO: 299 - DNA, 49 bp, synthetic construct
  source 1..49: mol_type = other DNA
gtaatacacagcgcagtcctcagctctcaggctgcacatttgaagatac

SEQ ID NO: 300 - DNA, 52 bp, synthetic construct
  source 1..52: mol_type = other DNA
tgcgtaatacacgcacgtgtcctcagctctcaggctgcacatttgaagatac

SEQ ID NO: 301 - DNA, 55 bp, synthetic construct
  source 1..55: mol_type = other DNA
cgctgcgtaatagcaagccgtgtcctcagctctcaggctgcacatttgaagatac

SEQ ID NO: 302 - DNA, 39 bp, synthetic construct
  source 1..39: mol_type = other DNA
cacagccgtgtcgcaagctctcaggcagttcatttgaag

SEQ ID NO: 303 - DNA, 42 bp, synthetic construct
  source 1..42: mol_type = other DNA
atacacagccgtgcactcagctctcaggcagttcatttgaag

SEQ ID NO: 304 - DNA, 45 bp, synthetic construct
  source 1..45: mol_type = other DNA
gtaatacacagcgcagtcctcagctctcaggcagttcatttgaag

SEQ ID NO: 305 - DNA, 48 bp, synthetic construct
  source 1..48: mol_type = other DNA
tgcgtaatacacgcacgtgtcctcagctctcaggcagttcatttgaag

SEQ ID NO: 306 - DNA, 51 bp, synthetic construct
  source 1..51: mol_type = other DNA
cgctgcgtaatagcaagccgtgtcctcagctctcaggcagttcatttgaag

SEQ ID NO: 307 - DNA, 54 bp, synthetic construct
  source 1..54: mol_type = other DNA
tctcgctgcgtagcacacagccgtgtcctcagctctcaggcagttcatttgaag

SEQ ID NO: 308 - DNA, 42 bp, synthetic construct
  source 1..42: mol_type = other DNA
atacacagccgtgtcgcaagctctgcagctgttcatttgaag

SEQ ID NO: 309 - DNA, 39 bp, synthetic construct
  source 1..39: mol_type = other DNA
atacacagccgtgcactcagctctgcagctgttcatttg

SEQ ID NO: 310 - DNA, 42 bp, synthetic construct
  source 1..42: mol_type = other DNA
gtaatacacagcgcagtcctcagctctgcagctgttcatttg

SEQ ID NO: 311 - DNA, 45 bp, synthetic construct
  source 1..45: mol_type = other DNA
tgcgtaatacacgcacgtgtcctcagctctgcagctgttcatttg

SEQ ID NO: 312 - DNA, 48 bp, synthetic construct
  source 1..48: mol_type = other DNA
cgctgcgtaatagcaagccgtgtcctcagctctgcagctgttcatttg

SEQ ID NO: 313 - DNA, 51 bp, synthetic construct
  source 1..51: mol_type = other DNA
tctcgctgcgtagcacacagccgtgtcctcagctctgcagctgttcatttg

SEQ ID NO: 314 - DNA, 54 bp, synthetic construct
  source 1..54: mol_type = other DNA
ctctctcgctgcgcaatacacagccgtgtcctcagctctgcagctgttcatttg

SEQ ID NO: 315 - DNA, 39 bp, synthetic construct
  source 1..39: mol_type = other DNA
atacacagccgtgtcgcaagcgcacaggctgttcatttg

SEQ ID NO: 316 - DNA, 42 bp, synthetic construct
  source 1..42: mol_type = other DNA
gtaatacacagccgtgcactcagcgcacaggctgttcatttg

SEQ ID NO: 317 - DNA, 42 bp, synthetic construct
  source 1..42: mol_type = other DNA
gtaatacacagcgcagtcctcagcgcacaggctgttcatttg

SEQ ID NO: 318 - DNA, 45 bp, synthetic construct
  source 1..45: mol_type = other DNA
tgcgtaatacacgcacgtgtcctcagcgcacaggctgttcatttg

SEQ ID NO: 319 - DNA, 48 bp, synthetic construct
  source 1..48: mol_type = other DNA
cgctgcgtaatagcaagccgtgtcctcagcgcacaggctgttcatttg

SEQ ID NO: 320 - DNA, 51 bp, synthetic construct
  source 1..51: mol_type = other DNA
tctcgctgcgtagcacacagccgtgtcctcagcgcacaggctgttcatttg

SEQ ID NO: 321 - DNA, 54 bp, synthetic construct
  source 1..54: mol_type = other DNA
ctctctcgctgcgcaatacacagccgtgtcctcagcgcacaggctgttcatttg

SEQ ID NO: 322 - DNA, 57 bp, synthetic construct
  source 1..57: mol_type = other DNA
gctctctctcgcgcagtaatacacagccgtgtcctcagcgcacaggctgttcatttg

SEQ ID NO: 323 - DNA, 39 bp, synthetic construct
  source 1..39: mol_type = other DNA
atacacagccgtgtcgcagcatctcaggctgttcatttg

SEQ ID NO: 324 - DNA, 42 bp, synthetic construct
  source 1..42: mol_type = other DNA
gtaatacacagccgtgcactcgcatctcaggctgttcatttg

SEQ ID NO: 325 - DNA, 45 bp, synthetic construct
  source 1..45: mol_type = other DNA
tgcgtaatacacagcgcagtcctcgcatctcaggctgttcatttg

SEQ ID NO: 326 - DNA, 40 bp, synthetic construct
  source 1..40: mol_type = other DNA
tgcgtaatacacgcacgtgtcctcgcatctcaggctgttc

SEQ ID NO: 327 - DNA, 43 bp, synthetic construct
  source 1..43: mol_type = other DNA
cgctgcgtaatagcaagccgtgtcctcgcatctcaggctgttc

SEQ ID NO: 328 - DNA, 46 bp, synthetic construct
  source 1..46: mol_type = other DNA
tctcgctgcgtagcacacagccgtgtcctcgcatctcaggctgttc

SEQ ID NO: 329 - DNA, 49 bp, synthetic construct
  source 1..49: mol_type = other DNA
ctctctcgctgcgcaatacacagccgtgtcctcgcatctcaggctgttc

SEQ ID NO: 330 - DNA, 52 bp, synthetic construct
  source 1..52: mol_type = other DNA
gctctctctcgcgcagtaatacacagccgtgtcctcgcatctcaggctgttc

SEQ ID NO: 331 - protein, 125 aa, synthetic construct
QVKLEESGGGLVQAGGSLRLSCAASGRTFNTLSMGWFRQAPGKEREFVAAVSRSGGSTYYADSVKGRFTISRDNAKNTVYLQMNSLKPEDTAVYYCAAAATKSNTTAYRLSFDYWGQGTQVTVSS

SEQ ID NO: 332 - protein, 125 aa, synthetic construct
QVKLEESGGGLVQAGGSLRLCCAASGRTFNTLSMGWFRQAPGKEREFVAAVSRSGGSTYYADSVKGRFTISRDNAKNTVCLQMNSLKPEDTAVYYCAAAATKSNTTAYRLSFDYWGQGTQVTVSS

SEQ ID NO: 333 - protein, 125 aa, synthetic construct
QVKLEESGGGLVQAGGSLRLSCCASGRTFNTLSMGWFRQAPGKEREFVAAVSRSGGSTYYADSVKGRFTISRDNAKNCVYLQMNSLKPEDTAVYYCAAAATKSNTTAYRLSFDYWGQGTQVTVSS

SEQ ID NO: 334 - protein, 125 aa, synthetic construct
QVKLEESGGGLVQAGGSLRLSCAASGRTFNTLSMGWFRQCPGKEREFVAAVSRSGGSTYYADSVKGRFTISRDNAKNTVYLQMNSLKPEDTCVYYCAAAATKSNTTAYRLSFDYWGQGTQVTVSS

SEQ ID NO: 335 - protein, 125 aa, synthetic construct
QVKCEESGGGLVQAGGSLRLSCACSGRTFNTLSMGWFRQAPGKEREFVAAVSRSGGSTYYADSVKGRFTISRDNAKNTVYLQMNSLKPEDTAVYYCAAAATKSNTTAYRLSFDYWGQGTQVTVSS

SEQ ID NO: 336 - protein, 125 aa, synthetic construct
QVKCEESGGGLVQAGGSLRLSCAASGRTFNTLSMGWFRQAPGKEREFVAAVSRSGGSTYYADSVKGRFTISRDNAKNTVYLQMNSLKPEDTAVYYCAAAATKSNTTAYRLSFDYCGQGTQVTVSS

SEQ ID NO: 337 - protein, 125 aa, synthetic construct
QVKLCESGGGLVQAGGSLRLSCAASGRTFNTLSMGWFRQAPGKEREFVAAVSRSGGSTYYADSVKGRFTISRDNAKNTVYLQMNSLKPEDTAVYYCAAAATKSNTTAYRLSFDYWGCGTQVTVSS

SEQ ID NO: 338 - protein, 125 aa, synthetic construct
QVKLECSGGGLVQAGGSLRLSCAASGRTFNTLSMGWFRQAPGKEREFVAAVSRSGGSTYYADSVKGRFTISRDNAKNTVYLQMNSLKPEDTAVYYCAAAATKSNTTAYRLSFDYWCQGTQVTVSS

SEQ ID NO: 339 - protein, 124 aa, synthetic construct
QVKLGESGGGLVQAGGSLRLSCAASGRTFSMYRMGWFRQAPGKEREFVGVITRNGSSTYYADSVKGRFTISRDNAKNTVYLQMNSLKPEDTALYYCAATSGSSYLDAAHVYDYWGQGTQVTVSS

SEQ ID NO: 340 - protein, 124 aa, synthetic construct
QVKLGESGGGLVQAGGSLRLCCAASGRTFSMYRMGWFRQAPGKEREFVGVITRNGSSTYYADSVKGRFTISRDNAKNTVCLQMNSLKPEDTALYYCAATSGSSYLDAAHVYDYWGQGTQVTVSS

SEQ ID NO: 341 - protein, 124 aa, synthetic construct
QVKLGESGGGLVQAGGSLRLSCCASGRTFSMYRMGWFRQAPGKEREFVGVITRNGSSTYYADSVKGRFTISRDNAKNCVYLQMNSLKPEDTALYYCAATSGSSYLDAAHVYDYWGQGTQVTVSS

SEQ ID NO: 342 - protein, 124 aa, synthetic construct
QVKLGESGGGLVQAGGSLRLSCAASGRTFSMYRMGWFRQCPGKEREFVGVITRNGSSTYYADSVKGRFTISRDNAKNTVYLQMNSLKPEDTCLYYCAATSGSSYLDAAHVYDYWGQGTQVTVSS

SEQ ID NO: 343 - protein, 124 aa, synthetic construct
QVKCGESGGGLVQAGGSLRLSCACSGRTFSMYRMGWFRQAPGKEREFVGVITRNGSSTYYADSVKGRFTISRDNAKNTVYLQMNSLKPEDTALYYCAATSGSSYLDAAHVYDYWGQGTQVTVSS

SEQ ID NO: 344 - protein, 124 aa, synthetic construct
QVKCGESGGGLVQAGGSLRLSCAASGRTFSMYRMGWFRQAPGKEREFVGVITRNGSSTYYADSVKGRFTISRDNAKNTVYLQMNSLKPEDTALYYCAATSGSSYLDAAHVYDYCGQGTQVTVSS

SEQ ID NO: 345 - protein, 124 aa, synthetic construct
QVKLCESGGGLVQAGGSLRLSCAASGRTFSMYRMGWFRQAPGKEREFVGVITRNGSSTYYADSVKGRFTISRDNAKNTVYLQMNSLKPEDTALYYCAATSGSSYLDAAHVYDYWGCGTQVTVSS

SEQ ID NO: 346 - protein, 124 aa, synthetic construct
QVKLGCSGGGLVQAGGSLRLSCAASGRTFSMYRMGWFRQAPGKEREFVGVITRNGSSTYYADSVKGRFTISRDNAKNTVYLQMNSLKPEDTALYYCAATSGSSYLDAAHVYDYWCQGTQVTVSS

SEQ ID NO: 347 - protein, 123 aa, synthetic construct
QVQLVESGGGLAQAGGSLRLSCAASGRTFSMDPMAWFRQPPGKEREFVAAGSSTGRTTYYADSVKGRFTISRDNAKNTVYLQMNSLKPEDTAVYYCAAAPYGANWYRDEYAYWGQGTQVTVSS

SEQ ID NO: 348 - protein, 123 aa, synthetic construct
QVQLVESGGGLAQAGGSLRLCCAASGRTFSMDPMAWFRQPPGKEREFVAAGSSTGRTTYYADSVKGRFTISRDNAKNTVCLQMNSLKPEDTAVYYCAAAPYGANWYRDEYAYWGQGTQVTVSS

SEQ ID NO: 349 - protein, 123 aa, synthetic construct
QVQLVESGGGLAQAGGSLRLSCCASGRTFSMDPMAWFRQPPGKEREFVAAGSSTGRTTYYADSVKGRFTISRDNAKNCVYLQMNSLKPEDTAVYYCAAAPYGANWYRDEYAYWGQGTQVTVSS

SEQ ID NO: 350 - protein, 123 aa, synthetic construct
QVQLVESGGGLAQAGGSLRLSCAASGRTFSMDPMAWFRQCPGKEREFVAAGSSTGRTTYYADSVKGRFTISRDNAKNTVYLQMNSLKPEDTCVYYCAAAPYGANWYRDEYAYWGQGTQVTVSS

SEQ ID NO: 351 - protein, 123 aa, synthetic construct
QVQCVESGGGLAQAGGSLRLSCACSGRTFSMDPMAWFRQPPGKEREFVAAGSSTGRTTYYADSVKGRFTISRDNAKNTVYLQMNSLKPEDTAVYYCAAAPYGANWYRDEYAYWGQGTQVTVSS

SEQ ID NO: 352 - protein, 123 aa, synthetic construct
QVQCVESGGGLAQAGGSLRLSCAASGRTFSMDPMAWFRQPPGKEREFVAAGSSTGRTTYYADSVKGRFTISRDNAKNTVYLQMNSLKPEDTAVYYCAAAPYGANWYRDEYAYCGQGTQVTVSS

SEQ ID NO: 353 - protein, 123 aa, synthetic construct
QVQLCESGGGLAQAGGSLRLSCAASGRTFSMDPMAWFRQPPGKEREFVAAGSSTGRTTYYADSVKGRFTISRDNAKNTVYLQMNSLKPEDTAVYYCAAAPYGANWYRDEYAYWGCGTQVTVSS

SEQ ID NO: 354 - protein, 123 aa, synthetic construct
QVQLVCSGGGLAQAGGSLRLSCAASGRTFSMDPMAWFRQPPGKEREFVAAGSSTGRTTYYADSVKGRFTISRDNAKNTVYLQMNSLKPEDTAVYYCAAAPYGANWCRDEYAYWGQGTQVTVSS

SEQ ID NO: 355 - protein, 124 aa, synthetic construct [sequence omitted]

SEQ ID NO: 356 - protein, 124 aa, synthetic construct
QVQLVESGGGLVQAGGSLRLCCAASIRSFSNRNMGWFRQPPGKEREFVAGISWGGGSTRYADSVKGRFTISRDNAKKTVCLQMNSLKPEDTAVYYCAAEFGHNIATSSDEYDYWGQGTQVTVSS

SEQ ID NO: 357 - protein, 124 aa, synthetic construct
QVQLVESGGGLVQAGGSLRLSCCASIRSFSNRNMGWFRQPPGKEREFVAGISWGGGSTRYADSVKGRFTISRDNAKKCVYLQMNSLKPEDTAVYYCAAEFGHNIATSSDEYDYWGQGTQVTVSS

SEQ ID NO: 358 - protein, 124 aa, synthetic construct
QVQLVESGGGLVQAGGSLRLSCAASIRSFSNRNMGWFRQCPGKEREFVAGISWGGGSTRYADSVKGRFTISRDNAKKTVYLQMNSLKPEDTCVYYCAAEFGHNIATSSDEYDYWGQGTQVTVSS

SEQ ID NO: 359 - protein, 124 aa, synthetic construct
QVQCVESGGGLVQAGGSLRLSCACSIRSFSNRNMGWFRQPPGKEREFVAGISWGGGSTRYADSVKGRFTISRDNAKKTVYLQMNSLKPEDTAVYYCAAEFGHNIATSSDEYDYWGQGTQVTVSS

SEQ ID NO: 360 - protein, 124 aa, synthetic construct
QVQCVESGGGLVQAGGSLRLSCAASIRSFSNRNMGWFRQPPGKEREFVAGISWGGGSTRYADSVKGRFTISRDNAKKTVYLQMNSLKPEDTAVYYCAAEFGHNIATSSDEYDYCGQGTQVTVSS

SEQ ID NO: 361 - protein, 124 aa, synthetic construct
QVQLCESGGGLVQAGGSLRLSCAASIRSFSNRNMGWFRQPPGKEREFVAGISWGGGSTRYADSVKGRFTISRDNAKKTVYLQMNSLKPEDTAVYYCAAEFGHNIATSSDEYDYWGCGTQVTVSS

SEQ ID NO: 362 - protein, 124 aa, synthetic construct
QVQLVCSGGGLVQAGGSLRLSCAASIRSFSNRNMGWFRQPPGKEREFVAGISWGGGSTRYADSVKGRFTISRDNAKKTVYLQMNSLKPEDTAVYYCAAEFGHNIATSSDEYDYWCQGTQVTVSS

SEQ ID NO: 363 - protein, 124 aa, synthetic construct
QVKLEESGGGLVQAGGSLRLSCAASERTFSRYPVAWFRQAPGAEREFVAVISSTGTSTYYADSVKGRFTISRDNAKVTVYLQMNNLKREDTAVYFCAVNSQRTRLQDPNEYDYWGQGTQVTVSS

SEQ ID NO: 364 - protein, 124 aa, synthetic construct
QVKLEESGGGLVQAGGSLRLCCAASERTFSRYPVAWFRQAPGAEREFVAVISSTGTSTYYADSVKGRFTISRDNAKVTVCLQMNNLKREDTAVYFCAVNSQRTRLQDPNEYDYWGQGTQVTVSS

SEQ ID NO: 365 - protein, 124 aa, synthetic construct
QVKLEESGGGLVQAGGSLRLSCCASERTFSRYPVAWFRQAPGAEREFVAVISSTGTSTYYADSVKGRFTISRDNAKVCVYLQMNNLKREDTAVYFCAVNSQRTRLQDPNEYDYWGQGTQVTVSS

SEQ ID NO: 366 - protein, 124 aa, synthetic construct
QVKLEESGGGLVQAGGSLRLSCAASERTFSRYPVAWFRQCPGAEREFVAVISSTGTSTYYADSVKGRFTISRDNAKVTVYLQMNNLKREDTCVYFCAVNSQRTRLQDPNEYDYWGQGTQVTVSS

SEQ ID NO: 367 - protein, 124 aa, synthetic construct
QVKCEESGGGLVQAGGSLRLSCACSERTFSRYPVAWFRQAPGAEREFVAVISSTGTSTYYADSVKGRFTISRDNAKVTVYLQMNNLKREDTAVYFCAVNSQRTRLQDPNEYDYWGQGTQVTVSS

SEQ ID NO: 368 - protein, 124 aa, synthetic construct
QVKCEESGGGLVQAGGSLRLSCAASERTFSRYPVAWFRQAPGAEREFVAVISSTGTSTYYADSVKGRFTISRDNAKVTVYLQMNNLKREDTAVYFCAVNSQRTRLQDPNEYDYCGQGTQVTVSS

SEQ ID NO: 369 - protein, 124 aa, synthetic construct
QVKLCESGGGLVQAGGSLRLSCAASERTFSRYPVAWFRQAPGAEREFVAVISSTGTSTYYADSVKGRFTISRDNAKVTVYLQMNNLKREDTAVYFCAVNSQRTRLQDPNEYDYWGCGTQVTVSS

SEQ ID NO: 370 - protein, 124 aa, synthetic construct
QVKLECSGGGLVQAGGSLRLSCAASERTFSRYPVAWFRQAPGAEREFVAVISSTGTSTYYADSVKGRFTISRDNAKVTVYLQMNNLKREDTAVYFCAVNSQRTRLQDPNEYDYWCQGTQVTVSS

SEQ ID NO: 371 - protein, 122 aa, synthetic construct
QVQLVESGGGLVQAGGSLRLSCAASGLTFSRYVMGWFRQAPGKEREFVAAITWGGTPNYADSVKGRFTISRDNSKNTQYLQMNSLKPEDTAVYYCAAGLGWDSRYSQSYNYWGQGTQVTVSS

SEQ ID NO: 372 - protein, 122 aa, synthetic construct
QVQLVESGGGLVQAGGSLRLCCAASGLTFSRYVMGWFRQAPGKEREFVAAITWGGTPNYADSVKGRFTISRDNSKNTQCLQMNSLKPEDTAVYYCAAGLGWDSRYSQSYNYWGQGTQVTVSS

SEQ ID NO: 373 - protein, 122 aa, synthetic construct
QVQLVESGGGLVQAGGSLRLSCCASGLTFSRYVMGWFRQAPGKEREFVAAITWGGTPNYADSVKGRFTISRDNSKNCQYLQMNSLKPEDTAVYYCAAGLGWDSRYSQSYNYWGQGTQVTVSS

SEQ ID NO: 374 - protein, 122 aa, synthetic construct
QVQLVESGGGLVQAGGSLRLSCAASGLTFSRYVMGWFRQCPGKEREFVAAITWGGTPNYADSVKGRFTISRDNSKNTQYLQMNSLKPEDTCVYYCAAGLGWDSRYSQSYNYWGQGTQVTVSS

SEQ ID NO: 375 - protein, 122 aa, synthetic construct
QVQCVESGGGLVQAGGSLRLSCACSGLTFSRYVMGWFRQAPGKEREFVAAITWGGTPNYADSVKGRFTISRDNSKNTQYLQMNSLKPEDTAVYYCAAGLGWDSRYSQSYNYWGQGTQVTVSS

SEQ ID NO: 376 - protein, 122 aa, synthetic construct
QVQCVESGGGLVQAGGSLRLSCAASGLTFSRYVMGWFRQAPGKEREFVAAITWGGTPNYADSVKGRFTISRDNSKNTQYLQMNSLKPEDTAVYYCAAGLGWDSRYSQSYNYCGQGTQVTVSS

SEQ ID NO: 377 - protein, 122 aa, synthetic construct
QVQLCESGGGLVQAGGSLRLSCAASGLTFSRYVMGWFRQAPGKEREFVAAITWGGTPNYADSVKGRFTISRDNSKNTQYLQMNSLKPEDTAVYYCAAGLGWDSRYSQSYNYWGCGTQVTVSS

SEQ ID NO: 378 - protein, 122 aa, synthetic construct
QVQLVCSGGGLVQAGGSLRLSCAASGLTFSRYVMGWFRQAPGKEREFVAAITWGGTPNYADSVKGRFTISRDNSKNTQYLQMNSLKPEDTAVYYCAAGLGWDSRYSQSYNYWCQGTQVTVSS

SEQ ID NO: 379 - protein, 127 aa, synthetic construct
QVKLEESGGGLVQAGGSLRLSCVASEYPSNFYAMSWFRQAPGKEREFVAGVSRDGLTTLYADSVKGRFTMSRDNAKNTVDLQMNSVKAEDTAVYYCAIVITGVWNKVDVNSRSYHYWGQGTQVTVSS

SEQ ID NO: 380 - protein, 127 aa, synthetic construct
QVKLEESGGGLVQAGGSLRLCCVASEYPSNFYAMSWFRQAPGKEREFVAGVSRDGLTTLYADSVKGRFTMSRDNAKNTVCLQMNSVKAEDTAVYYCAIVITGVWNKVDVNSRSYHYWGQGTQVTVSS

SEQ ID NO: 381 - protein, 127 aa, synthetic construct
QVKLEESGGGLVQAGGSLRLSCCASEYPSNFYAMSWFRQAPGKEREFVAGVSRDGLTTLYADSVKGRFTMSRDNAKNCVDLQMNSVKAEDTAVYYCAIVITGVWNKVDVNSRSYHYWGQGTQVTVSS

SEQ ID NO: 382 - protein, 127 aa, synthetic construct
QVKLEESGGGLVQAGGSLRLSCVASEYPSNFYAMSWFRQCPGKEREFVAGVSRDGLTTLYADSVKGRFTMSRDNAKNTVDLQMNSVKAEDTCVYYCAIVITGVWNKVDVNSRSYHYWGQGTQVTVSS

SEQ ID NO: 383 - protein, 127 aa, synthetic construct
QVKCEESGGGLVQAGGSLRLSCVCSEYPSNFYAMSWFRQAPGKEREFVAGVSRDGLTTLYADSVKGRFTMSRDNAKNTVDLQMNSVKAEDTAVYYCAIVITGVWNKVDVNSRSYHYWGQGTQVTVSS

SEQ ID NO: 384 - protein, 127 aa, synthetic construct
QVKCEESGGGLVQAGGSLRLSCVASEYPSNFYAMSWFRQAPGKEREFVAGVSRDGLTTLYADSVKGRFTMSRDNAKNTVDLQMNSVKAEDTAVYYCAIVITGVWNKVDVNSRSYHYCGQGTQVTVSS

SEQ ID NO: 385 - protein, 127 aa, synthetic construct
QVKLCESGGGLVQAGGSLRLSCVASEYPSNFYAMSWFRQAPGKEREFVAGVSRDGLTTLYADSVKGRFTMSRDNAKNTVDLQMNSVKAEDTAVYYCAIVITGVWNKVDVNSRSYHYWGCGTQVTVSS

SEQ ID NO: 386 - protein, 127 aa, synthetic construct
QVKLECSGGGLVQAGGSLRLSCVASEYPSNFYAMSWFRQAPGKEREFVAGVSRDGLTTLYADSVKGRFTMSRDNAKNTVDLQMNSVKAEDTAVYYCAIVITGVWNKVDVNSRSYHYWCQGTQVTVSS

SEQ ID NO: 387 - protein, 124 aa, synthetic construct
QVKLEESGGGLVQAGGSLRLSCEVSGGTVSPTAMGWFRQAPGKEREFVGHITWSRGTTRVASSVKDRFTISRDSAKNTVYLQMNSLKSEDTAVYYCAASTFLRILPEESAYTYWGQGTQVTVSS

SEQ ID NO: 388 - protein, 124 aa, synthetic construct
QVKLEESGGGLVQAGGSLRLCCEVSGGTVSPTAMGWFRQAPGKEREFVGHITWSRGTTRVASSVKDRFTISRDSAKNTVCLQMNSLKSEDTAVYYCAASTFLRILPEESAYTYWGQGTQVTVSS

SEQ ID NO: 389 - protein, 124 aa, synthetic construct
QVKLEESGGGLVQAGGSLRLSCCVSGGTVSPTAMGWFRQAPGKEREFVGHITWSRGTTRVASSVKDRFTISRDSAKNCVYLQMNSLKSEDTAVYYCAASTFLRILPEESAYTYWGQGTQVTVSS

SEQ ID NO: 390 - protein, 124 aa, synthetic construct
QVKLEESGGGLVQAGGSLRLSCEVSGGTVSPTAMGWFRQCPGKEREFVGHITWSRGTTRVASSVKDRFTISRDSAKNTVYLQMNSLKSEDTCVYYCAASTFLRILPEESAYTYWGQGTQVTVSS

SEQ ID NO: 391 - protein, 124 aa, synthetic construct
QVKCEESGGGLVQAGGSLRLSCECSGGTVSPTAMGWFRQAPGKEREFVGHITWSRGTTRVASSVKDRFTISRDSAKNTVYLQMNSLKSEDTAVYYCAASTFLRILPEESAYTYWGQGTQVTVSS

SEQ ID NO: 392 - protein, 124 aa, synthetic construct
QVKCEESGGGLVQAGGSLRLSCEVSGGTVSPTAMGWFRQAPGKEREFVGHITWSRGTTRVASSVKDRFTISRDSAKNTVYLQMNSLKSEDTAVYYCAASTFLRILPEESAYTYCGQGTQVTVSS

SEQ ID NO: 393 - protein, 124 aa, synthetic construct
QVKLCESGGGLVQAGGSLRLSCEVSGGTVSPTAMGWFRQAPGKEREFVGHITWSRGTTRVASSVKDRFTISRDSAKNTVYLQMNSLKSEDTAVYYCAASTFLRILPEESAYTYWGCGTQVTVSS

SEQ ID NO: 394 - protein, 124 aa, synthetic construct
QVKLECSGGGLVQAGGSLRLSCEVSGGTVSPTAMGWFRQAPGKEREFVGHITWSRGTTRVASSVKDRFTISRDSAKNTVYLQMNSLKSEDTAVYYCAASTFLRILPEESAYTYWCQGTQVTVSS

SEQ ID NO: 395 - protein, 124 aa, synthetic construct
QVKLEESGGGLVQAGGSLRLSCAASGRTIDNYAMAWSRQAPGKDREFVATIDWGDGGARYANSVKGRFTISRDNAKGTMYLQMNNLEPEDTAVYSCAMARQSRVNLDVARYDYWGQGTQVTVSS

SEQ ID NO: 396 - protein, 124 aa, synthetic construct
QVKLEESGGGLVQAGGSLRLCCAASGRTIDNYAMAWSRQAPGKDREFVATIDWGDGGARYANSVKGRFTISRDNAKGTMCLQMNNLEPEDTAVYSCAMARQSRVNLDVARYDYWGQGTQVTVSS

SEQ ID NO: 397 - protein, 124 aa, synthetic construct
QVKLEESGGGLVQAGGSLRLSCCASGRTIDNYAMAWSRQAPGKDREFVATIDWGDGGARYANSVKGRFTISRDNAKGCMYLQMNNLEPEDTAVYSCAMARQSRVNLDVARYDYWGQGTQVTVSS

SEQ ID NO: 398 - protein, 124 aa, synthetic construct
QVKLEESGGGLVQAGGSLRLSCAASGRTIDNYAMAWSRQCPGKDREFVATIDWGDGGARYANSVKGRFTISRDNAKGTMYLQMNNLEPEDTCVYSCAMARQSRVNLDVARYDYWGQGTQVTVSS

SEQ ID NO: 399 - protein, 124 aa, synthetic construct
QVKCEESGGGLVQAGGSLRLSCACSGRTIDNYAMAWSRQAPGKDREFVATIDWGDGGARYANSVKGRFTISRDNAKGTMYLQMNNLEPEDTAVYSCAMARQSRVNLDVARYDYWGQGTQVTVSS

SEQ ID NO: 400 - protein, 124 aa, synthetic construct
QVKCEESGGGLVQAGGSLRLSCAASGRTIDNYAMAWSRQAPGKDREFVATIDWGDGGARYANSVKGRFTISRDNAKGTMYLQMNNLEPEDTAVYSCAMARQSRVNLDVARYDYCGQGTQVTVSS

SEQ ID NO: 401 - protein, 124 aa, synthetic construct
QVKLCESGGGLVQAGGSLRLSCAASGRTIDNYAMAWSRQAPGKDREFVATIDWGDGGARYANSVKGRFTISRDNAKGTMYLQMNNLEPEDTAVYSCAMARQSRVNLDVARYDYWGCGTQVTVSS

SEQ ID NO: 402 - protein, 124 aa, synthetic construct
QVKLECSGGGLVQAGGSLRLSCAASGRTIDNYAMAWSRQAPGKDREFVATIDWGDGGARYANSVKGRFTISRDNAKGTMYLQMNNLEPEDTAVYSCAMARQSRVNLDVARYDYWCQGTQVTVSS

SEQ ID NO: 403 - protein, 125 aa, synthetic construct
QVQLVESGGGLVQAGDSLRVSCAASGRDFSDYVMGWFRQAPGKEREFVAAISRNGLTTRYADSVKGRFTISRDNDKNMVYLQMNSLKPEDTAVYYCAVNSAGTYVSPRSREYDYWGQGTQVTVSS

SEQ ID NO: 404 - protein, 125 aa, synthetic construct
QVQLVESGGGLVQAGDSLRVCCAASGRDFSDYVMGWFRQAPGKEREFVAAISRNGLTTRYADSVKGRFTISRDNDKNMVCLQMNSLKPEDTAVYYCAVNSAGTYVSPRSREYDYWGQGTQVTVSS

SEQ ID NO: 405 - protein, 125 aa, synthetic construct
QVQLVESGGGLVQAGDSLRVSCCASGRDFSDYVMGWFRQAPGKEREFVAAISRNGLTTRYADSVKGRFTISRDNDKNCVYLQMNSLKPEDTAVYYCAVNSAGTYVSPRSREYDYWGQGTQVTVSS

SEQ ID NO: 406 - protein, 125 aa, synthetic construct
QVQLVESGGGLVQAGDSLRVSCAASGRDFSDYVMGWFRQCPGKEREFVAAISRNGLTTRYADSVKGRFTISRDNDKNMVYLQMNSLKPEDTCVYYCAVNSAGTYVSPRSREYDYWGQGTQVTVSS

SEQ ID NO: 407 - protein, 125 aa, synthetic construct
QVQCVESGGGLVQAGDSLRVSCACSGRDFSDYVMGWFRQAPGKEREFVAAISRNGLTTRYADSVKGRFTISRDNDKNMVYLQMNSLKPEDTAVYYCAVNSAGTYVSPRSREYDYWGQGTQVTVSS

SEQ ID NO: 408 - protein, 125 aa, synthetic construct
QVQCVESGGGLVQAGDSLRVSCAASGRDFSDYVMGWFRQAPGKEREFVAAISRNGLTTRYADSVKGRFTISRDNDKNMVYLQMNSLKPEDTAVYYCAVNSAGTYVSPRSREYDYCGQGTQVTVSS

SEQ ID NO: 409 - protein, 125 aa, synthetic construct
QVQLCESGGGLVQAGDSLRVSCAASGRDFSDYVMGWFRQAPGKEREFVAAISRNGLTTRYADSVKGRFTISRDNDKNMVYLQMNSLKPEDTAVYYCAVNSAGTYVSPRSREYDYWGCGTQVTVSS

SEQ ID NO: 410 - protein, 125 aa, synthetic construct
QVQLVCSGGGLVQAGDSLRVSCAASGRDFSDYVMGWFRQAPGKEREFVAAISRNGLTTRYADSVKGRFTISRDNDKNMVYLQMNSLKPEDTAVYYCAVNSAGTYVSPRSREYDYWCQGTQVTVSS

SEQ ID NO: 411 - protein, 122 aa, synthetic construct
DVQLQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSS

SEQ ID NO: 412 - protein, 122 aa, synthetic construct
DVQLQASGGGLVQAGGSLRLCCAASGFKITHYTMGWFRQAPGKEREFVSRITWGGDNTFYSNSVKGRFTISRDNAKNTVCLQMNSLKPEDTADYYCAAGSTSTATPLRVDYWGKGTQVTVSS

SEQ ID NO: 413 - protein, 122 aa, synthetic construct
DVQLQASGGGLVQAGGSLRLSCCASGFKITHYTMGWFRQAPGKEREFVSRITWGGDNTFYSNSVKGRFTISRDNAKNCVYLQMNSLKPEDTADYYCAAGSTSTATPLRVDYWGKGTQVTVSS

SEQ ID NO: 414 - protein, 122 aa, synthetic construct
DVQLQASGGGLVQAGGSLRLSCAASGFKITHYTMGWFRQCPGKEREFVSRITWGGDNTFYSNSVKGRFTISRDNAKNTVYLQMNSLKPEDTCDYYCAAGSTSTATPLRVDYWGKGTQVTVSS

SEQ ID NO: 415 - protein, 122 aa, synthetic construct
DVQCQASGGGLVQAGGSLRLSCACSGFKITHYTMGWFRQAPGKEREFVSRITWGGDNTFYSNSVKGRFTISRDNAKNTVYLQMNSLKPEDTADYYCAAGSTSTATPLRVDYWGKGTQVTVSS

SEQ ID NO: 416 - protein, 122 aa, synthetic construct
DVQCQASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYCGKGTQVTVSS

SEQ ID NO: 417 - protein, 122 aa, synthetic construct
DVQLCASGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWGCGTQVTVSS

SEQ ID NO: 418 - protein, 122 aa, synthetic construct
DVQLQCSGGGLVQAGGSLRLSCAASGFKITHYTMGWFRQAPGKEREFVSRITWGGDNTFYSNSVKGRFTISRDNAKNTVYLQMNSLKPEDTADYYCAAGSTSTATPLRVDYWCKGTQVTVSS

SEQ ID NO: 419 - protein, 119 aa, synthetic construct
QVQLVQSGGGLVKPGGSLRLSCAASGFTFSDYYMSWVRQAPGKGLEWVSFIYSGGSTYYADSVKGRFTISRDNSKNTLYLQMNSLRAEDTAVYYCARESRVGGGAFDIWGQGTMVTVSS

SEQ ID NO: 420 - protein, 119 aa, synthetic construct
QVQLVQSGGGLVKPGGSLRLCCAASGFTFSDYYMSWVRQAPGKGLEWVSFIYSGGSTYYADSVKGRFTISRDNSKNTLCLQMNSLRAEDTAVYYCARESRVGGGAFDIWGQGTMVTVSS

SEQ ID NO: 421 - protein, 119 aa, synthetic construct
QVQLVQSGGGLVKPGGSLRLSCCASGFTFSDYYMSWVRQAPGKGLEWVSFIYSGGSTYYADSVKGRFTISRDNSKNCLYLQMNSLRAEDTAVYYCARESRVGGGAFDIWGQGTMVTVSS

SEQ ID NO: 422 - protein, 119 aa, synthetic construct
QVQLVQSGGGLVKPGGSLRLSCAASGFTFSDYYMSWVRQCPGKGLEWVSFIYSGGSTYYADSVKGRFTISRDNSKNTLYLQMNSLRAEDTCVYYCARESRVGGGAFDIWGQGTMVTVSS

SEQ ID NO: 423 - protein, 119 aa, synthetic construct
QVQCVQSGGGLVKPGGSLRLSCACSGFTFSDYYMSWVRQAPGKGLEWVSFIYSGGSTYYADSVKGRFTISRDNSKNTLYLQMNSLRAEDTAVYYCARESRVGGGAFDIWGQGTMVTVSS

SEQ ID NO: 424 - protein, 119 aa, synthetic construct
QVQCVQSGGGLVKPGGSLRLSCAASGFTFSDYYMSWVRQAPGKGLEWVSFIYSGGSTYYADSVKGRFTISRDNSKNTLYLQMNSLRAEDTAVYYCARESRVGGGAFDICGQGTMVTVSS

SEQ ID NO: 425 - protein, 119 aa, synthetic construct
QVQLCQSGGGLVKPGGSLRLSCAASGFTFSDYYMSWVRQAPGKGLEWVSFIYSGGSTYYADSVKGRFTISRDNSKNTLYLQMNSLRAEDTAVYYCARESRVGGGAFDIWGCGTMVTVSS

SEQ ID NO: 426 - protein, 119 aa, synthetic construct
QVQLVCSGGGLVKPGGSLRLSCAASGFTFSDYYMSWVRQAPGKGLEWVSFIYSGGSTYYADSVKGRFTISRDNSKNTLYLQMNSLRAEDTAVYYCARESRVGGGAFDIWCQGTMVTVSS

SEQ ID NO: 427 - protein, 119 aa, synthetic construct
DVQLVQSGGGLVKPGGSLRLSCTASGFPFSNAWMSWVRQAPGKGLEWVGRITSKTDGGTTDYVAPVKGRFTISRDDSKNTLYLQMNSLKTEDTAVYYCTTDQANAFDIWGQGTMVTVSS

SEQ ID NO: 428 - protein, 119 aa, synthetic construct
DVQLVQSGGGLVKPGGSLRLCCTASGFPFSNAWMSWVRQAPGKGLEWVGRITSKTDGGTTDYVAPVKGRFTISRDDSKNTLCLQMNSLKTEDTAVYYCTTDQANAFDIWGQGTMVTVSS

SEQ ID NO: 429 - protein, 119 aa, synthetic construct
DVQLVQSGGGLVKPGGSLRLSCCASGFPFSNAWMSWVRQAPGKGLEWVGRITSKTDGGTTDYVAPVKGRFTISRDDSKNCLYLQMNSLKTEDTAVYYCTTDQANAFDIWGQGTMVTVSS

SEQ ID NO: 430 - protein, 119 aa, synthetic construct
DVQLVQSGGGLVKPGGSLRLSCTASGFPFSNAWMSWVRQCPGKGLEWVGRITSKTDGGTTDYVAPVKGRFTISRDDSKNTLYLQMNSLKTEDTCVYYCTTDQANAFDIWGQGTMVTVSS

SEQ ID NO: 431 - protein, 119 aa, synthetic construct
DVQCVQSGGGLVKPGGSLRLSCTCSGFPFSNAWMSWVRQAPGKGLEWVGRITSKTDGGTTDYVAPVKGRFTISRDDSKNTLYLQMNSLKTEDTAVYYCTTDQANAFDIWGQGTMVTVSS

SEQ ID NO: 432 - protein, 119 aa, synthetic construct
DVQCVQSGGGLVKPGGSLRLSCTASGFPFSNAWMSWVRQAPGKGLEWVGRITSKTDGGTTDYVAPVKGRFTISRDDSKNTLYLQMNSLKTEDTAVYYCTTDQANAFDICGQGTMVTVSS

SEQ ID NO: 433 - protein, 119 aa, synthetic construct
DVQLCQSGGGLVKPGGSLRLSCTASGFPFSNAWMSWVRQAPGKGLEWVGRITSKTDGGTTDYVAPVKGRFTISRDDSKNTLYLQMNSLKTEDTAVYYCTTDQANAFDIWGCGTMVTVSS

SEQ ID NO: 434 - protein, 119 aa, synthetic construct
DVQLVCSGGGLVKPGGSLRLSCTASGFPFSNAWMSWVRQAPGKGLEWVGRITSKTDGGTTDYVAPVKGRFTISRDDSKNTLYLQMNSLKTEDTAVYYCTTDQANAFDIWCQGTMVTVSS

SEQ ID NO: 435 - protein, 122 aa, synthetic construct
QLQLQESGGGLVQPGGSLRLSCAASGFTFSSYAMSWVRQAPGKGLEWVSAISGSGGSTYYADSVKGRFTISRDNSKNSLYLQMNSLGAEDTAVYYCARSWSGSSYGGDLDSWGQGTLVTVSS

SEQ ID NO: 436 - protein, 122 aa, synthetic construct
QLQLQESGGGLVQPGGSLRLCCAASGFTFSSYAMSWVRQAPGKGLEWVSAISGSGGSTYYADSVKGRFTISRDNSKNSLCLQMNSLGAEDTAVYYCARSWSGSSYGGDLDSWGQGTLVTVSS

SEQ ID NO: 437 - protein, 122 aa, synthetic construct
QLQLQESGGGLVQPGGSLRLSCCASGFTFSSYAMSWVRQAPGKGLEWVSAISGSGGSTYYADSVKGRFTISRDNSKNCLYLQMNSLGAEDTAVYYCARSWSGSSYGGDLDSWGQGTLVTVSS

SEQ ID NO: 438 - protein, 122 aa, synthetic construct
QLQLQESGGGLVQPGGSLRLSCAASGFTFSSYAMSWVRQCPGKGLEWVSAISGSGGSTYYADSVKGRFTISRDNSKNSLYLQMNSLGAEDTCVYYCARSWSGSSYGGDLDSWGQGTLVTVSS

SEQ ID NO: 439 - protein, 122 aa, synthetic construct
QLQCQESGGGLVQPGGSLRLSCACSGFTFSSYAMSWVRQAPGKGLEWVSAISGSGGSTYYADSVKGRFTISRDNSKNSLYLQMNSLGAEDTAVYYCARSWSGSSYGGDLDSWGQGTLVTVSS

SEQ ID NO: 440 - protein, 122 aa, synthetic construct
QLQCQESGGGLVQPGGSLRLSCAASGFTFSSYAMSWVRQAPGKGLEWVSAISGSGGSTYYADSVKGRFTISRDNSKNSLYLQMNSLGAEDTAVYYCARSWSGSSYGGDLDSCGQGTLVTVSS

SEQ ID NO: 441 - protein, 122 aa, synthetic construct
QLQLCESGGGLVQPGGSLRLSCAASGFTFSSYAMSWVRQAPGKGLEWVSAISGSGGSTYYADSVKGRFTISRDNSKNSLYLQMNSLGAEDTAVYYCARSWSGSSYGGDLDSWGCGTLVTVSS

SEQ ID NO: 442 - protein, 122 aa, synthetic construct
QLQLQCSGGGLVQPGGSLRLSCAASGFTFSSYAMSWVRQAPGKGLEWVSAISGSGGSTYYADSVKGRFTISRDNSKNSLYLQMNSLGAEDTAVYYCARSWSGSSYGGDLDSWCQGTLVTVSS

SEQ ID NO: 443 - protein, 119 aa, synthetic construct
QVQLVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGQGTLVTVSS

SEQ ID NO: 444 - protein, 119 aa, synthetic construct
QVQLVESGGGLVKPGGSLRLCCAASGFTFSNAWMTWVRQAPGKGLEWVGRIKTKTDGGTTDYAAPVKGRFTISRDDSKNTLCLQMNSLKTEDTAVYYCTTDRDHSSGSWGQGTLVTVSS

SEQ ID NO: 445 - protein, 119 aa, synthetic construct
QVQLVESGGGLVKPGGSLRLSCCASGFTFSNAWMTWVRQAPGKGLEWVGRIKTKTDGGTTDYAAPVKGRFTISRDDSKNCLYLQMNSLKTEDTAVYYCTTDRDHSSGSWGQGTLVTVSS

SEQ ID NO: 446 - protein, 119 aa, synthetic construct
QVQLVESGGGLVKPGGSLRLSCAASGFTFSNAWMTWVRQCPGKGLEWVGRIKTKTDGGTTDYAAPVKGRFTISRDDSKNTLYLQMNSLKTEDTCVYYCTTDRDHSSGSWGQGTLVTVSS

SEQ ID NO: 447 - protein, 119 aa, synthetic construct
QVQCVESGGGLVKPGGSLRLSCACSGFTFSNAWMTWVRQAPGKGLEWVGRIKTKTDGGTTDYAAPVKGRFTISRDDSKNTLYLQMNSLKTEDTAVYYCTTDRDHSSGSWGQGTLVTVSS

SEQ ID NO: 448 - protein, 119 aa, synthetic construct
QVQCVESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSCGQGTLVTVSS

SEQ ID NO: 449 - protein, 119 aa, synthetic construct
QVQLCESGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWGCGTLVTVSS

SEQ ID NO: 450 - protein, 119 aa, synthetic construct
QVQLVCSGGGLVKPGGSLRLSCAASGFTFSNAWMTWVRQAPGKGLEWVGRIKTKTDGGTTDYAAPVKGRFTISRDDSKNTLYLQMNSLKTEDTAVYYCTTDRDHSSGSWCQGTLVTVSS

SEQ ID NO: 451 - protein, 123 aa, synthetic construct
QLQLQESGGGVVQPGRSLRLSCAASGFTFSSYAMSWVRQAPGKGLEWVSAISGSGGSTYYADSVKGRFTISRDNSKNTLYLQMNSLRAEDTAVYYCAKDGKGGSSGYDHPDYWGQGTLVTVSS

SEQ ID NO: 452 - protein, 123 aa, synthetic construct
QLQLQESGGGVVQPGRSLRLCCAASGFTFSSYAMSWVRQAPGKGLEWVSAISGSGGSTYYADSVKGRFTISRDNSKNTLCLQMNSLRAEDTAVYYCAKDGKGGSSGYDHPDYWGQGTLVTVSS

SEQ ID NO: 453 - protein, 123 aa, synthetic construct
QLQLQESGGGVVQPGRSLRLSCCASGFTFSSYAMSWVRQAPGKGLEWVSAISGSGGSTYYADSVKGRFTISRDNSKNCLYLQMNSLRAEDTAVYYCAKDGKGGSSGYDHPDYWGQGTLVTVSS

SEQ ID NO: 454 - protein, 123 aa, synthetic construct
QLQLQESGGGVVQPGRSLRLSCAASGFTFSSYAMSWVRQCPGKGLEWVSAISGSGGSTYYADSVKGRFTISRDNSKNTLYLQMNSLRAEDTCVYYCAKDGKGGSSGYDHPDYWGQGTLVTVSS

SEQ ID NO: 455 - protein, 123 aa, synthetic construct
QLQCQESGGGVVQPGRSLRLSCACSGFTFSSYAMSWVRQAPGKGLEWVSAISGSGGSTYYADSVKGRFTISRDNSKNTLYLQMNSLRAEDTAVYYCAKDGKGGSSGYDHPDYWGQGTLVTVSS

SEQ ID NO: 456 - protein, 123 aa, synthetic construct
QLQCQESGGGVVQPGRSLRLSCAASGFTFSSYAMSWVRQAPGKGLEWVSAISGSGGSTYYADSVKGRFTISRDNSKNTLYLQMNSLRAEDTAVYYCAKDGKGGSSGYDHPDYCGQGTLVTVSS

SEQ ID NO: 457 - protein, 123 aa, synthetic construct
QLQLCESGGGVVQPGRSLRLSCAASGFTFSSYAMSWVRQAPGKGLEWVSAISGSGGSTYYADSVKGRFTISRDNSKNTLYLQMNSLRAEDTAVYYCAKDGKGGSSGYDHPDYWGCGTLVTVSS

SEQ ID NO: 458 - protein, 123 aa, synthetic construct
QLQLQCSGGGVVQPGRSLRLSCAASGFTFSSYAMSWVRQAPGKGLEWVSAISGSGGSTYYADSVKGRFTISRDNSKNTLYLQMNSLRAEDTAVYYCAKDGKGGSSGYDHPDYWCQGTLVTVSS

SEQ ID NO: 459 - protein, 118 aa, synthetic construct
QMQLVQSGGGVVQPGGSLRLSCAASGFTVSSSRMSWFRQAPGMGLEWVSVIYSGGSTYYADSVRGRFSISRDNSKNTLYLQMNSLRAEDTALYYCAREREGAVTREDWGQGTLVTVSS

SEQ ID NO: 460 - protein, 118 aa, synthetic construct
QMQLVQSGGGVVQPGGSLRLCCAASGFTVSSSRMSWFRQAPGMGLEWVSVIYSGGSTYYADSVRGRFSISRDNSKNTLCLQMNSLRAEDTALYYCAREREGAVTREDWGQGTLVTVSS

SEQ ID NO: 461 - protein, 118 aa, synthetic construct
QMQLVQSGGGVVQPGGSLRLSCCASGFTVSSSRMSWFRQAPGMGLEWVSVIYSGGSTYYADSVRGRFSISRDNSKNCLYLQMNSLRAEDTALYYCAREREGAVTREDWGQGTLVTVSS

SEQ ID NO: 462 - protein, 118 aa, synthetic construct
QMQLVQSGGGVVQPGGSLRLSCAASGFTVSSSRMSWFRQCPGMGLEWVSVIYSGGSTYYADSVRGRFSISRDNSKNTLYLQMNSLRAEDTCLYYCAREREGAVTREDWGQGTLVTVSS

SEQ ID NO: 463 - protein, 118 aa, synthetic construct
QMQCVQSGGGVVQPGGSLRLSCACSGFTVSSSRMSWFRQAPGMGLEWVSVIYSGGSTYYADSVRGRFSISRDNSKNTLYLQMNSLRAEDTALYYCAREREGAVTREDWGQGTLVTVSS

SEQ ID NO: 464 - protein, 118 aa, synthetic construct
QMQCVQSGGGVVQPGGSLRLSCAASGFTVSSSRMSWFRQAPGMGLEWVSVIYSGGSTYYADSVRGRFSISRDNSKNTLYLQMNSLRAEDTALYYCAREREGAVTREDCGQGTLVTVSS

SEQ ID NO: 465 - protein, 118 aa, synthetic construct
QMQLCQSGGGVVQPGGSLRLSCAASGFTVSSSRMSWFRQAPGMGLEWVSVIYSGGSTYYADSVRGRFSISRDNSKNTLYLQMNSLRAEDTALYYCAREREGAVTREDWGCGTLVTVSS

SEQ ID NO: 466 - protein, 118 aa, synthetic construct
QMQLVCSGGGVVQPGGSLRLSCAASGFTVSSSRMSWFRQAPGMGLEWVSVIYSGGSTYYADSVRGRFSISRDNSKNTLYLQMNSLRAEDTALYYCAREREGAVTREDWCQGTLVTVSS

SEQ ID NO: 467 - protein, 123 aa, synthetic construct
QLQLQESGGGLVQPGGSLRLSCAASGFTFSSYAMSWFRQAPGKGLEWVGFIRSKAYGGTTEYAASVKGRFTISRDDSKSIAYLQMNSLRAEDTAMYYCARRAKDGYNSPEDYWGQGTLVTVSS

SEQ ID NO: 468 - protein, 123 aa, synthetic construct
QLQLQESGGGLVQPGGSLRLCCAASGFTFSSYAMSWFRQAPGKGLEWVGFIRSKAYGGTTEYAASVKGRFTISRDDSKSIACLQMNSLRAEDTAMYYCARRAKDGYNSPEDYWGQGTLVTVSS

SEQ ID NO: 469 - protein, 123 aa, synthetic construct
QLQLQESGGGLVQPGGSLRLSCCASGFTFSSYAMSWFRQAPGKGLEWVGFIRSKAYGGTTEYAASVKGRFTISRDDSKSCAYLQMNSLRAEDTAMYYCARRAKDGYNSPEDYWGQGTLVTVSS

SEQ ID NO: 470 - protein, 123 aa, synthetic construct
QLQLQESGGGLVQPGGSLRLSCAASGFTFSSYAMSWFRQCPGKGLEWVGFIRSKAYGGTTEYAASVKGRFTISRDDSKSIAYLQMNSLRAEDTCMYYCARRAKDGYNSPEDYWGQGTLVTVSS

SEQ ID NO: 471 - protein, 123 aa, synthetic construct
QLQCQESGGGLVQPGGSLRLSCACSGFTFSSYAMSWFRQAPGKGLEWVGFIRSKAYGGTTEYAASVKGRFTISRDDSKSIAYLQMNSLRAEDTAMYYCARRAKDGYNSPEDYWGQGTLVTVSS

SEQ ID NO: 472 - protein, 123 aa, synthetic construct
QLQCQESGGGLVQPGGSLRLSCAASGFTFSSYAMSWFRQAPGKGLEWVGFIRSKAYGGTTEYAASVKGRFTISRDDSKSIAYLQMNSLRAEDTAMYYCARRAKDGYNSPEDYCGQGTLVTVSS

SEQ ID NO: 473 - protein, 123 aa, synthetic construct
QLQLCESGGGLVQPGGSLRLSCAASGFTFSSYAMSWFRQAPGKGLEWVGFIRSKAYGGTTEYAASVKGRFTISRDDSKSIAYLQMNSLRAEDTAMYYCARRAKDGYNSPEDYWGCGTLVTVSS

SEQ ID NO: 474 - protein, 123 aa, synthetic construct
QLQLQCSGGGLVQPGGSLRLSCAASGFTFSSYAMSWFRQAPGKGLEWVGFIRSKAYGGTTEYAASVKGRFTISRDDSKSIAYLQMNSLRAEDTAMYYCARRAKDGYNSPEDYWCQGTLVTVSS

SEQ ID NO: 475 - protein, 119 aa, synthetic construct
EVQLVESGGTLVQPGGSLRLSCAASGFTFINYAMSWVRQAPDKGLDWVSTISNNGGATYYADSVKGRFTISRDNSNNTLYLQMNSLRPDDTAVYYCAKGPINTGRYGDWGQGTLVTVSS

SEQ ID NO: 476 - protein, 119 aa, synthetic construct
EVQLVESGGTLVQPGGSLRLCCAASGFTFINYAMSWVRQAPDKGLDWVSTISNNGGATYYADSVKGRFTISRDNSNNTLCLQMNSLRPDDTAVYYCAKGPINTGRYGDWGQGTLVTVSS

SEQ ID NO: 477 - protein, 119 aa, synthetic construct
EVQLVESGGTLVQPGGSLRLSCCASGFTFINYAMSWVRQAPDKGLDWVSTISNNGGATYYADSVKGRFTISRDNSNNCLYLQMNSLRPDDTAVYYCAKGPINTGRYGDWGQGTLVTVSS

SEQ ID NO: 478 - protein, 119 aa, synthetic construct
EVQLVESGGTLVQPGGSLRLSCAASGFTFINYAMSWVRQCPDKGLDWVSTISNNGGATYYADSVKGRFTISRDNSNNTLYLQMNSLRPDDTCVYYCAKGPINTGRYGDWGQGTLVTVSS

SEQ ID NO: 479 - protein, 119 aa, synthetic construct
EVQCVESGGTLVQPGGSLRLSCACSGFTFINYAMSWVRQAPDKGLDWVSTISNNGGATYYADSVKGRFTISRDNSNNTLYLQMNSLRPDDTAVYYCAKGPINTGRYGDWGQGTLVTVSS

SEQ ID NO: 480 - protein, 119 aa, synthetic construct
EVQCVESGGTLVQPGGSLRLSCAASGFTFINYAMSWVRQAPDKGLDWVSTISNNGGATYYADSVKGRFTISRDNSNNTLYLQMNSLRPDDTAVYYCAKGPINTGRYGDCGQGTLVTVSS

SEQ ID NO: 481 - protein, 119 aa, synthetic construct
EVQLCESGGTLVQPGGSLRLSCAASGFTFINYAMSWVRQAPDKGLDWVSTISNNGGATYYADSVKGRFTISRDNSNNTLYLQMNSLRPDDTAVYYCAKGPINTGRYGDWGCGTLVTVSS

SEQ ID NO: 482 - protein, 119 aa, synthetic construct
EVQLVCSGGTLVQPGGSLRLSCAASGFTFINYAMSWVRQAPDKGLDWVSTISNNGGATYYADSVKGRFTISRDNSNNTLYLQMNSLRPDDTAVYYCAKGPINTGRYGDWCQGTLVTVSS

SEQ ID NO: 483 - protein, 123 aa, synthetic construct
QVQLVQSGGGLVQPGRSLRLSCAASGFAFSSYAMSWVRQAPGKGLEWVSAISGGGDHTYYADSVKGRFTISRDNSKNTLYLQMNSLRAEDTAVYYCAKEGMVRGVSSAPFDYWGQGTLVTVSS

SEQ ID NO: 484 - protein, 123 aa, synthetic construct
QVQLVQSGGGLVQPGRSLRLCCAASGFAFSSYAMSWVRQAPGKGLEWVSAISGGGDHTYYADSVKGRFTISRDNSKNTLCLQMNSLRAEDTAVYYCAKEGMVRGVSSAPFDYWGQGTLVTVSS

SEQ ID NO: 485 - protein, 123 aa, synthetic construct
QVQLVQSGGGLVQPGRSLRLSCCASGFAFSSYAMSWVRQAPGKGLEWVSAISGGGDHTYYADSVKGRFTISRDNSKNCLYLQMNSLRAEDTAVYYCAKEGMVRGVSSAPFDYWGQGTLVTVSS

SEQ ID NO: 486 - protein, 123 aa, synthetic construct
QVQLVQSGGGLVQPGRSLRLSCAASGFAFSSYAMSWVRQCPGKGLEWVSAISGGGDHTYYADSVKGRFTISRDNSKNTLYLQMNSLRAEDTCVYYCAKEGMVRGVSSAPFDYWGQGTLVTVSS

SEQ ID NO: 487 - protein, 123 aa, synthetic construct
QVQCVQSGGGLVQPGRSLRLSCACSGFAFSSYAMSWVRQAPGKGLEWVSAISGGGDHTYYADSVKGRFTISRDNSKNTLYLQMNSLRAEDTAVYYCAKEGMVRGVSSAPFDYWGQGTLVTVSS

SEQ ID NO: 488 - protein, 123 aa, synthetic construct
QVQCVQSGGGLVQPGRSLRLSCAASGFAFSSYAMSWVRQAPGKGLEWVSAISGGGDHTYYADSVKGRFTISRDNSKNTLYLQMNSLRAEDTAVYYCAKEGMVRGVSSAPFDYCGQGTLVTVSS

SEQ ID NO: 489 - protein, 123 aa, synthetic construct
QVQLCQSGGGLVQPGRSLRLSCAASGFAFSSYAMSWVRQAPGKGLEWVSAISGGGDHTYYADSVKGRFTISRDNSKNTLYLQMNSLRAEDTAVYYCAKEGMVRGVSSAPFDYWGCGTLVTVSS

SEQ ID NO: 490 - protein, 123 aa, synthetic construct
QVQLVCSGGGLVQPGRSLRLSCAASGFAFSSYAMSWVRQAPGKGLEWVSAISGGGDHTYYADSVKGRFTISRDNSKNTLYLQMNSLRAEDTAVYYCAKEGMVRGVSSAPFDYWCQGTLVTVSS

SEQ ID NO: 491 - protein, 124 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEPRSVSGLRGVVDSWGRGTLVTVSS

SEQ ID NO: 492 - protein, 124 aa, synthetic construct
EVQLVESGGGLVQPGGSLRLCCAASGFTFSSYAMSWVRQAPGKGLEWVSAISGSGGSTYYADSVKGRFTISRDNSKNTLCLQMNSLRAEDTAVYYCAKDEPRSVSGLRGVVDSWGRGTLVTVSS

SEQ ID NO: 493 - protein, 124 aa, synthetic construct
EVQLVESGGGLVQPGGSLRLSCCASGFTFSSYAMSWVRQAPGKGLEWVSAISGSGGSTYYADSVKGRFTISRDNSKNCLYLQMNSLRAEDTAVYYCAKDEPRSVSGLRGVVDSWGRGTLVTVSS

SEQ ID NO: 494 - protein, 124 aa, synthetic construct
EVQLVESGGGLVQPGGSLRLSCAASGFTFSSYAMSWVRQCPGKGLEWVSAISGSGGSTYYADSVKGRFTISRDNSKNTLYLQMNSLRAEDTCVYYCAKDEPRSVSGLRGVVDSWGRGTLVTVSS

SEQ ID NO: 495 - protein, 124 aa, synthetic construct
EVQCVESGGGLVQPGGSLRLSCACSGFTFSSYAMSWVRQAPGKGLEWVSAISGSGGSTYYADSVKGRFTISRDNSKNTLYLQMNSLRAEDTAVYYCAKDEPRSVSGLRGVVDSWGRGTLVTVSS

SEQ ID NO: 496 - protein, 124 aa, synthetic construct
EVQCVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEPRSVSGLRGVVDSCGRGTLVTVSS

SEQ ID NO: 497 - protein, 124 aa, synthetic construct
EVQLCESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEPRSVSGLRGVVDSWGCGTLVTVSS

SEQ ID NO: 498 - protein, 124 aa, synthetic construct
EVQLVCSGGGLVQPGGSLRLSCAASGFTFSSYAMSWVRQAPGKGLEWVSAISGSGGSTYYADSVKGRFTISRDNSKNTLYLQMNSLRAEDTAVYYCAKDEPRSVSGLRGVVDSWCRGTLVTVSS

SEQ ID NO: 499 - protein, 121 aa, synthetic construct
EVQLVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGQGTMVTVSS

SEQ ID NO: 500 - protein, 121 aa, synthetic construct
EVQLVQSGGGLVQPGRSLRLCCAASGFTFDDYAMHWVRQAPGKGLEWVSGISGSGASTYYADSVKGRFTISRDNSKNTLCLQMNSLRAGDTALYYCARQSITGPTGAFDVWGQGTMVTVSS

SEQ ID NO: 501 - protein, 121 aa, synthetic construct
EVQLVQSGGGLVQPGRSLRLSCCASGFTFDDYAMHWVRQAPGKGLEWVSGISGSGASTYYADSVKGRFTISRDNSKNCLYLQMNSLRAGDTALYYCARQSITGPTGAFDVWGQGTMVTVSS

SEQ ID NO: 502 - protein, 121 aa, synthetic construct
EVQLVQSGGGLVQPGRSLRLSCAASGFTFDDYAMHWVRQCPGKGLEWVSGISGSGASTYYADSVKGRFTISRDNSKNTLCLQMNSLRAGDTALYYCARQSITGPTGAFDVWGQGTMVTVSS

SEQ ID NO: 503 - protein, 121 aa, synthetic construct
EVQCVQSGGGLVQPGRSLRLSCACSGFTFDDYAMHWVRQAPGKGLEWVSGISGSGASTYYADSVKGRFTISRDNSKNTLYLQMNSLRAGDTALYYCARQSITGPTGAFDVWGQGTMVTVSS

SEQ ID NO: 504 - protein, 121 aa, synthetic construct
EVQCVQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVCGQGTMVTVSS

SEQ ID NO: 505 - protein, 121 aa, synthetic construct
EVQLCQSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWGCGTMVTVSS

SEQ ID NO: 506 - protein, 121 aa, synthetic construct
EVQLVCSGGGLVQPGRSLRLSCAASGFTFDDYAMHWVRQAPGKGLEWVSGISGSGASTYYADSVKGRFTISRDNSKNTLYLQMNSLRAGDTALYYCARQSITGPTGAFDVWCQGTMVTVSS

SEQ ID NO: 507 - protein, 113 aa, synthetic construct
QVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCGTDMEVWGKGTTVTVSS

SEQ ID NO: 508 - protein, 113 aa, synthetic construct
QVQLQESGGGLVQPGGSLRLCCAASGFTFSSYAMSWVRQAPGKGLEWVSAISGSGGSTYYADSVKGRFTISRDNSKNTLCLQMNSLRAEDTAVYYCGTDMEVWGKGTTVTVSS

SEQ ID NO: 509 - protein, 113 aa, synthetic construct
QVQLQESGGGLVQPGGSLRLSCCASGFTFSSYAMSWVRQAPGKGLEWVSAISGSGGSTYYADSVKGRFTISRDNSKNCLYLQMNSLRAEDTAVYYCGTDMEVWGKGTTVTVSS

SEQ ID NO: 510 - protein, 113 aa, synthetic construct
QVQLQESGGGLVQPGGSLRLSCAASGFTFSSYAMSWVRQCPGKGLEWVSAISGSGGSTYYADSVKGRFTISRDNSKNTLYLQMNSLRAEDTCVYYCGTDMEVWGKGTTVTVSS

SEQ ID NO: 511 - protein, 113 aa, synthetic construct
QVQCQESGGGLVQPGGSLRLSCACSGFTFSSYAMSWVRQAPGKGLEWVSAISGSGGSTYYADSVKGRFTISRDNSKNTLYLQMNSLRAEDTAVYYCGTDMEVWGKGTTVTVSS

SEQ ID NO: 512 - protein, 113 aa, synthetic construct
QVQCQESGGGLVQPGGSLRLSCAASGFTFSSYAMSWVRQAPGKGLEWVSAISGSGGSTYYADSVKGRFTISRDNSKNTLYLQMNSLRAEDTAVYYCGTDMEVCGKGTTVTVSS

SEQ ID NO: 513 - protein, 113 aa, synthetic construct
QVQLCESGGGLVQPGGSLRLSCAASGFTFSSYAMSWVRQAPGKGLEWVSAISGSGGSTYYADSVKGRFTISRDNSKNTLYLQMNSLRAEDTAVYYCGTDMEVWGCGTTVTVSS

SEQ ID NO: 514 - protein, 113 aa, synthetic construct
QVQLQCSGGGLVQPGGSLRLSCAASGFTFSSYAMSWVRQAPGKGLEWVSAISGSGGSTYYADSVKGRFTISRDNSKNTLYLQMNSLRAEDTAVYYCGTDMEVWCKGTTVTVSS

SEQ ID NO: 515 - protein, 107 aa, synthetic construct
DIQMTQSPSSLSASVGDRVTITCRASQSISTYLNWYQQKPGKAPKLLIFAASTLQSGVPSRFSGSGSGTDFTLTISNLQPEDFATYYCQQSYSTPRTFGHGTKVTVL

SEQ ID NO: 516 - protein, 107 aa, synthetic construct
DIQMTQSPSSLSASVGDRVTICCRASQSISTYLNWYQQKPGKAPKLLIFAASTLQSGVPSRFSGSGSGTDFCLTISNLQPEDFATYYCQQSYSTPRTFGHGTKVTVL

SEQ ID NO: 517 - protein, 107 aa, synthetic construct
DIQMTQSPSSLSASVGDRVTITCCASQSISTYLNWYQQKPGKAPKLLIFAASTLQSGVPSRFSGSGSGTCFTLTISNLQPEDFATYYCQQSYSTPRTFGHGTKVTVL

SEQ ID NO: 518 - protein, 107 aa, synthetic construct
DIQMTQSPSSLSASVGDRVTITCRASQSISTYLNWYQQCPGKAPKLLIFAASTLQSGVPSRFSGSGSGTDFTLTISNLQPEDFCTYYCQQSYSTPRTFGHGTKVTVL

SEQ ID NO: 519 - protein, 107 aa, synthetic construct
DIQCTQSPSSLSASVGDRVTITCRCSQSISTYLNWYQQKPGKAPKLLIFAASTLQSGVPSRFSGSGSGTDFTLTISNLQPEDFATYYCQQSYSTPRTFGHGTKVTVL

SEQ ID NO: 520 - protein, 107 aa, synthetic construct
DIQCTQSPSSLSASVGDRVTITCRASQSISTYLNWYQQKPGKAPKLLIFAASTLQSGVPSRFSGSGSGTDFTLTISNLQPEDFATYYCQQSYSTPRTCGHGTKVTVL

SEQ ID NO: 521 - protein, 107 aa, synthetic construct
DIQMCQSPSSLSASVGDRVTITCRASQSISTYLNWYQQKPGKAPKLLIFAASTLQSGVPSRFSGSGSGTDFTLTISNLQPEDFATYYCQQSYSTPRTFGCGTKVTVL

SEQ ID NO: 522 - protein, 107 aa, synthetic construct
DIQMTCSPSSLSASVGDRVTITCRASQSISTYLNWYQQKPGKAPKLLIFAASTLQSGVPSRFSGSGSGTDFTLTISNLQPEDFATYYCQQSYSTPRTFCHGTKVTVL

SEQ ID NO: 523 - protein, 106 aa, synthetic construct
EIVLTQSPTTLSLSPGERATLSCRASQSVGRYLAWYQQRPGQAPRLLVFDTSNRAPGVPARFSGRGSGTLFTLTISSLEPEDSAVYFCQQRSSGLTFGGGTKVTVL

SEQ ID NO: 524 - protein, 106 aa, synthetic construct
EIVLTQSPTTLSLSPGERATLCCRASQSVGRYLAWYQQRPGQAPRLLVFDTSNRAPGVPARFSGRGSGTLFCLTISSLEPEDSAVYFCQQRSSGLTFGGGTKVTVL

SEQ ID NO: 525 - protein, 106 aa, synthetic construct
EIVLTQSPTTLSLSPGERATLSCCASQSVGRYLAWYQQRPGQAPRLLVFDTSNRAPGVPARFSGRGSGTCFTLTISSLEPEDSAVYFCQQRSSGLTFGGGTKVTVL

SEQ ID NO: 526 - protein, 106 aa, synthetic construct
EIVLTQSPTTLSLSPGERATLSCRASQSVGRYLAWYQQCPGQAPRLLVFDTSNRAPGVPARFSGRGSGTLFTLTISSLEPEDSCVYFCQQRSSGLTFGGGTKVTVL

SEQ ID NO: 527 - protein, 106 aa, synthetic construct
EIVCTQSPTTLSLSPGERATLSCRCSQSVGRYLAWYQQRPGQAPRLLVFDTSNRAPGVPARFSGRGSGTLFTLTISSLEPEDSAVYFCQQRSSGLTFGGGTKVTVL

SEQ ID NO: 528 - protein, 106 aa, synthetic construct
EIVCTQSPTTLSLSPGERATLSCRASQSVGRYLAWYQQRPGQAPRLLVFDTSNRAPGVPARFSGRGSGTLFTLTISSLEPEDSAVYFCQQRSSGLTCGGGTKVTVL

SEQ ID NO: 529 - protein, 106 aa, synthetic construct
EIVLCQSPTTLSLSPGERATLSCRASQSVGRYLAWYQQRPGQAPRLLVFDTSNRAPGVPARFSGRGSGTLFTLTISSLEPEDSAVYFCQQRSSGLTFGCGTKVTVL

SEQ ID NO: 530 - protein, 106 aa, synthetic construct
EIVLTCSPTTLSLSPGERATLSCRASQSVGRYLAWYQQRPGQAPRLLVFDTSNRAPGVPARFSGRGSGTLFTLTISSLEPEDSAVYFCQQRSSGLTFCGGTKVTVL

SEQ ID NO: 531 - protein, 107 aa, synthetic construct
DIQMTQSPPSLSAFVGDRVTITCRASESVGNSLSWYQLKPGKNPRLLVSGGSFLQSGVSARFSGSGAGTLFTLTITGLRLDDSATYYCQQSDAVPRTFGHGTKVSVL

SEQ ID NO: 532 - protein, 107 aa, synthetic construct
DIQMTQSPPSLSAFVGDRVTICCRASESVGNSLSWYQLKPGKNPRLLVSGGSFLQSGVSARFSGSGAGTLFCLTITGLRLDDSATYYCQQSDAVPRTFGHGTKVSVL

SEQ ID NO: 533 - protein, 107 aa, synthetic construct
DIQMTQSPPSLSAFVGDRVTITCCASESVGNSLSWYQLKPGKNPRLLVSGGSFLQSGVSARFSGSGAGTCFTLTITGLRLDDSATYYCQQSDAVPRTFGHGTKVSVL

SEQ ID NO: 534 - protein, 107 aa, synthetic construct
DIQMTQSPPSLSAFVGDRVTITCRASESVGNSLSWYQLCPGKNPRLLVSGGSFLQSGVSARFSGSGAGTLFTLTITGLRLDDSCTYYCQQSDAVPRTFGHGTKVSVL

SEQ ID NO: 535 - protein, 107 aa, synthetic construct
DIQCTQSPPSLSAFVGDRVTITCRCSESVGNSLSWYQLKPGKNPRLLVSGGSFLQSGVSARFSGSGAGTLFTLTITGLRLDDSATYYCQQSDAVPRTFGHGTKVSVL

SEQ ID NO: 536 - protein, 107 aa, synthetic construct
DIQCTQSPPSLSAFVGDRVTITCRASESVGNSLSWYQLKPGKNPRLLVSGGSFLQSGVSARFSGSGAGTLFTLTITGLRLDDSATYYCQQSDAVPRTCGHGTKVSVL

SEQ ID NO: 537 - protein, 107 aa, synthetic construct
DIQMCQSPPSLSAFVGDRVTITCRASESVGNSLSWYQLKPGKNPRLLVSGGSFLQSGVSARFSGSGAGTLFTLTITGLRLDDSATYYCQQSDAVPRTFGCGTKVSVL

SEQ ID NO: 538 - protein, 107 aa, synthetic construct
DIQMTCSPPSLSAFVGDRVTITCRASESVGNSLSWYQLKPGKNPRLLVSGGSFLQSGVSARFSGSGAGTLFTLTITGLRLDDSATYYCQQSDAVPRTFCHGTKVSVL

SEQ ID NO: 539 - protein, 108 aa, synthetic construct
EIVMTQSPATLSLSPGERATLSCRASQSVSSSSLAWYQQKPGQAPRLLIYGTSNRATGIPDRFSGSGSGTHFTLTINRLEPGDFAVYYCQQYGSSPRTFGQGTKVEIK

SEQ ID NO: 540 - protein, 108 aa, synthetic construct
EIVMTQSPATLSLSPGERATLCCRASQSVSSSSLAWYQQKPGQAPRLLIYGTSNRATGIPDRFSGSGSGTHFCLTINRLEPGDFAVYYCQQYGSSPRTFGQGTKVEIK

SEQ ID NO: 541 - protein, 108 aa, synthetic construct
EIVMTQSPATLSLSPGERATLSCCASQSVSSSSLAWYQQKPGQAPRLLIYGTSNRATGIPDRFSGSGSGTCFTLTINRLEPGDFAVYYCQQYGSSPRTFGQGTKVEIK

SEQ ID NO: 542 - protein, 108 aa, synthetic construct
EIVMTQSPATLSLSPGERATLSCRASQSVSSSSLAWYQQCPGQAPRLLIYGTSNRATGIPDRFSGSGSGTHFTLTINRLEPGDFCVYYCQQYGSSPRTFGQGTKVEIK

SEQ ID NO: 543 - protein, 108 aa, synthetic construct
EIVCTQSPATLSLSPGERATLSCRCSQSVSSSSLAWYQQKPGQAPRLLIYGTSNRATGIPDRFSGSGSGTHFTLTINRLEPGDFAVYYCQQYGSSPRTFGQGTKVEIK

SEQ ID NO: 544 - protein, 108 aa, synthetic construct
EIVCTQSPATLSLSPGERATLSCRASQSVSSSSLAWYQQKPGQAPRLLIYGTSNRATGIPDRFSGSGSGTHFTLTINRLEPGDFAVYYCQQYGSSPRTCGQGTKVEIK

SEQ ID NO: 545 - protein, 108 aa, synthetic construct
EIVMCQSPATLSLSPGERATLSCRASQSVSSSSLAWYQQKPGQAPRLLIYGTSNRATGIPDRFSGSGSGTHFTLTINRLEPGDFAVYYCQQYGSSPRTFGCGTKVEIK

SEQ ID NO: 546 - protein, 108 aa, synthetic construct
EIVMTCSPATLSLSPGERATLSCRASQSVSSSSLAWYQQKPGQAPRLLIYGTSNRATGIPDRFSGSGSGTHFTLTINRLEPGDFAVYYCQQYGSSPRTFCQGTKVEIK

SEQ ID NO: 547 - protein, 107 aa, synthetic construct
DIQMTQSPSSLSASVGDRVTITCRASQDIRTDLDWFQQRPGRAPHRLIYGASSLQGGVPSRFSGSGSGTEFTLTISGLQPEDFATYYCLQHHTYPRTFGLGTKVTVL

SEQ ID NO: 548 - protein, 107 aa, synthetic construct
DIQMTQSPSSLSASVGDRVTICCRASQDIRTDLDWFQQRPGRAPHRLIYGASSLQGGVPSRFSGSGSGTEFCLTISGLQPEDFATYYCLQHHTYPRTFGLGTKVTVL

SEQ ID NO: 549 - protein, 107 aa, synthetic construct
DIQMTQSPSSLSASVGDRVTITCCASQDIRTDLDWFQQRPGRAPHRLIYGASSLQGGVPSRFSGSGSGTCFTLTISGLQPEDFATYYCLQHHTYPRTFGLGTKVTVL

SEQ ID NO: 550 - protein, 107 aa, synthetic construct
DIQMTQSPSSLSASVGDRVTITCRASQDIRTDLDWFQQCPGRAPHRLIYGASSLQGGVPSRFSGSGSGTEFTLTISGLQPEDFCTYYCLQHHTYPRTFGLGTKVTVL

SEQ ID NO: 551 - protein, 107 aa, synthetic construct
DIQCTQSPSSLSASVGDRVTITCRCSQDIRTDLDWFQQRPGRAPHRLIYGASSLQGGVPSRFSGSGSGTEFTLTISGLQPEDFATYYCLQHHTYPRTFGLGTKVTVL

SEQ ID NO: 552 - protein, 107 aa, synthetic construct
DIQCTQSPSSLSASVGDRVTITCRASQDIRTDLDWFQQRPGRAPHRLIYGASSLQGGVPSRFSGSGSGTEFTLTISGLQPEDFATYYCLQHHTYPRTCGLGTKVTVL

SEQ ID NO: 553 - protein, 107 aa, synthetic construct
DIQMCQSPSSLSASVGDRVTITCRASQDIRTDLDWFQQRPGRAPHRLIYGASSLQGGVPSRFSGSGSGTEFTLTISGLQPEDFATYYCLQHHTYPRTFGCGTKVTVL

SEQ ID NO: 554 - protein, 107 aa, synthetic construct
DIQMTCSPSSLSASVGDRVTITCRASQDIRTDLDWFQQRPGRAPHRLIYGASSLQGGVPSRFSGSGSGTEFTLTISGLQPEDFATYYCLQHHTYPRTFCLGTKVTVL

SEQ ID NO: 555 - protein, 107 aa, synthetic construct
EIVMTQSPVTLSLSPGERATLSCRASQSVGTSLAWYQQKPGQAPRLLIYDASNRATGISARFSGSGSGTDFTLTISSLEPEDFAVYYCQQRYNWPRTFGGGTKVTVL

SEQ ID NO: 556 - protein, 107 aa, synthetic construct
EIVMTQSPVTLSLSPGERATLCCRASQSVGTSLAWYQQKPGQAPRLLIYDASNRATGISARFSGSGSGTDFCLTISSLEPEDFAVYYCQQRYNWPRTFGGGTKVTVL

SEQ ID NO: 557 - protein, 107 aa, synthetic construct
EIVMTQSPVTLSLSPGERATLSCCASQSVGTSLAWYQQKPGQAPRLLIYDASNRATGISARFSGSGSGTCFTLTISSLEPEDFAVYYCQQRYNWPRTFGGGTKVTVL

SEQ ID NO: 558 - protein, 107 aa, synthetic construct
EIVMTQSPVTLSLSPGERATLSCRASQSVGTSLAWYQQCPGQAPRLLIYDASNRATGISARFSGSGSGTDFTLTISSLEPEDFCVYYCQQRYNWPRTFGGGTKVTVL

SEQ ID NO: 559 - protein, 107 aa, synthetic construct
EIVCTQSPVTLSLSPGERATLSCRCSQSVGTSLAWYQQKPGQAPRLLIYDASNRATGISARFSGSGSGTDFTLTISSLEPEDFAVYYCQQRYNWPRTFGGGTKVTVL

SEQ ID NO: 560 - protein, 108 aa, synthetic construct
EIVCMTQSPVTLSLSPGERATLSCRASQSVGTSLAWYQQKPGQAPRLLIYDASNRATGISARFSGSGSGTDFTLTISSLEPEDFAVYYCQQRYNWPRTCGGGTKVTVL

SEQ ID NO: 561 - protein, 107 aa, synthetic construct
EIVMCQSPVTLSLSPGERATLSCRASQSVGTSLAWYQQKPGQAPRLLIYDASNRATGISARFSGSGSGTDFTLTISSLEPEDFAVYYCQQRYNWPRTFGCGTKVTVL

SEQ ID NO: 562 - protein, 107 aa, synthetic construct
EIVMTCSPVTLSLSPGERATLSCRASQSVGTSLAWYQQKPGQAPRLLIYDASNRATGISARFSGSGSGTDFTLTISSLEPEDFAVYYCQQRYNWPRTFCGGTKVTVL

SEQ ID NO: 563 - protein, 107 aa, synthetic construct
ETTLTQSPATLSVSPGERATFSCRASQSVSNNLAWYQQKPGQAPRLLIYGASSRTTGIPDRFSASGSGTDFTLTISRLEPEDFAVYYCQQYDTSPRTFGQGTKVEIK

SEQ ID NO: 564 - protein, 107 aa, synthetic construct
ETTLTQSPATLSVSPGERATFCCRASQSVSNNLAWYQQKPGQAPRLLIYGASSRTTGIPDRFSASGSGTDFCLTISRLEPEDFAVYYCQQYDTSPRTFGQGTKVEIK

SEQ ID NO: 565 - protein, 107 aa, synthetic construct
ETTLTQSPATLSVSPGERATFSCCASQSVSNNLAWYQQKPGQAPRLLIYGASSRTTGIPDRFSASGSGTCFTLTISRLEPEDFAVYYCQQYDTSPRTFGQGTKVEIK

SEQ ID NO: 566 - protein, 107 aa, synthetic construct
ETTLTQSPATLSVSPGERATFSCRASQSVSNNLAWYQQCPGQAPRLLIYGASSRTTGIPDRFSASGSGTDFTLTISRLEPEDFCVYYCQQYDTSPRTFGQGTKVEIK

SEQ ID NO: 567 - protein, 107 aa, synthetic construct
ETTCTQSPATLSVSPGERATFSCRCSQSVSNNLAWYQQKPGQAPRLLIYGASSRTTGIPDRFSASGSGTDFTLTISRLEPEDFAVYYCQQYDTSPRTFGQGTKVEIK

SEQ ID NO: 568 - protein, 107 aa, synthetic construct
ETTCTQSPATLSVSPGERATFSCRASQSVSNNLAWYQQKPGQAPRLLIYGASSRTTGIPDRFSASGSGTDFTLTISRLEPEDFAVYYCQQYDTSPRTCGQGTKVEIK

SEQ ID NO: 569 - protein, 107 aa, synthetic construct
ETTLCQSPATLSVSPGERATFSCRASQSVSNNLAWYQQKPGQAPRLLIYGASSRTTGIPDRFSASGSGTDFTLTISRLEPEDFAVYYCQQYDTSPRTFGCGTKVEIK

SEQ ID NO: 570 - protein, 107 aa, synthetic construct
ETTLTCSPATLSVSPGERATFSCRASQSVSNNLAWYQQKPGQAPRLLIYGASSRTTGIPDRFSASGSGTDFTLTISRLEPEDFAVYYCQQYDTSPRTFCQGTKVEIK

SEQ ID NO: 571 - protein, 107 aa, synthetic construct
EIVMTQSPGTLSLSPGDRATLSCRASQDISTYLAWYQQKPGQAPRLLIYSASRRATGIPDRFSGSGSGTDFTLTISRLEPEDFAVYYCQQYSSSPKTFGQGTKVTVL

SEQ ID NO: 572 - protein, 107 aa, synthetic construct
EIVMTQSPGTLSLSPGDRATLCCRASQDISTYLAWYQQKPGQAPRLLIYSASRRATGIPDRFSGSGSGTDFCLTISRLEPEDFAVYYCQQYSSSPKTFGQGTKVTVL

SEQ ID NO: 573 - protein, 107 aa, synthetic construct
EIVMTQSPGTLSLSPGDRATLSCCASQDISTYLAWYQQKPGQAPRLLIYSASRRATGIPDRFSGSGSGTCFTLTISRLEPEDFAVYYCQQYSSSPKTFGQGTKVTVL

SEQ ID NO: 574 - protein, 107 aa, synthetic construct
EIVMTQSPGTLSLSPGDRATLSCRASQDISTYLAWYQQCPGQAPRLLIYSASRRATGIPDRFSGSGSGTDFTLTISRLEPEDFCVYYCQQYSSSPKTFGQGTKVTVL

SEQ ID NO: 575 - protein, 107 aa, synthetic construct
EIVCTQSPGTLSLSPGDRATLSCRCSQDISTYLAWYQQKPGQAPRLLIYSASRRATGIPDRFSGSGSGTDFTLTISRLEPEDFAVYYCQQYSSSPKTFGQGTKVTVL

SEQ ID NO: 576 - protein, 107 aa, synthetic construct
EIVCTQSPGTLSLSPGDRATLSCRASQDISTYLAWYQQKPGQAPRLLIYSASRRATGIPDRFSGSGSGTDFTLTISRLEPEDFAVYYCQQYSSSPKTCGQGTKVTVL

SEQ ID NO: 577 - protein, 107 aa, synthetic construct
EIVMCQSPGTLSLSPGDRATLSCRASQDISTYLAWYQQKPGQAPRLLIYSASRRATGIPDRFSGSGSGTDFTLTISRLEPEDFAVYYCQQYSSSPKTFGCGTKVTVL

SEQ ID NO: 578 - protein, 107 aa, synthetic construct
EIVMTCSPGTLSLSPGDRATLSCRASQDISTYLAWYQQKPGQAPRLLIYSASRRATGIPDRFSGSGSGTDFTLTISRLEPEDFAVYYCQQYSSSPKTFCQGTKVTVL

SEQ ID NO: 579 - protein, 107 aa, synthetic construct
ETTLTQSPGTLSLSPGERATLSCRASQSVSKNLAWYQKKPGQSPRLLIHSISTRATGVPARFSGGGSGTDFTLTISRLEPEDFAVYYCQQYGNSPQTFGQGTKVEIK

SEQ ID NO: 580 - protein, 107 aa, synthetic construct
ETTLTQSPGTLSLSPGERATLCCRASQSVSKNLAWYQKKPGQSPRLLIHSISTRATGVPARFSGGGSGTDFCLTISRLEPEDFAVYYCQQYGNSPQTFGQGTKVEIK

SEQ ID NO: 581 - protein, 107 aa, synthetic construct
ETTLTQSPGTLSLSPGERATLSCCASQSVSKNLAWYQKKPGQSPRLLIHSISTRATGVPARFSGGGSGTCFTLTISRLEPEDFAVYYCQQYGNSPQTFGQGTKVEIK

SEQ ID NO: 582 - protein, 107 aa, synthetic construct
ETTLTQSPGTLSLSPGERATLSCRASQSVSKNLAWYQKKCGQSPRLLIHSISTRATGVPARFSGGGSGTDFTLTISRLEPEDFCVYYCQQYGNSPQTFGQGTKVEIK

SEQ ID NO: 583 - protein, 107 aa, synthetic construct
ETTCTQSPGTLSLSPGERATLSCRCSQSVSKNLAWYQKKPGQSPRLLIHSISTRATGVPARFSGGGSGTDFTLTISRLEPEDFAVYYCQQYGNSPQTFGQGTKVEIK

SEQ ID NO: 584 - protein, 107 aa, synthetic construct
ETTCTQSPGTLSLSPGERATLSCRASQSVSKNLAWYQKKPGQSPRLLIHSISTRATGVPARFSGGGSGTDFTLTISRLEPEDFAVYYCQQYGNSPQTCGQGTKVEIK

SEQ ID NO: 585 - protein, 107 aa, synthetic construct
ETTLCQSPGTLSLSPGERATLSCRASQSVSKNLAWYQKKPGQSPRLLIHSISTRATGVPARFSGGGSGTDFTLTISRLEPEDFAVYYCQQYGNSPQTFGCGTKVEIK

SEQ ID NO: 586 - protein, 107 aa, synthetic construct
ETTLTCSPGTLSLSPGERATLSCRASQSVSKNLAWYQKKPGQSPRLLIHSISTRATGVPARFSGGGSGTDFTLTISRLEPEDFAVYYCQQYGNSPQTFCQGTKVEIK

SEQ ID NO: 587 - protein, 110 aa, synthetic construct
QSVVTQPPSVSAAPGQRVTISCSGSSYNIGENSVSWYQQLPGTAPKLLIYGNDKRPSGIPDRFSGSKSGTSATLGITGLQTGDEADYYCGTWDSNLRASVFGGGTKVTVL

SEQ ID NO: 588 - protein, 110 aa, synthetic construct
QSVVTQPPSVSAAPGQRVTICCSGSSYNIGENSVSWYQQLPGTAPKLLIYGNDKRPSGIPDRFSGSKSGTSACLGITGLQTGDEADYYCGTWDSNLRASVFGGGTKVTVL

SEQ ID NO: 589 - protein, 110 aa, synthetic construct
QSVVTQPPSVSAAPGQRVTISCCGSSYNIGENSVSWYQQLPGTAPKLLIYGNDKRPSGIPDRFSGSKSGTCATLGITGLQTGDEADYYCGTWDSNLRASVFGGGTKVTVL

SEQ ID NO: 590 - protein, 110 aa, synthetic construct
QSVVTQPPSVSAAPGQRVTISCSGSSYNIGENSVSWYQQCPGTAPKLLIYGNDKRPSGIPDRFSGSKSGTSATLGITGLQTGDECDYYCGTWDSNLRASVFGGGTKVTVL

SEQ ID NO: 591 - protein, 110 aa, synthetic construct
QSVCTQPPSVSAAPGQRVTISCSCSSYNIGENSVSWYQQLPGTAPKLLIYGNDKRPSGIPDRFSGSKSGTSATLGITGLQTGDEADYYCGTWDSNLRASVFGGGTKVTVL

SEQ ID NO: 592 - protein, 110 aa, synthetic construct
QSVCTQPPSVSAAPGQRVTISCSGSSYNIGENSVSWYQQLPGTAPKLLIYGNDKRPSGIPDRFSGSKSGTSATLGITGLQTGDEADYYCGTWDSNLRASVCGGGTKVTVL

SEQ ID NO: 593 - protein, 110 aa, synthetic construct
QSVVCQPPSVSAAPGQRVTISCSGSSYNIGENSVSWYQQLPGTAPKLLIYGNDKRPSGIPDRFSGSKSGTSATLGITGLQTGDEADYYCGTWDSNLRASVFGCGTKVTVL

SEQ ID NO: 594 - protein, 110 aa, synthetic construct
QSVVTCPPSVSAAPGQRVTISCSGSSYNIGENSVSWYQQLPGTAPKLLIYGNDKRPSGIPDRFSGSKSGTSATLGITGLQTGDEADYYCGTWDSNLRASVFCGGTKVTVL

SEQ ID NO: 595 - protein, 107 aa, synthetic construct
ETTLTQSPGTLSLSPGERATLSCRASQSVRNNLAWYQQRPGQAPRLLIYGASTRATGIPARFSGSGSGTDFTLTISSLQVEDVAVYYCQQYYTTPKTFGQGTKVEIK

SEQ ID NO: 596 - protein, 107 aa, synthetic construct
ETTLTQSPGTLSLSPGERATLCCRASQSVRNNLAWYQQRPGQAPRLLIYGASTRATGIPARFSGSGSGTDFCLTISSLQVEDVAVYYCQQYYTTPKTFGQGTKVEIK

SEQ ID NO: 597 - protein, 107 aa, synthetic construct
ETTLTQSPGTLSLSPGERATLSCCASQSVRNNLAWYQQRPGQAPRLLIYGASTRATGIPARFSGSGSGTCFTLTISSLQVEDVAVYYCQQYYTTPKTFGQGTKVEIK

SEQ ID NO: 598 - protein, 107 aa, synthetic construct
ETTLTQSPGTLSLSPGERATLSCRASQSVRNNLAWYQQCPGQAPRLLIYGASTRATGIPARFSGSGSGTDFTLTISSLQVEDVCVYYCQQYYTTPKTFGQGTKVEIK

SEQ ID NO: 599 - protein, 107 aa, synthetic construct
ETTCTQSPGTLSLSPGERATLSCRCSQSVRNNLAWYQQRPGQAPRLLIYGASTRATGIPARFSGSGSGTDFTLTISSLQVEDVAVYYCQQYYTTPKTFGQGTKVEIK

SEQ ID NO: 600 - protein, 107 aa, synthetic construct
ETTCTQSPGTLSLSPGERATLSCRASQSVRNNLAWYQQRPGQAPRLLIYGASTRATGIPARFSGSGSGTDFTLTISSLQVEDVAVYYCQQYYTTPKTCGQGTKVEIK

SEQ ID NO: 601 - protein, 107 aa, synthetic construct
ETTLCQSPGTLSLSPGERATLSCRASQSVRNNLAWYQQRPGQAPRLLIYGASTRATGIPARFSGSGSGTDFTLTISSLQVEDVAVYYCQQYYTTPKTFGCGTKVEIK

SEQ ID NO: 602 - protein, 107 aa, synthetic construct
ETTLTCSPGTLSLSPGERATLSCRASQSVRNNLAWYQQRPGQAPRLLIYGASTRATGIPARFSGSGSGTDFTLTISSLQVEDVAVYYCQQYYTTPKTFCQGTKVEIK

SEQ ID NO: 603 - DNA, 33 bp, synthetic construct
  source 1..33: mol_type = other DNA
accgctataaataaagcagacccactccagtcc

SEQ ID NO: 604 - DNA, 31 bp, synthetic construct
  source 1..31: mol_type = other DNA
ggaattgtctctggagcaggtgaatcggccc

SEQ ID NO: 605 - DNA, 34 bp, synthetic construct
  source 1..34: mol_type = other DNA
agcctggcggacccagcacatgtagtagtcactg

SEQ ID NO: 606 - DNA, 33 bp, synthetic construct
  source 1..33: mol_type = other DNA
accaccgctataaatgcatgagacccactccag

SEQ ID NO: 607 - DNA, 34 bp, synthetic construct
  source 1..34: mol_type = other DNA
gttcatttgaagatagcacgtgttcttggaattg

SEQ ID NO: 608 - DNA, 32 bp, synthetic construct
  source 1..32: mol_type = other DNA
catgcagtgcacaggaagtgcagctggtgcag

SEQ ID NO: 609 - DNA, 49 bp, synthetic construct
  source 1..49: mol_type = other DNA
catgcagcggccgctttggaaccagagcctgaagagacggtgaccattg

SEQ ID NO: 610 - DNA, 29 bp, synthetic construct
  source 1..29: mol_type = other DNA
catgcagtgcacaggtgcagctgcaggag

SEQ ID NO: 611 - DNA, 48 bp, synthetic construct
  source 1..48: mol_type = other DNA
catgcagcggccgctttggaaccagagcctgaggagacggtgaccgtg

SEQ ID NO: 612 - DNA, 29 bp, synthetic construct
  source 1..29: mol_type = other DNA
catgcagtgcacaggtccagctggtgcag

SEQ ID NO: 613 - DNA, 49 bp, synthetic construct; identical to SEQ ID NO: 609
  source 1..49: mol_type = other DNA

SEQ ID NO: 614 - DNA, 27 bp, synthetic construct
  source 1..27: mol_type = other DNA
gtgaaaaaattattattcgcaattcct

SEQ ID NO: 615 - DNA, 20 bp, synthetic construct
  source 1..20: mol_type = other DNA
ccctcatagttagcgtaacg